(19) United States
(12) Patent Application Publication
(10) Pub. No.: US 2012/0083473 A1
(43) Pub. Date: Apr. 5, 2012

(54) TREATMENT OF CONDITIONS BY TOLL-LIKE RECEPTOR MODULATORS

(76) Inventors: Johanna Holldack, Melide (CH); Alberto Mantovani, Milano (IT); Antonio Sica, Rho (IT); Nadia Passini, Lavena Ponte Tresa (IT); Roberto Maj, Saronno (IT); Alcide Barberis, Breganzona (CH)

(21) Appl. No.: 13/237,744

(22) Filed: Sep. 20, 2011

Related U.S. Application Data
(60) Provisional application No. 61/385,105, filed on Sep. 21, 2010.

(51) Int. Cl. A61K 31/52; A61P 43/00; A61P 29/00; A61P 19/02; A61P 11/00; A61P 13/12; A61P 25/00; A61K 31/655; A61P 37/00

(52) U.S. Cl. 514/150; 514/263.2; 514/263.37; 514/263.21; 514/263.31

(57) ABSTRACT

Provided herein are methods for treating certain conditions, including fibrosis, inflammatory, and autoimmune conditions, with conjugated compounds having Toll-like receptor modulatory activity.

### RELATED PATENT APPLICATIONS

Priority is claimed to U.S. Provisional Patent Application Ser. No. 61/385,105, filed Sep. 21, 2010, and entitled “Treatment of Conditions by Toll-like Receptor Modulators” which is referred to and incorporated by reference herein in its entirety.

### FIELD

The technology in part relates to methods for using molecules that modulate the function of Toll-Like Receptor 7 (TLR7) and methods for treating diseases by administering such molecules to subjects in need thereof.

### SUMMARY

Provided herein are methods for treating a condition in a subject, which comprise administering a compound having a structure according to Formula I, II or III to a subject in need thereof in an amount effective to prevent, ameliorate, inhibit or treat the condition, where the condition may be prevented, ameliorated, inhibited or treated by inducing a relatively lower M1 cytokine level and relatively higher M2 cytokine level state in the subject. In certain embodiments, the condition may be prevented, ameliorated, inhibited or treated by inducing macrophage M1 to M2 skewing, that is shifting the activity from pro-inflammatory cells of an immune response to cells involved in tissue repair and remodeling.

Examples of conditions that may be treated by administering a compound described herein to a subject, include, but are not limited to, a fibrosis condition. Fibrosis conditions are characterized by the formation of excessive connective or fibrotic tissue in an organ or tissue. Fibrosis conditions may have the same, or different, causes, and may be the result of another condition. Examples of fibrosis conditions include Crohn's disease, cirrhosis, renal fibrosis, endomyocardial fibrosis, keloid, mediastinal fibrosis, myelofibrosis, myocardial infarction, nephrogenic systemic fibrosis, progressive massive fibrosis, pulmonary and idiopathic pulmonary fibrosis, retroperitoneal fibrosis, lung fibrosis, cystic fibrosis, sarcoidosis, scleroderma/systemic sclerosis, and multiple sclerosis. In certain examples, fibrosis conditions include cirrhosis, renal fibrosis, endomyocardial fibrosis, keloid, mediastinal fibrosis, myelofibrosis, myocardial infarction, nephrogenic systemic fibrosis, progressive massive fibrosis, pulmonary and idiopathic pulmonary fibrosis, retroperitoneal fibrosis, lung fibrosis, sarcoidosis, and scleroderma/systemic sclerosis. In some examples, fibrosis conditions include cirrhosis, renal fibrosis, keloid, mediastinal fibrosis, myelofibrosis, nephrogenic systemic fibrosis, retroperitoneal fibrosis, sarcoidosis, and scleroderma/systemic sclerosis. In certain examples, fibrosis conditions include endomyocardial fibrosis, myocardial infarction, progressive massive fibrosis, pulmonary and idiopathic pulmonary fibrosis, and lung fibrosis. In certain examples, the fibrosis condition is lung fibrosis.

Some examples of conditions that may be treated by administering a compound described herein to a subject, include, but are not limited to an organ failure condition. Examples of organ failure conditions include, for example, failure in any organ, including, for example, a liver failure condition, a kidney failure condition, and a lung failure condition. In certain examples, the condition is kidney failure (e.g., characterized by a variation in a level of one or more of creatinine, blood urea-nitrogen, red blood cells, white blood cells, leukocytes, protein, microalbumin, parathyroid hormone, and/or cystatin C).

Some examples of conditions that may be treated by administering a compound described herein to a subject include, but are not limited to, a condition requiring tissue repair, a tissue remodeling and/or wound healing condition, ulcerative colitis; a liver failure condition (e.g., characterized by a variation in a level of one or more liver blood enzymes such as amniotransferases for example asparatate and alaninie transferases, alkaline phoshatase, 5′-nucleotidase, and gamma glutamyltranspeptidase).

Certain examples of conditions that may be treated by administering a compound described herein to a subject include, but are not limited to, an inflammatory or autoimmune condition. These conditions include various types of specific conditions, which may, or may not, have the same cause or symptoms. Examples of inflammatory or autoimmune conditions include, but are not limited to, asthma, reactive airway disease, skin inflammation, rheumatoid arthritis, a kidney failure condition (e.g., characterized by a variation in a level of one or more of creatinine, blood urea-nitrogen, red blood cells, white blood cells, leukocytes, protein, microalbumin, parathyroid hormone, and/or cystatin C); a lung damage condition (e.g., characterized by a variation in a level of one or more of lactate dehydrogenase, isoenzymes, glucose-6-phosphate-dehydrorgenase, lysosomal acid hydrolases, alkaline phosphatase, glutathione peroxidase/reductase, angiotension converting enzyme, sialic acid and phagocytic cells); an atherosclerosis or vascular condition (e.g., characterized by a variation in a level of one or more of lipoproteins, apolipoproteins, and/or glycosaminoglycan); an inflammatory bowel condition (e.g., characterized by a variation in a level of one or more of anti-neutrophil cytoplasmic autoantibodies, anti-Saccharomyces cerevisiae antibodies, Escherichia coli-related OmpC, pserdomonas fluorescens and flagellin CBir1); a post surgical adhesion condition; a peritoneal adhesion condition (e.g., characterized by a variation in a level of one or more of inflammatory cytokines, and optionally induced by surgery, chemical peritonitis, radiotherapy, and/or foreign body reaction); and a tissue repair and or remodeling condition. In certain examples, the inflammatory or autoimmune condition includes rheumatoid arthritis, a kidney failure condition (e.g., characterized by a variation in a level of one or more of creatinine, blood urea-nitrogen, red blood cells, white blood cells, leukocytes, protein, microalbumin, parathyroid hormone, and/or cystatin C); a lung damage condition (e.g., characterized by a variation in a level of one or more of lactate dehydrogenase, isoenzymes, glucose-6-phosphate-dehydrorgenase, lysosomal acid hydrolases, alkaline phosphatase, glutathione peroxidase/reductase, angiotension converting enzyme, sialic acid and phagocytic cells); an atherosclerosis or vascular condition (e.g., characterized by a variation in a level of one or more of lipoproteins, apolipoproteins, and/or glycosaminoglycan); an inflammatory bowel condition (e.g., characterized by a variation in a level of one or more of anti-neutrophil cytoplasmic autoantibodies, anti-Saccharomyces cerevisiae antibodies, Escherichia coli-related OmpC, pserdomonas fluorescens and flagellin CBir1); a post surgical adhesion condition; a peritoneal adhesion condition (e.g., characterized by a variation in a level of one or more of inflammatory cytokines, and optionally induced by surgery, chemical peritonitis, radiotherapy, and/or foreign body reaction); and a tissue repair and or remodeling condition. In certain examples, the condition is rheumatoid arthritis.

Provided herein are small molecule conjugates that can modulate an activity of one or more toll-like receptors (e.g., the conjugates are agonists, antagonists, or both). The small molecule conjugates described herein are also considered to be partial agonists. Partial agonists bind and activate a given receptor, but have only partial efficacy relative to a full agonist. The small molecule conjugates described herein may also be considered ligands, which display both agonistic and antagonistic effects—when both a full agonist and partial agonist are present, the partial agonist actually acts as a competitive antagonist, competing with the full agonist for receptor occupancy and producing a net decrease (relative inhibition) in the receptor activation observed with the full agonist alone. Clinically, partial agonists can activate receptors to give a desired submaximal response when inadequate amounts of the endogenous ligand are present, or they can reduce the overstimulation of receptors when excess amounts of the endogenous ligand are present. The term “toll-like receptor” (TLR) refers to a member of a family of receptors that bind to pathogen-associated molecular patterns (PAMPs) and facilitate an immune response in a mammal. Ten mammalian TLRs are known, e.g., TLR1-10. The term “toll-like receptor agonist” (TLR agonist) refers to a molecule that interacts with a TLR and stimulates the activity of the receptor. Synthetic TLR agonists are chemical compounds that are designed to interact with a TLR and stimulate the activity of the receptor. Examples of TLR agonists include a TLR-7 agonist, TLR-3 agonist or TLR-9 agonist. The term “toll-like receptor antagonist” (TLR antagonist) refers to a molecule that interacts with a TLR and inhibits or neutralizes the signaling activity of the receptor. Synthetic TLR antagonists are chemical compounds designed to interact with a TLR and interfere with the activity of the receptor. Examples of TLR antagonists include a TLR-7 antagonist, TLR-3 antagonist or TLR-9 antagonist.

Thus, in an embodiment, provided herein are methods for treating a condition in a subject, comprising administering to the subject a compound having a structure according to Formula I:

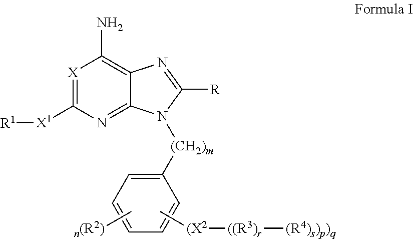

or a pharmaceutically acceptable salt thereof, including a hydrate thereof, wherein:
X is N or CR²;
R is —OR¹, —SR¹, or —NR^{a}R^{b},
X¹ is a bond or is —O—, —S—, or —NR^{c}—;
R^{c} is hydrogen, C1-C10 alkyl or substituted C1-C10 alkyl, or R^{c} and R¹ taken together with the nitrogen atom can form a heterocyclic ring or a substituted heterocyclic ring;
R¹ is hydrogen, C1-C10 alkyl, substituted C1-C10 alkyl, C1-C10 alkoxy, substituted C1-C10 alkoxy, C1-C10 alkyl C1-C10 alkoxy, substituted C1-C10 alkyl C1-C10 alkoxy, C5-C10 aryl, substituted C5-C10 aryl, C5-C9 heterocyclic, substituted C5-C9 heterocyclic, C3-C9 carbocyclic or substituted C3-C9 carbocyclic;
each R² independently is hydrogen, —OH, C1-C6 alkyl, substituted C1-C6 alkyl, C1-C6 alkoxy, substituted C1-C6 alkoxy, —C(O)—C1-C6 alkyl (alkanoyl), substituted —C(O)—C1-C6 alkyl, —C(O)—C6-C10 aryl (aroyl), substituted —C(O)—C6-C10 aryl, —C(O)OH (carboxyl), —C(O)O—C1-C6 alkyl (alkoxycarbonyl), substituted —C(O)O—C1-C6 alkyl, —NR^{a}R^{b}, —C(O)NR^{a}R^{b} (carbamoyl), substituted C(O)NR^{a}R^{b}, halo, nitro, or cyano;
the substituents on the alkyl, aryl or heterocyclic groups are hydroxy, C1-C6 alkyl, hydroxy C1-C6 alkylene, C1-C6 alkoxy, C3-C6 cycloalkyl, C1-C6 alkoxy C1-C6 alkylene, amino, cyano, halogen, or aryl;

each R^{a} and R^{b} is independently hydrogen, C1-C6 alkyl, C3-C8 cycloalkyl, C1-C6 alkoxy, halo C1-C6 alkyl, C3-C8 cycloalkyl C1-C6 alkyl, C1-C6 alkanoyl, hydroxy C1-C6 alkyl, aryl, aryl C1-C6 alkyl, Het, Het C1-C6 alkyl, or C1-C6 alkoxycarbonyl;
each X² independently is a bond or a linking group;
each R³ independently is a polyethylene glycol (PEG) moiety;
each R⁴ independently is H, —C1-C6 alkyl, —C1-C6 alkoxy, —NR^{a}R^{b}, —OH, —CN, —COOH, —COOR¹, —C1-C6 alkyl-NR^{a}R^{b}, —C1-C6 alkyl-OH, —C1-C6 alkyl-CN, —C1-C6 alkyl-COOH, —C1-C6 alkyl-COOR¹, -optionally substituted 5-6 membered ring, or —C1-C6 alkyl-optionally substituted 5-6 membered ring;
m is 1, 2, 3, 4, 5, 6, 7, 8, 9 or 10;
n is 0, 1, 2, 3 or 4;
p is 1 to 100;
q is 1, 2, 3, 4 or 5
r is 1 to 1,000;
s is 1 to 1,000; and
the sum of n and q equals 5
   or a pharmaceutically acceptable salt, tautomer, or hydrate thereof.

In certain embodiments, a method is provided for treating a condition in a subject, comprising administering to the subject a compound having a structure according to Formula II:

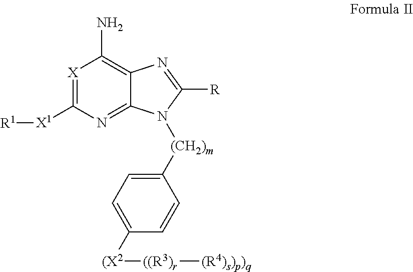

or a pharmaceutically acceptable salt thereof, or a hydrate thereof, where X, X¹, X², R, R¹, R², R³, R⁴, m, n, p, q, r and s embodiments are described above for Formula I.

In certain embodiments, a method is provided for treating a condition in a subject, comprising administering to the subject a compound having a structure according to Formula III:

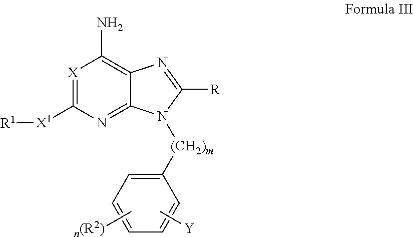

or a pharmaceutically acceptable salt thereof, or a hydrate thereof, where:
X, X¹, R, R¹ and R² embodiments are described above for Formula I;
Y is —X²—((R³)ᵣ—(X³)ₜ—(X⁴)ₛ)ₚ)_{q},
   —X²—((R³)ᵣ—(X³)ₜ'(X⁴)ₛ—(R⁴)ᵤ)ₚ)_{q},
   —X²—((X⁴)ₛ—(X³)ₜ—(R³)ᵣ)ₚ)_{q}, or
   —X²—((X⁴)ₛ—(X³)ₜ—(R³)ᵣ—(R⁴)ᵤ)ₚ)_{q},
R³, R⁴, m, n, p, q, r and s embodiments are described above for Formula I;
each X³ independently is a bond or linking group;
each X⁴ independently is a macromolecule;
t is 1 to 1,000; and
   u is 1 to 1,000.

In certain embodiments, the compound is administered to a human subject in need thereof in an amount effective to prevent, inhibit or treat the condition.

In some embodiments, X is N. In certain embodiments, X¹ is oxygen, and in some embodiments, R¹ is a substituted C1-C10 alkyl, such as a C1-C10 alkyl C1-C10 alkoxy moiety (e.g., —CH₂CH₂OCH₃). R¹ in some embodiments consists of six or fewer non-hydrogen atoms. In some embodiments, n is 4 and R² is hydrogen in each instance.

In certain embodiments, X² and/or X³ independently is an amido linking group (e.g., —C(O)NH— or —NH(O)C—); alkyl amido linking group (e.g., —C1-C6 alkyl-C(O)NH—, —C1-C6 alkyl-NH(O)C—, —C(O)NH—C1-C6 alkyl-, —NH(O)C—C1-C6 alkyl-, —C1-C6 alkyl-NH(O)C—C1-C6 alkyl-, or —C1-C6 alkyl-C(O)NH—C1-C6 alkyl-); or substituted 5-6 membered ring (e.g., aryl ring, heteroaryl ring (e.g., tetrazole, pyridyl, 2,5-pyrrolidinedione (e.g., 2,5-pyrrolidinedione substituted with a substituted phenyl moiety)), carbocyclic ring, or heterocyclic ring).

A PEG moiety can include one or more PEG units. A PEG moiety can include about 1 to about 1,000 PEG units, including, without limitation, about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 50, 60, 70, 80, 90, 100, 200, 300, 400, 500, 600, 700, 800 or 900 units, in some embodiments. A PEG unit is —O—CH₂—CH₂— or —CH₂—CH₂—O— in certain embodiments.

In some embodiments, p is about 1 to about 100, and sometimes can be, without limitation, about 2, 3, 4, 5, 6, 7, 8, 9, 10, 11, 12, 13, 14, 15, 16, 17, 18, 19, 20, 30, 40, 50, 60, 70, 80, 90, or 100. In some embodiments, r is about 5 to about 100, and sometimes r is about 5 to about 50 or about 5 to about 25. In certain embodiments, r is about 5 to about 15 and sometimes r is about 10. In some embodiments, r can sometimes be, without limitation, about 2, 3, 4, 5, 6, 7, 8, 9, 10, 11, 12, 13, 14, 15, 16, 17, 18, 19, 20, 30, 40, 50, 60, 70, 80, 90, 100, 200, 300, 400, 500, 600, 700, 800 or 900. In some embodiments, R³ is a PEG unit and r is about 2 to about 10 (e.g., r is about 2 to about 4). In some embodiments, s is about 5 to about 100, and sometimes s is about 5 to about 50 or about 5 to about 25. In certain embodiments, s is about 5 to about 15 and sometimes s is about 10. In some embodiments, s is about 5 or less (e.g., s is 1). In some embodiments, s can sometimes be, without limitation, about 2, 3, 4, 5, 6, 7, 8, 9, 10, 11, 12, 13, 14, 15, 16, 17, 18, 19, 20, 30, 40, 50, 60, 70, 80, 90, 100, 200, 300, 400, 500, 600, 700, 800 or 900. In some embodiments the (R³)ᵣ substituent is linear, and in certain embodiments, the (R³)ᵣ substituent is branched. For linear and branched moieties, r and s often are not equal, and sometimes r is less than s (e.g., branched PEG moiety) and at times s is less than r (e.g., linear PEG moiety).

In some embodiments, t is about 5 to about 100, and sometimes t is about 5 to about 50 or about 5 to about 25. In certain embodiments, t is about 5 to about 15 and sometimes t is about 10. In some embodiments, t is about 5 or less (e.g., t is. 1). In some embodiments, t can sometimes be, without limitation, about 2, 3, 4, 5, 6, 7, 8, 9, 10, 11, 12, 13, 14, 15, 16, 17, 18, 19, 20, 30, 40, 50, 60, 70, 80, 90, 100, 200, 300, 400, 500, 600, 700, 800 or 900. In certain embodiments, u is about 5 to about 100, and sometimes u is about 5 to about 50 or about 5 to about 25. In some embodiments, u is about 5 to about 15 and sometimes u is about 10. In certain embodiments, u is about 5 or less (e.g., u is 1). In some embodiments, u can sometimes be, without limitation, about 2, 3, 4, 5, 6, 7, 8, 9, 10, 11, 12, 13, 14, 15, 16, 17, 18, 19, 20, 30, 40, 50, 60, 70, 80, 90, 100, 200, 300, 400, 500, 600, 700, 800 or 900.

In certain embodiments, a R⁴ substituent independently is H, C1-C2 alkyl, —C1-C2 alkoxy (e.g., —OCH₃), —NR^{a}R^{b}, —OH, —CN, —COON, —COOR¹, —C1-C2 alkyl-NR^{a}R^{b}, C1-C2 alkyl-OH, C1-C2 alkyl-CN, C1-C2 alkyl-COOH or C1-C2 alkyl-COOR¹. In some embodiments, R⁴ is —R—CS—NR′R—. In some embodiments, R⁴ is an optionally substituted 5-6 membered ring (e.g., aryl ring, heteroaryl ring, carbocyclic ring, heterocyclic ring). In certain embodiments, R⁴ is not hydrogen, and sometimes R⁴ is not hydroxyl.

In some embodiments pertaining to a compound having a structure according to Formula I, m is about 1, R² is hydrogen and n is 4, q is 1, p is 1, r is about 10, and s is 1.

Each X⁴ can be the same macromolecule or a different macromolecule. In certain embodiments, a macromolecule is selected from the group consisting of an antibody, antibody fragment, antigen, pathogen antigen (e.g., S. aureus antigen), protein (e.g., human serum albumin protein or fragment thereof), glycerol, lipid, phospholipid (e.g., DOPE), sphingolipid and the like. In some embodiments, the macromolecule is DOPE.

In certain embodiments, X is N. In some embodiments, X¹ is O. In some embodiments, R is OH. In certain embodiments, m is 1, n is 0, p is 1, and q is 1. In some embodiments, X2 is a linking group. In certain embodiments, R³ is PEG. In certain embodiments, wherein r is 2 to 20, or r is 6 to 10. In certain embodiments, r is 6. In some embodiments, r is 10.

In certain embodiments, s is 3 and each R⁴ is selected from the group consisting of C1 to C6 alkyl, an optionally substituted 5 or 6-membered ring, and C1 to C6 alkyl COON. In some embodiments, X² is C(O)NH. In some embodiments, the 5 or 6-membered ring is substituted with N. In certain embodiments, the compound is a compound of Table 2. In some embodiments, the compound is Compound 2. In other embodiments, the compound is Compound 6.

In certain embodiments, the condition is a condition that may be treated by inducing macrophage M1 to M2 skewing. By skewing is meant shifting the activity from pro-inflammatory cells of an immune response to cells involved in tissue repair and remodeling. For example, the amount of at least one, two, three, four, or five M1 markers (TNF-alpha, IL12-p40, IL-1, IL6, CXCL10, and IFN-beta) is reduced and the amount of at least one, two, or three M2 markers (CCL17, CCL18, and CCL22) are increased after treatment, compared to before treatment. The percentage decrease or increase may be, for example, about 5, 10, 15, 20, 25, 30, 35, 40, 45, 50, 60, 70, 80, 90, or 100% or more. The decrease or increase may be measured by, for example, analysis of the amount of the marker, or, for example, the amount of an RNA transcript expressed in treated cells, that codes for the marker.

In certain embodiments, the condition is a fibrosis, inflammatory, or autoimmune condition. In certain embodiments, the condition is selected from the group consisting of lung fibrosis, Crohn's disease, cirrhosis, endomyocardial fibrosis, keloid, mediastinal fibrosis, myelofibrosis, myocardial infarction, nephrogenic systemic fibrosis, progressive massive fibrosis, pulmonary and idiopathic pulmonary fibrosis, retroperitoneal fibrosis, and scleroderma/systemic sclerosis. In some embodiments, the condition is lung fibrosis. In some embodiments, the condition is kidney disease.

In certain embodiments, the condition is selected from the group consisting of asthma, reactive airway disease, skin inflammation, rheumatoid arthritis. In some embodiments, the condition is asthma. In some embodiments, the condition is skin inflammation. In some embodiments, the condition is rheumatoid arthritis. In some embodiments, the condition is multiple sclerosis. In some embodiments, the condition is ulcerative colitis.

In certain embodiments, the subject is human. In some embodiments, the method further comprises administering an anti-inflammatory compound.

In an embodiment, the technology provides a method for preventing, inhibiting or treating a condition described herein in a subject, which comprises administering a compound having the following structure:

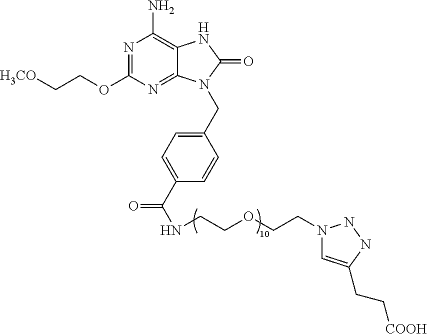

or a pharmaceutically acceptable salt thereof or hydrate thereof, to a human subject in need thereof in an amount effective to prevent, inhibit or treat the condition. In certain embodiments, the condition is lung fibrosis. In certain embodiments, the condition is rheumatoid arthritis. In certain embodiments, the condition is kidney failure.

In an embodiment, the technology provides a method for preventing, inhibiting or treating a condition described herein in a subject, which comprises administering a compound having the following structure:

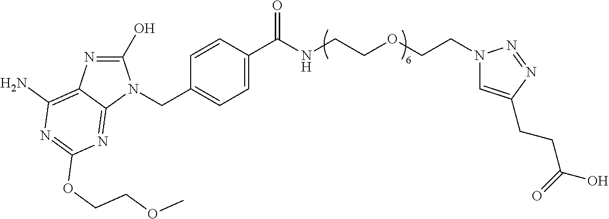

or a pharmaceutically acceptable salt thereof or hydrate thereof, to a human subject in need thereof in an amount effective to prevent, inhibit or treat the condition. In certain embodiments, the condition is rheumatoid arthritis. In certain embodiments, the condition is kidney failure.

In an embodiment, the technology provides a method for preventing, inhibiting or treating kidney failure in a subject, which comprises administering a compound having the following structure:

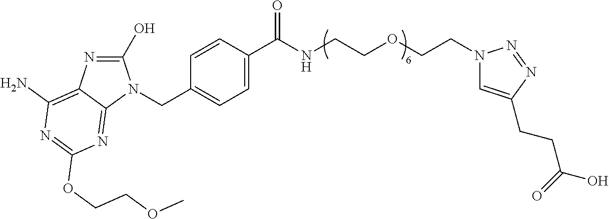

or a pharmaceutically acceptable salt thereof or hydrate thereof.

Thus, in certain embodiments provided herein are compounds for use in medical therapy, such as agents that prevent, inhibit, or treat the conditions described herein, optionally in conjunction with other compounds. Also provided in certain embodiments is the use of the compounds for the manufacture of a medicament to prevent, inhibit, or treat the conditions described herein.

Certain embodiments are described further in the following description, examples, claims and drawings.

### BRIEF DESCRIPTION OF THE DRAWINGS

The drawings illustrate embodiments of the technology and are not limiting. For clarity and ease of illustration, the drawings are not made to scale and, in some instances, various aspects may be shown exaggerated or enlarged to facilitate an understanding of particular embodiments.

FIG. 1. Prolonged exposure of murine peritoneal macrophages (PEC) to high concentration of TMX induced an M2 skewed activation. Results are given as fold increase over the mRNA level expressed by untreated cells (M/M) and are representative of 4 different experiments. M1 (▪) and M2 (□) genes. A and B, TMX was given at 10 microM; C and D, TMX was given at 1 microM. TMX=Compound 2.

FIG. 2. Expression of selected M1 and M2 gene products by TMX-tolerant murine peritoneal macrophages (PEC). To induce tolerance, PEC were pre-treated for 20 hours with 10 μM (A) or 1 μM (B) TMX. Next, supernatants from control (M/M), TMX activated (MIT), 1V209 activated (M/I), tolerant (T/M) and tolerant PEC re-challenged with TMX (T/T) or with 1V209 (T/I), were analyzed for the expression of representative M1 and M2 gene products by ELISA. Results are the average of three independent experiments, ±s. d. (*P_{<}0.01; t-test). M1 (▪) and M2 (□) genes.

FIG. 3. Analysis of cross-tolerance between TMX and TLR agonists. Murine peritoneal macrophages maintained in medium (black) or pre-treated for 20 hours with 10 μM (charcoal gray) or 1 μM (light gray) of TMX and re-challenged with the indicated TLR agonists were analyzed for the expression of selected M1 (A) and M2 (B) genes. Results are given as fold increase over the mRNA level expressed by untreated cells (M/M) and are representative of two different experiments.

FIG. 4. Analysis of cross-tolerance between TMX and the pro-inflammatory cytokine TNFα. Murine peritoneal macrophages maintained in medium (black) or pre-treated for 20 hours with 10 μM (charcoal gray) or 1 μM (light gray) of TMX and re-challenged with 20 ng/ml of TNFα were analyzed for the expression of selected M1 (A) and M2 (B) genes. Results are given as fold increase over the mRNA level expressed by untreated cells (M/M) and are representative of two different experiments.

FIG. 5. Expression of selected M1 and M2 gene transcripts in response to increased concentrations of TMX. Total RNA from murine peritoneal macrophages treated for 4 hours (▪) or 20 hours (□) with 100 ng/mlof LPS or increased concentrations of TMX or 1V209 were analyzed by RT PCR for the expression of representative M1 (TNFα, IL-12p40) and M2 (IL-10, TGFβ) genes. Results are given as fold increase over the mRNA level expressed by untreated cells (CTR).

FIG. 6. Analysis of NF-κB and STATs activity in TMX-tolerant macrophages. Nuclear and whole extracts from untreated (M/M), TMX-actiyated (M/T) for 90 min, tolerant (T/M), and tolerant peritoneal macrophages (PEC) rechallenged with TMX (T/T) were analyzed by Western blot for NF-κB and STATs members, respectively, as indicated. Equal loading is visualized by actin expression.

FIG. 7. Bleomycin-induced fibrosis was induced in C57BU6 (n=7) mice. WT mice were divided into four groups and groups 2 and 4 were treated with Compound 2 and groups 1 and 3 were treated with vehicle. Groups 1 and 2 mice were sacrificed on day 7, while groups 3 and 4 were terminated on day 21. BAL cells counts (A, B and C) and histological analysis (D-distribution, E-inflammation, and F-fibrosis) were performed as described in the protocol. The levels of IL-6 and IL-1b cytokines in BAL were under the detection levels of ELISA.

FIG. 8 presents data representing a dose-dependent reduction of paw thickness upon treatment with Compound 2.

FIG. 9. Analysis of M1 gene expression by human monocytes (Mo) treated with Compound 2. Results are presented as fold increase over the mRNA level expressed by untreated cells (M/M) and are obtained from 4 different donors (A=donor 1, B=donor 2, C=donor 3, D=donor 4).

FIG. 10. Analysis of M2 gene expression by human monocytes (Mo) treated with Compound 2. Results are given as fold increase over the mRNA level expressed by untreated cells (M/M) and are obtained from 4 different donors (A=donor 1, B=donor 2, C=donor 3, D=donor 4).

FIG. 11. Analysis of cross-tolerance between TMX and TLR agonists in terms of M1 gene expression. Mo maintained in medium (▪) or pre-treated for 20 hours with Compound 2 (□) and re-challenged with the indicated TLR agonists were analyzed for M1 gene expression. Results are given as fold increase over the mRNA level expressed by untreated cells and are obtained from 3 different donors (A=donor 2, B=donor 3, C=donor 4).

FIG. 12. Analysis of cross-tolerance between TMX and TLR agonists in terms of M2 gene expression. Mo maintained in medium (▪) or pre-treated for 20 hours with Compound 2 (□) and re-challenged with the indicated TLR agonists were analyzed for M2 gene expression. Results are given as fold increase over the mRNA level expressed by untreated cells and are obtained from 3 different donors (A=donor 2, B=donor 3, C=donor 4).

FIG. 13. Analysis of cross-tolerance between TMX and TLR agonists in terms of TNF-alpha production. Mo maintained in medium (▪) or pre-treated for 20 hours with Compound 2 (□) and re-challenged with the indicated TLR agonists were analyzed for TNF-alpha production. Results are obtained from 4 different donors (A=donor 5, B=donor 6, C=donor 7, D=donor 8).

FIG. 14. Analysis of cross-tolerance between TMX and TLR agonists in terms of IL-1beta production. Mo maintained in medium (▪) or pre-treated for 20 hours with Compound 2 (□) and re-challenged with the indicated TLR agonists were analyzed for IL1-beta production. Results are obtained from 4 different donors (A=donor 5, B=donor 6, C=donor 7, D=donor 8).

FIG. 15. Analysis of cross-tolerance between TMX and TLR agonists in terms of IL-6 production. Mo maintained in medium (▪) or pre-treated for 20 hours with Compound 2 (□) and re-challenged with the indicated TLR agonists were analyzed for IL-6 production. Results are obtained from 4 different donors (A=donor 5, B=donor 6, C=donor 7, D=donor 8).

FIG. 16. Analysis of cross-tolerance between TMX and TLR agonists in terms of IL-10 production. Mo maintained in medium (▪) or pre-treated for 20 hours with Compound 2 (□) and re-challenged with the indicated TLR agonists were analyzed for IL10 production. Results are obtained from 4 different donors (A=donor 5, B=donor 6, C=donor 7, D=donor 8).

FIG. 17. Analysis of cross-tolerance between TMX and TLR agonists in terms of CCL17 production. Mo maintained in medium (▪) or pre-treated for 20 hours with Compound 2 (□) and re-challenged with the indicated TLR agonists were analyzed for CCL17 production. Results are obtained from 4 different donors (A=donor 5, B=donor 6, C=donor 7, D=donor 8).

FIG. 18. Analysis of cross-tolerance between TMX and TLR agonist in terms of CCL22 production. Mo maintained in medium (▪) or pre-treated for 20 hours with Compound 2 (□) and re-challenged with the indicated TLR agonists were analyzed for CCL22 production. Results are obtained from 2 different donors (A=donor 7, B=donor 8).

FIG. 19 provides examples of synthesis schemes that may be used to synthesize certain compounds described herein.

### DETAILED DESCRIPTION

### Compounds

Small molecule TLR modulators are known. Examples of small molecules are described in U.S. Pat. No. 6,329,381, issued on Dec. 11, 2001 from patent application Ser. No. 09/555,292 filed on May 26, 2000, and in PCT/US2006/032371, filed on Aug. 21, 2006 (published as WO2007/024707 on Mar. 1, 2007); PCT/US2008/001631, filed on Feb. 7, 2008 (published as WO 2008/115319 on Sep. 25, 2008); PCT/US07/009,840, filed on Apr. 23, 2007 (published as WO/2007/142755 on Dec. 13, 2007); U.S. patent application Ser. No. 12/367,172 filed on Feb. 6, 2009, and PCT Application No. PCT/US09/00771 filed on Feb. 6, 2009, published as WO 2009/099650, on Aug. 13, 2009.

It has been determined that certain small molecule TLR agonists (e.g., sometimes referred to as a “small molecule target” herein) can be conjugated to one or more PEG moieties, and the resulting conjugate can exhibit TLR antagonist activity. There are several methods known for conjugating a small molecule target to one or more PEG moieties. For example, several PEG reactants are commercially available and are suitable for conjugation to a variety of reactive groups on the small molecule (e.g., NOF Corporation, Japan (World Wide Web URL: peg-drug.com/peg_product/activated_peg.html)). The term “PEG reactant” as used herein refers to a molecule that is combined with a small molecule target under conditions that generate a PEG-small molecule target conjugate product. For example, certain PEG reactants having the following structure can react with a variety of target groups on a small molecule:

CH₃O(CH₂CH₂O)ₙ—X, where X is a reactive group according to Table 1, and n equals “r” defined above for Formula I, II or III, in some embodiments:

TABLE 1

| | Reactive Group on Small |
|---|---|
| Reactive Group | Molecule Target |
| —CO—CH₂CH₂—COO—NHS* | —NH₂, —OH, —SH |
| —CO—CH₂CH₂CH₂—COO—NHS* | —NH₂, —OH, —SH |
| —CH₂—COO—NHS* | —NH₂, —OH, —SH |
| —CH₂CH₂CH₂CH₂CH₂—COO—NHS* | —NH₂, —OH, —SH |
| —CO₂-p-C₆H₄—NO₂ | —NH₂ |
| —CH₂CH₂—CHO | —NH₂ |
| —CH₂CH₂CH₂NH₂ | —COOH |
| —CH₂CH₂CH(OC₂H₅)₂ | —NH₂ |
| —CH₂CH₂SH— | SH, —N-Maleimidyl, |
| | —COOH |
| —CH₂CH₂CH₂NHCOCH₂CH₂—N- | —SH |
| Maleimidyl | |

and where NHS* is N-Succinimidyl.

In some embodiments, a PEG reactant has a structure CH₃O(CH₂CH₂O)ₙ—X—NHS*, where X can be —COCH₂CH₂COO—, —COCH₂CH₂CH₂COO—, —CH₂COO—, and —(CH₂)₅COO, and n equals “r” defined above for Formula I, II or III, in some embodiments. In certain embodiments, a PEG reactant has a structure

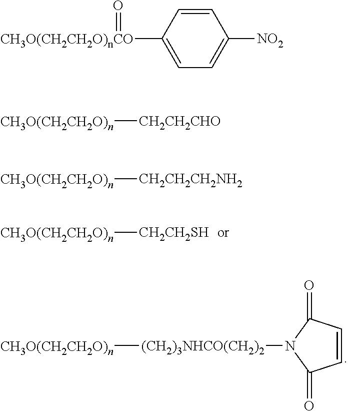

In the five structures above, designated structures I-V, respectively, substituent “n” shown in the PEG reactants, only, equals “r” defined above for Formula I, II or III, in some embodiments.

Certain PEG reactants are bifunctional in some embodiments. Examples of bifunctional PEG reactants have a structure X—(OCH₂CH₂)n-X, where X is (N-Succinimidyloxycarbonyl)methyl (—CH₂COO—NHS), Succinimidylglutarate (—COCH₂CH₂CH₂COO—NHS), (N-Succinimidyloxycarbonyl)pentyl (—(CH₂)₅COO—NHS), 3-(N-Maleimidyl)propanamido, (—NHCOCH₂CH₂-MAL), Aminopropyl (—CH₂CH₂CH₂NH₂) or 2-Sulfanylethyl (—CH₂CH₂SH) in some embodiments, where n equals “r” defined above for Formula I, II or III, in some embodiments

In certain embodiments, some PEG reactants are heterofunctional. Examples of heterofunctional PEG reactants have the structures

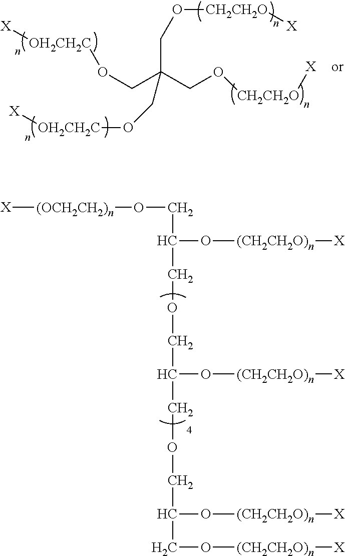

where X can be (N-Succinimidyloxycarbonyl)methyl (—CH₂COO—NHS), Succinimidylglutarate (—COCH₂CH₂CH₂COO—NHS), (N-Succinimidyloxycarbonyl)pentyl (—(CH₂)₅COO—NHS), 3-(N-Maleimidyl)propanamido, (—NHCOCH₂CH₂-MAL), 3-aminopropyl (—CH₂CH₂CH₂NH₂), 2-Sulfanylethyl (—CH₂CH₂SH), 5-(N-Succinimidyloxycarbonyl)pentyl (—(CH₂)₅COO—NHS], or p-Nitrophenyloxycarbonyl, (—CO₂-p-C₆H₄NO₂), in some embodiments.

In the two structures above, designated structures VI and VII, respectively, substituent “n” shown in the PEG reactants, only, equals “r” defined above for Formula I, II or III, in some embodiments.

Certain branched PEG reactants also may be utilized, such as those having a structure VIII:

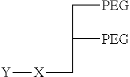

where X is a spacer and Y is a functional group, including, but not limited to, maleimide, amine, glutaryl-NHS, carbonate-NHS or carbonate-p-nitrophenol, in some embodiments. An advantage of branched chain PEG reactants is that they can yield conjugation products that have sustained release properties.

A PEG reactant also may be a heterofunctional reactant, such as

HO(CH₂CH₂O)ₙ—CH₂CH₂CH₂NH₂

HCl.H₂N—CH₂CH₂CH₂O(CH₂CH₂O)ₙ—(CH₂)₅COOH

and

HO(CH₂CH₂O)ₙ—CH₂CH₂CHO

in certain embodiments. In some embodiments, Boc*-protected-Amino-PEG-Carboxyl-NHS or Maleimide-PEG-Carboxyl-NHS reactants can be utilized.

In the three structures above, designated structures IX, X, and XI, respectively, substituent “n” shown in the PEG reactants, only, equals “r” defined above for Formula I, II or III, in some embodiments.

In certain embodiments, a comb-shaped polymer may be utilized as a PEG reactant to incorporate a number of PEG units into a conjugate. An example of a comb-shaped polymer is shown hereafter.

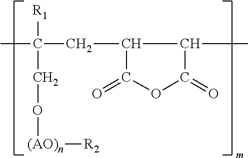

In the structure above, designated structure XII, substituents “m” and “n” shown in the PEG reactants, only, equals “r” defined above for Formula I, II or III, in some embodiments.

A PEG reactant, and/or a PEG conjugate product, can have a molecular weight ranging between about 5 grams per mole to about 100,000 grams per mole. In some embodiments, a PEG reactant, and/or a PEG conjugate product, has a average, mean or nominal molecular weight of about 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000 or 90000 grams per mole. In some embodiments the PEG moiety in a compound herein is homogeneous and the molecule weight of the PEG moiety is the same for each molecule of a particular batch of compound (e.g., R³ is one PEG unit and r is 2 to 10).

In certain embodiments, one or more R⁴ substituents terminate the PEG moiety (e.g., Formula I; linear or branched PEG moiety). Each R⁴ substituent may be the same or different, and can be selected independently from the group consisting of —CH₂—CH₂—NH₂, —CH₂—CH₂—OH, —CH₂—CH₂—COOH, —CH₂—CH₂—COOR¹, in some embodiments. The linker can be any suitable linker, including a linker described herein.

A suitable linker can be utilized to construct conjugates (e.g., X², X³), and multiple linkers are known. Non-limiting examples of linkers that can be utilized include the following:

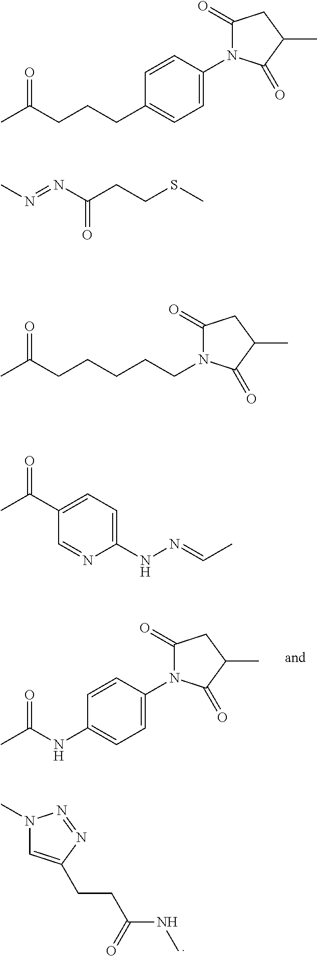

As used herein, the terms “alkyl,” “alkenyl” and “alkynyl” include straight-chain, branched-chain and cyclic monovalent hydrocarbyl radicals, and combinations of these, which contain only C and H when they are unsubstituted. Examples include methyl, ethyl, isobutyl, cyclohexyl, cyclopentylethyl, 2 propenyl, 3 butynyl, and the like. The total number of carbon atoms in each such group is sometimes described herein, e.g., when the group can contain up to ten carbon atoms it can be represented as 1-10C or as C1-C10 or C1-10. When heteroatoms (N, O and S typically) are allowed to replace carbon atoms as in heteroalkyl groups, for example, the numbers describing the group, though still written as e.g. C1-C6, represent the sum of the number of carbon atoms in the group plus the number of such heteroatoms that are included as replacements for carbon atoms in the backbone of the ring or chain being described.

Typically, the alkyl, alkenyl and alkynyl substituents of the technology contain one 10C (alkyl) or two 10C (alkenyl or alkynyl). They may, for example contain one 8C (alkyl) or two 8C (alkenyl or alkynyl). Sometimes they contain one 4C (alkyl) or two 4C (alkenyl or alkynyl). A single group can include more than one type of multiple bond, or more than one multiple bond; such groups are included within the definition of the term “alkenyl” when they contain at least one carbon-carbon double bond, and are included within the term “alkynyl” when they contain at least one carbon-carbon triple bond.

Alkyl, alkenyl and alkynyl groups are often optionally substituted to the extent that such substitution makes sense chemically. Typical substituents include, but are not limited to, halo, ═O, ═N—CN, ═N—OR, ═NR, OR, NR₂, SR, SO₂R, SO₂NR₂, NRSO₂R, NRCONR₂, NRCOOR, NRCOR, CN, COOR, CONR₂, OOCR, COR, and NO₂, wherein each R is independently H, C1-C8 alkyl, C2-C8 heteroalkyl, C1-C8 acyl, C2-C8 heteroacyl, C2-C8 alkenyl, C2-C8 heteroalkenyl, C2-C8 alkynyl, C2-C8 heteroalkynyl, C6-C10 aryl, or C5-C10 heteroaryl, and each R is optionally substituted with halo, ═O, ═N—CN, ═N—OR′, ═NR′, OR′, NR′₂, SR′, SO₂R′, SO₂NR′₂, NR′SO₂R′, NR′CONR′₂, NR′COOR′, NR′COR′, CN, COOR′, CONR′₂, OOCR′, COR′, and NO₂, wherein each R′ is independently H, C1-C8 alkyl, C2-C8 heteroalkyl, C1-C8 acyl, C2-C8 heteroacyl, C6-C10 aryl or C5-C10 heteroaryl. Alkyl, alkenyl and alkynyl groups can also be substituted by C1-C8 acyl, C2-C8 heteroacyl, C6-C10 aryl or C5-C10 heteroaryl, each of which can be substituted by the substituents that are appropriate for the particular group.

“Acetylene” substituents are 2-10C alkynyl groups that are optionally substituted, and are of the formula —CEC-Ri, wherein Ri is H or C1-C8 alkyl, C2-C8 heteroalkyl, C2-C8 alkenyl, C2-C8 heteroalkenyl, C2-C8 alkynyl, C2-C8 heteroalkynyl, C1-C8 acyl, C2-C8 heteroacyl, C6-C10 aryl, C5-C10 heteroaryl, C7-C12 arylalkyl, or C6-C12 heteroarylalkyl, and each Ri group is optionally substituted with one or more substituents selected from halo, ═O, ═N—CN, ═N—OR′, ═NR′, OR′, NR′2, SR′, SO₂R′, SO₂NR′₂, NR′SO₂R′, NR′CONR′₂, NR′COOR′, NR′COR′, CN, COOR′, CONR′₂, OOCR′, COR′, and NO₂, wherein each R′ is independently H, C1-C6 alkyl, C2-C6 heteroalkyl, C1-C6 acyl, C2-C6 heteroacyl, C6-C10 aryl, C5-C10 heteroaryl, C7-12 arylalkyl, or C6-12 heteroarylalkyl, each of which is optionally substituted with one or more groups selected from halo, C1-C4 alkyl, C1-C4 heteroalkyl, C1-C6 acyl, C1-C6 heteroacyl, hydroxy, amino, and ═O; and wherein two R′ can be linked to form a 3-7 membered ring optionally containing up to three heteroatoms selected from N, O and S. In some embodiments, Ri of —C≡C—Ri is H or Me.

“Heteroalkyl”, “heteroalkenyl”, and “heteroalkynyl” and the like are defined similarly to the corresponding hydrocarbyl (alkyl, alkenyl and alkynyl) groups, but the ‘hetero’ terms refer to groups that contain one to three O, S or N heteroatoms or combinations thereof within the backbone residue; thus at least one carbon atom of a corresponding alkyl, alkenyl, or alkynyl group is replaced by one of the specified heteroatoms to form a heteroalkyl, heteroalkenyl, or heteroalkynyl group. The typical sizes for heteroforms of alkyl, alkenyl and alkynyl groups are generally the same as for the corresponding hydrocarbyl groups, and the substituents that may be present on the heteroforms are the same as those described above for the hydrocarbyl groups. For reasons of chemical stability, it is also understood that, unless otherwise specified, such groups do not include more than two contiguous heteroatoms except where an oxo group is present on N or S as in a nitro or sulfonyl group.

While “alkyl” as used herein includes cycloalkyl and cycloalkylalkyl groups, the term “cycloalkyl” may be used herein to describe a carbocyclic non-aromatic group that is connected via a ring carbon atom, and “cycloalkylalkyl” may be used to describe a carbocyclic non-aromatic group that is connected to the molecule through an alkyl linker. Similarly, “heterocyclyl” may be used to describe a non-aromatic cyclic group that contains at least one heteroatom as a ring member and that is connected to the molecule via a ring atom, which may be C or N; and “heterocyclylalkyl” may be used to describe such a group that is connected to another molecule through a linker. The sizes and substituents that are suitable for the cycloalkyl, cycloalkylalkyl, heterocyclyl, and heterocyclylalkyl groups are the same as those described above for alkyl groups. As used herein, these terms also include rings that contain a double bond or two, as long as the ring is not aromatic.

As used herein, “acyl” encompasses groups comprising an alkyl, alkenyl, alkynyl, aryl or arylalkyl radical attached at one of the two available valence positions of a carbonyl carbon atom, and heteroacyl refers to the corresponding groups wherein at least one carbon other than the carbonyl carbon has been replaced by a heteroatom chosen from N, O and S. Thus heteroacyl includes, for example, —C(═O)OR and —C(═O)NR₂ as well as —C(═O)-heteroaryl.

Acyl and heteroacyl groups are bonded to any group or molecule to which they are attached through the open valence of the carbonyl carbon atom. Typically, they are C1-C8 acyl groups, which include formyl, acetyl, pivaloyl, and benzoyl, and C2-C8 heteroacyl groups, which include methoxyacetyl, ethoxycarbonyl, and 4-pyridinoyl. The hydrocarbyl groups, aryl groups, and heteroforms of such groups that comprise an acyl or heteroacyl group can be substituted with the substituents described herein as generally suitable substituents for each of the corresponding component of the acyl or heteroacyl group.

“Aromatic” moiety or “aryl” moiety refers to a monocyclic or fused bicyclic moiety having the well-known characteristics of aromaticity; examples include phenyl and naphthyl. Similarly, “heteroaromatic” and “heteroaryl” refer to such monocyclic or fused bicyclic ring systems which contain as ring members one or more heteroatoms selected from O, S and N. The inclusion of a heteroatom permits aromaticity in 5 membered rings as well as 6 membered rings. Typical heteroaromatic systems include monocyclic C5-C6 aromatic groups such as pyridyl, pyrimidyl, pyrazinyl, thienyl, furanyl, pyrrolyl, pyrazolyl, thiazolyl, oxazolyl, and imidazolyl and the fused bicyclic moieties formed by fusing one of these monocyclic groups with a phenyl ring or with any of the heteroaromatic monocyclic groups to form a C8-C10 bicyclic group such as indolyl, benzimidazolyl, indazolyl, benzotriazolyl, isoquinolyl, quinolyl, benzothiazolyl, benzofuranyl, pyrazolopyridyl, quinazolinyl, quinoxalinyl, cinnolinyl, and the like. Any monocyclic or fused ring bicyclic system which has the characteristics of aromaticity in terms of electron distribution throughout the ring system is included in this definition. It also includes bicyclic groups where at least the ring which is directly attached to the remainder of the molecule has the characteristics of aromaticity. Typically, the ring systems contain 5-12 ring member atoms: The monocyclic heteroaryls may, for example, contain 5-6 ring members, and the bicyclic heteroaryls contain 8-10 ring members.

Aryl and heteroaryl moieties may be substituted with a variety of substituents including C1-C8 alkyl, C2-C8 alkenyl, C2-C8 alkynyl, C5-C12 aryl, C1-C8 acyl, and heteroforms of these, each of which can itself be further substituted; other substituents for aryl and heteroaryl moieties include halo, OR, NR₂, SR, SO₂R, SO₂NR₂, NRSO₂R, NRCONR₂, NRCOOR, NRCOR, CN, COOR, CONR₂, OOCR, COR, and NO₂, wherein each R is independently H, C1-C8 alkyl, C2-C8 heteroalkyl, C2-C8 alkenyl, C2-C8 heteroalkenyl, C2-C8 alkynyl, C2-C8 heteroalkynyl, C6-C10 aryl, C5-C10 heteroaryl, C7-C12 arylalkyl, or C6-C12 heteroarylalkyl, and each R is optionally substituted as described above for alkyl groups. The substituent groups on an aryl or heteroaryl group may of course be further substituted with the groups described herein as suitable for each type of such substituents or for each component of the substituent. Thus, for example, an arylalkyl substituent may be substituted on the aryl portion with substituents described herein as typical for aryl groups, and it may be further substituted on the alkyl portion with substituents described herein as typical or suitable for alkyl groups.

Similarly, “arylalkyl” and “heteroarylalkyl” refer to aromatic and heteroaromatic ring systems which are bonded to their attachment point through a linking group such as an alkylene, including substituted or unsubstituted, saturated or unsaturated, cyclic or acyclic linkers. Typically the linker is C1-C8 alkyl or a hetero form thereof. These linkers may also include a carbonyl group, thus making them able to provide substituents as an acyl or heteroacyl moiety. An aryl or heteroaryl ring in an arylalkyl or heteroarylalkyl group may be substituted with the same substituents described above for aryl groups. An arylalkyl group may, for example, include a phenyl ring optionally substituted with the groups defined above for aryl groups and a C1-C4 alkylene that is unsubstituted or is substituted with one or two C1-C4 alkyl groups or heteroalkyl groups, where the alkyl or heteroalkyl groups can optionally cyclize to form a ring such as cyclopropane, dioxolane, or oxacyclopentane. Similarly, a heteroarylalkyl group may, for example, include a C5-C6 monocyclic heteroaryl group that is optionally substituted with the groups described above as substituents typical on aryl groups and a C1-C4 alkylene that is unsubstituted or is substituted with one or two C1-C4 alkyl groups or heteroalkyl groups, or it includes an optionally substituted phenyl ring or C5-C6 monocyclic heteroaryl and a C1-C4 heteroalkylene that is unsubstituted or is substituted with one or two C1-C4 alkyl or heteroalkyl groups, where the alkyl or heteroalkyl groups can optionally cyclize to form a ring such as cyclopropane, dioxolane, or oxacyclopentane.

Where an arylalkyl or heteroarylalkyl group is described as optionally substituted, the substituents may be on either the alkyl or heteroalkyl portion or on the aryl or heteroaryl portion of the group. The substituents optionally present on the alkyl or heteroalkyl portion are the same as those described above for alkyl groups generally; the substituents optionally present on the aryl or heteroaryl portion are the same as those described above for aryl groups generally.

“Arylalkyl” groups as used herein are hydrocarbyl groups if they are unsubstituted, and are described by the total number of carbon atoms in the ring and alkylene or similar linker. Thus a benzyl group is a C7-arylalkyl group, and phenylethyl is a C8-arylalkyl.

“Heteroarylalkyl” as described above refers to a moiety comprising an aryl group that is attached through a linking group, and differs from “arylalkyl” in that at least one ring atom of the aryl moiety or one atom in the linking group is a heteroatom selected from N, O and S. The heteroarylalkyl groups are described herein according to the total number of atoms in the ring and linker combined, and they include aryl groups linked through a heteroalkyl linker; heteroaryl groups linked through a hydrocarbyl linker such as an alkylene; and heteroaryl groups linked through a heteroalkyl linker. Thus, for example, C7-heteroarylalkyl would include pyridylmethyl, phenoxy, and N-pyrrolylmethoxy.

“Alkylene” as used herein refers to a divalent hydrocarbyl group; because it is divalent, it can link two other groups together. Typically it refers to —(CH₂)ₙ— where n is 1-8 and, for example, n may be 1-4, though where specified, an alkylene can also be substituted by other groups, and can be of other lengths, and the open valences need not be at opposite ends of a chain. Thus —CH(Me)— and —C(Me)₂— may also be referred to as alkylenes, as can a cyclic group such as cyclopropan-1,1-diyl. Where an alkylene group is substituted, the substituents include those typically present on alkyl groups as described herein.

In general, any alkyl, alkenyl, alkynyl, acyl, or aryl or arylalkyl group or any heteroform of one of these groups that is contained in a substituent may itself optionally be substituted by additional substituents. The nature of these substituents is similar to those recited with regard to the primary substituents themselves if the substituents are not otherwise described. Thus, where an embodiment of, for example, R2 is alkyl, this alkyl may optionally be substituted by the remaining substituents listed as embodiments for R2 where this makes chemical sense, and where this does not undermine the size limit provided for the alkyl per se; e.g., alkyl substituted by alkyl or by alkenyl would simply extend the upper limit of carbon atoms for these embodiments, and is not included. However, alkyl substituted by aryl, amino, alkoxy, ═O, and the like would be included within the scope of the technology, and the atoms of these substituent groups are not counted in the number used to describe the alkyl, alkenyl, etc. group that is being described. Where no number of substituents is specified, each such alkyl, alkenyl, alkynyl, acyl, or aryl group may be substituted with a number of substituents according to its available valences; in particular, any of these groups may be substituted with fluorine atoms at any or all of its available valences, for example.

“Heteroform” as used herein refers to a derivative of a group such as an alkyl, aryl, or acyl, wherein at least one carbon atom of the designated carbocyclic group has been replaced by a heteroatom selected from N, O and S. Thus the heteroforms of alkyl, alkenyl, alkynyl, acyl, aryl, and arylalkyl are heteroalkyl, heteroalkenyl, heteroalkynyl, heteroacyl, heteroaryl, and heteroarylalkyl, respectively. It is understood that no more than two N, O or S atoms are ordinarily connected sequentially, except where an oxo group is attached to N or S to form a nitro or sulfonyl group. A heteroform moiety sometimes is referred to as “Het” herein.

“Halo” or “halogen,” as used herein includes fluoro, chloro, bromo and iodo. Fluoro and chloro are often typical. “Amino” as used herein refers to NH₂, but where an amino is described as “substituted” or “optionally substituted”, the term includes NR′R″ wherein each R′ and R″ is independently H, or is an alkyl, alkenyl, alkynyl, acyl, aryl, or arylalkyl group or a heteroform of one of these groups, and each of the alkyl, alkenyl, alkynyl, acyl, aryl, or arylalkyl groups or heteroforms of one of these groups is optionally substituted with the substituents described herein as suitable for the corresponding group. The term also includes forms wherein R′ and R″ are linked together to form a 3-8 membered ring which may be saturated, unsaturated or aromatic and which contains 1-3 heteroatoms independently selected from N, O and S as ring members, and which is optionally substituted with the substituents described as suitable for alkyl groups or, if NR′R″ is an aromatic group, it is optionally substituted with the substituents described as typical for heteroaryl groups.

As used herein, the term “carbocycle” refers to a cyclic compound containing only carbon atoms in the ring, whereas a “heterocycle” refers to a cyclic compound comprising a heteroatom. The carbocyclic and heterocyclic structures encompass compounds having monocyclic, bicyclic or multiple ring systems. As used herein, the term “heteroatom” refers to any atom that is not carbon or hydrogen, such as nitrogen, oxygen or sulfur. Illustrative examples of heterocycles include but are not limited to tetrahydrofuran, 1,3dioxolane, 2,3dihydrofuran, pyran, tetrahydropyran, benzofuran, isobenzofuran, 1,3dihydro isobenzofuran, isoxazole, 4,5dihydroisoxazole, piperidine, pyrrolidine, pyrrolidin 2 one, pyrrole, pyridine, pyrimidine, octahydro pyrrolo[3,4b]pyridine, piperazine, pyrazine, morpholine, thiomorpholine, imidazole, imidazolidine 2,4dione, 1,3dihydrobenzimidazol 2 one, indole, thiazole, benzothiazole, thiadiazole, thiophene, tetrahydro thiophene 1,1 dioxide, diazepine, triazole, guanidine, diazabicyclo[2.2.1]heptane, 2,5 diazabicyclo[2.2.1]heptane, 2,3,4,4a,9,9a hexahydro 1H beta carboline, oxirane, oxetane, tetrahydropyran, dioxane, lactones, aziridine, azetidine, piperidine, lactams, and may also encompass heteroaryls. Other illustrative examples of heteroaryls include but are not limited to furan, pyrrole, pyridine, pyrimidine, imidazole, benzimidazole and triazole.

As used herein, a linking group may be an amido linking group (e.g. —C(O)NH— or —NH(O)C—); alkyl amido linking group (e.g., —C1-C6 alkyl-C(O)NH—, —C1-C6 alkyl-NH(O)C—, —C(O)NH—C1-C6 alkyl-, —NH(O)C—C1-C6 alkyl-, —C1-C6 alkyl-NH(O)C—C1-C6 alkyl-, —C1-C6 alkyl-C(O)NH—C1-C6 alkyl-), or thioamide R—CS—NR′R.

In some cases, certain compounds described herein contain one or more chiral centers. The technology includes each of the isolated stereoisomeric forms as well as mixtures of stereoisomers in varying degrees of chiral purity, including racemic mixtures. It also encompasses the various diastereomers and tautomers that can be formed. The compounds of the technology may also exist in one or more tautomeric forms. For example, when R is —OH, a compound described herein may exist in one or more tautomeric forms.

The term “optionally substituted” as used herein indicates that the particular group or groups being described may have no non-hydrogen substituents, or the group or groups may have one or more non-hydrogen substituents. If not otherwise specified, the total number of such substituents that may be present is equal to the number of H atoms present on the unsubstituted form of the group being described. Where an optional substituent is attached via a double bond, such as a carbonyl oxygen (═O), the group takes up two available valences, so the total number of substituents that may be included is reduced according to the number of available valences.

### Pharmaceutical Compositions and Formulations

A compound described herein can be prepared as a pharmaceutically acceptable salt. As used herein, the term “pharmaceutically acceptable salt” refers to a derivative of the disclosed compounds where the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. Pharmaceutically acceptable salts include conventional non-toxic salts or quaternary ammonium salts of the parent compound formed, for example, from non-toxic inorganic or organic acids. For example, conventional non-toxic salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like. In other examples, conventional non-toxic salts include those derived from bases, such as potassium hydroxide, sodium hydroxide, ammonium hydroxide, caffeine, various amines, and the like. Pharmaceutically acceptable salts can be synthesized from the parent compound, which contains a basic or acidic moiety, by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are typical. Lists of suitable salts are found in Remington's Pharmaceutical Sciences, 17th ed., Mack Publishing Company, Easton, Pa., p. 1418 (1985), the disclosure of which is hereby incorporated by reference.

The term “pharmaceutically acceptable” as used herein refers to compounds, materials, compositions, and/or dosage forms which are, within the scope of sound medical judgment, suitable for use in contact with the tissues of human beings and animals without excessive toxicity, irritation, allergic response, or other problem or complication commensurate with a reasonable benefit/risk ratio.

The terms “stable compound” and “stable structure” are meant to indicate a compound that is sufficiently robust to survive isolation to a useful degree of purity from a reaction mixture, and formulation into an efficacious therapeutic agent. Stable compounds are contemplated herein for use in treatment methods described.

A compound described herein can be formulated in combination with one or more other agents. The one or more other agents can include, without limitation, another compound described herein, an anti-cell proliferative agent (e.g., chemotherapeutic), an anti-inflammatory agent, or an antigen.

A compound described herein can be formulated as a pharmaceutical composition and administered to a mammalian host, such as a human patient or nonhuman animal, in a variety of forms adapted to the chosen route of administration, e.g., orally or parenterally, by intravenous, intramuscular, topical or subcutaneous routes. In certain embodiments, a composition is locally administered, e.g., intravesicularly. A composition often includes a diluent as well as, in some cases, an adjuvant, buffer, preservative and the like. A compound can be administered also in a liposomal composition or as a microemulsion, in certain embodiments. Various sustained release systems for drugs have also been devised, and can be applied to a compound described herein. See, for example, U.S. Pat. No. 5,624,677, the methods of which are incorporated herein by reference.

Thus, compounds may be systemically administered, e.g., orally, in combination with a pharmaceutically acceptable vehicle such as an inert diluent or an assimilable edible carrier.

Certain compounds described herein may be enclosed in hard or soft shell gelatin capsules, may be compressed into tablets, or may be incorporated directly with the food of the patient's diet. For oral therapeutic administration, the active compound may be combined with one or more excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 60% of the weight of a given unit dosage form. The amount of active compound in such therapeutically useful compositions is such that an effective dosage level will be obtained.

Tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations and devices.

An active compound may be administered by infusion or injection. Solutions of an active compound or a pharmaceutically acceptable salt thereof can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations may contain a preservative to prevent the growth of microorganisms.

A pharmaceutical dosage form can include a sterile aqueous solution or dispersion or sterile powder comprising an active ingredient, which is adapted for the extemporaneous preparation of sterile solutions or dispersions, and optionally encapsulated in liposomes. The ultimate dosage form sometimes is a sterile fluid and stable under the conditions of manufacture and storage. A liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. An isotonic agent, for example, a sugar, buffer or sodium chloride is included in some embodiments. Prolonged absorption of an injectable composition can be brought about by the use in the compositions of agents delaying absorption, for example, aluminum monostearate and gelatin. Sterile solutions often are prepared by incorporating an active compound in a required amount in an appropriate solvent, sometimes with one or more of the other ingredients enumerated above, followed by filter sterilization. In the case of sterile powders for the preparation of sterile injectable solutions, preparation methods sometimes utilized are vacuum drying and the freeze drying techniques, which yield a powder of an active ingredient in addition to any additional desired ingredient present in the previously sterile-filtered solutions.

For topical administration, a compound herein may be applied in pure form, e.g., when in liquid form. However, it is generally desirable to administer a compound as a composition or formulation, in combination with an acceptable carrier, which may be a solid or a liquid. Useful solid carriers include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user. Useful dosages of the compounds can be determined by comparing their in vitro activity, and in vivo activity in animal models. Methods for the extrapolation of effective dosages in mice, and other animals, to humans are known to the art; for example, see U.S. Pat. No. 4,938,949. The ability of a compound herein to act as a TLR agonist or TLR antagonist may be determined using pharmacological models which are known, including the procedures disclosed by Lee et al., PNAS, 100:6646 (2003). Generally, the concentration of the compound(s) in a liquid composition will be from about 0.1-25 wt-%, for example from about 0.5-10 wt-%. The concentration in a semi-solid or solid composition such as a gel or a powder will be about 0.1-5 wt-%, for example, about 0.5-2.5 wt-%.

### Treatments

Compositions provided may be useful for the treatment or prevention of certain conditions in a subject. Such conditions include, for example, fibrosis, autoimmune, and inflammatory conditions. in certain embodiments.

The terms “treat” and “treating” as used herein refer to (i) preventing a pathologic condition from occurring (e.g. prophylaxis); (ii) inhibiting the pathologic condition or arresting its development; (iii) relieving the pathologic condition; and/or (iv) ameliorating, alleviating, lessening, and removing symptoms of a disease or condition. A candidate molecule or compound described herein may be in a therapeutically effective amount in a formulation or medicament, which is an amount that can lead to a biological effect (e.g., inhibiting inflammation), or lead to ameliorating, alleviating, lessening, relieving, diminishing or removing symptoms of a disease or condition, for example.

The term “therapeutically effective amount” as used herein refers to an amount of a compound provided herein, or an amount of a combination of compounds provided herein, to treat or prevent a disease or disorder, or to treat a symptom of the disease or disorder, in a subject. As used herein, the terms “subject” and “patient” generally refers to an individual who will receive or who has received treatment (e.g., administration of a compound described herein) according to a method described herein.

A drug, which can be a prophylactic or therapeutic agent, can be administered to any appropriate subject having a condition as described herein. Non-limiting examples of a subject include mammal, human, ape, monkey, ungulate (e.g., equine, bovine, caprine, ovine, porcine, buffalo, camel and the like), canine, feline, rodent (e.g., murine, mouse, rat) and the like. A subject may be male or female, and a drug can be administered to a subject in a particular age group, including, for example, juvenile, pediatric, adolescent, adult and the like.

Examples of conditions that may be treated by inducing macrophage M1 to M2 skewing, and may be treated by administering a compound described herein to a subject, include, but are not limited to, a fibrosis condition (e.g., Crohn's disease, cirrhosis, renal fibrosis, endomyocardial fibrosis, keloid, mediastinal fibrosis, myelofibrosis, myocardial infarction, nephrogenic systemic fibrosis, progressive massive fibrosis, pulmonary and idiopathic pulmonary fibrosis, retroperitoneal fibrosis, lung fibrosis, sarcoidosis, scleroderma/systemic sclerosis, multiple sclerosis); a condition requiring tissue repair, a tissue remodeling and/or wound healing condition, ulcerative colitis; a liver failure condition (e.g., characterized by a variation in a level of one or more liver blood enzymes such as aminotransferases for example asparatate and alaninie transferases, alkaline phoshatase, 5′-nucleotidase, and gamma glutamyltranspeptidase); an inflammatory or autoimmune condition or disorder (e.g. asthma, reactive airway disease, skin inflammation, rheumatoid arthritis), a kidney failure condition (e.g., characterized by a variation in a level of one or more of creatinine, blood urea-nitrogen, red blood cells, white blood cells, leukocytes, protein, microalbumin, parathyroid hormone, and/or cystatin C); a lung damage condition (e.g., characterized by a variation in a level of one or more of lactate dehydrogenase, isoenzymes, glucose-6-phosphate-dehydrorgenase, lysosomal acid hydrolases, alkaline phosphatase, glutathione peroxidase/reductase, angiotension converting enzyme, sialic acid and phagocytic cells); an atherosclerosis or vascular condition (e.g., characterized by a variation in a level of one or more of lipoproteins, apolipoproteins, and/or glycosaminoglycan); an inflammatory bowel condition (e.g., characterized by a variation in a level of one or more of anti-neutrophil cytoplasmic autoantibodies, anti-Saccharomyces cerevisiae antibodies, Escherichia coli-related OmpC, pseudomonas fluorescens and flagellin CBir1); a post surgical adhesion condition; a peritoneal adhesion condition (e.g., characterized by a variation in a level of one or more of inflammatory cytokines, and optionally induced by surgery, chemical peritonitis, radiotherapy, and/or foreign body reaction); and a tissue repair and or remodeling condition.

A compound described herein can be administered to a subject in need thereof to potentially prevent, inhibit or treat one or more inflammation disorders. As used hereinafter, the terms “treating,” “treatment” and “therapeutic effect” can refer to reducing, inhibiting or stopping (preventing) an inflammation response (e.g., slowing or halting antibody production or amount of antibodies to a specific antigen), reducing the amount of inflamed tissue and alleviating, completely or in part, an inflammation condition. Inflammation disorders include, without limitation, allergy, asthma, autoimmune disorder, chronic inflammation, chronic prostatitis, glomerulonephritis, hypersensitivities, inflammatory bowel diseases, myopathy (e.g., in combination with systemic sclerosis, dermatomyositis, polymyositis, and/or inclusion body myositis), pelvic inflammatory disease, reperfusion injury, rheumatoid arthritis, transplant rejection, vasculitis, and leukocyte disorders (e.g., Chediak-Higashi syndrome, chronic granulomatous disease). Certain autoimmune disorders also are inflammation disorders (e.g., rheumatoid arthritis). In some embodiments, the inflammation disorder is selected from the group consisting of chronic inflammation, chronic prostatitis, glomerulonephritis, a hypersensitivity, myopathy, pelvic inflammatory disease, reperfusion injury, transplant rejection, vasculitis, and leukocyte disorder. In certain embodiments, an inflammation condition includes, but is not limited to, bronchiectasis, bronchiolitis, cystic fibrosis, acute lung injury, acute respiratory distress syndrome (ARDS), atherosclerosis, and septic shock (e.g., septicemia with multiple organ failure). In some embodiments, an inflammation disorder is not a condition selected from the group consisting of allergy, asthma, ARDS and autoimmune disorder. In certain embodiments, an inflammation disorder is not a condition selected from the group consisting of gastrointestinal tract inflammation, brain inflammation, skin inflammation and joint inflammation. In certain embodiments, the inflammation disorder is a neutrophil-mediated disorder. In some embodiments, an inflammatory condition also is a cell proliferation condition, such as, for example, inflammation conditions of the skin (e.g., eczema), discoid lupus erythematosus, lichen planus, lichen sclerosis, mycosis fungoides, photodermatoses, pityriasis rosea and psoriasis.

A compound described herein can be administered to a subject in need thereof to potentially treat one or more autoimmune disorders. In such treatments, the terms “treating,” “treatment” and “therapeutic effect” can refer to reducing, inhibiting or stopping an autoimmune response (e.g., slowing or halting antibody production or amount of antibodies to a specific antigen), reducing the amount of inflamed tissue and alleviating, completely or in part, an autoimmune condition. Autoimmune disorders, include, without limitation, autoimmune encephalomyelitis, colitis, autoimmune insulin dependent diabetes mellitus (IDDM), and Wegener granulomatosis and Takayasu arteritis. Models for testing compounds for such diseases include, without limitation, (a)(i) C5BL/6 induced by myelin oligodendrocyte glycoprotein (MOG) peptide, (ii) SJL mice PLP139-151, or 178-191 EAE, and (iii) adoptive transfer model of EAE induced by MOG or PLP peptides for autoimmune encephalomyelitis; (b) non-obese diabetes (NOD) mice for autoimmune IDDM; (c) dextran sulfate sodium (DSS)-induced colitis model and trinitrobenzene sulfonic acid (TNBS)-induced colitis model for colitis; and (d) systemic small vasculitis disorder as a model for Wegener granulomatosis and Takayasu arteritis. A compound described herein may be administered to a subject to potentially treat one or more of the following disorders, for example: Acute disseminated encephalomyelitis (ADEM); Addison's disease; alopecia greata; ankylosing spondylitis; antiphospholipid antibody syndrome (APS); autoimmune hemolytic anemia; autoimmune hepatitis; autoimmune inner ear disease; bullous pemphigoid; coeliac disease; Chagas disease; chronic obstructive pulmonary disease; Crohns disease (one of two types of idiopathic inflammatory bowel disease “IBD”); dermatomyositis; diabetes mellitus type 1; endometriosis; Goodpasture's syndrome; Graves' disease; Guillain-Barré syndrome (GBS); Hashimoto's disease; hidradenitis suppurativa; idiopathic thrombocytopenic purpura; interstitial cystitis; lupus erythematosus; mixed connective tissue disease; morphea; multiple sclerosis (MS); myasthenia gravis; narcolepsy; neuromyotonia; pemphigus vulgaris; pernicious anaemia; polymyositis; primary biliary cirrhosis; rheumatoid arthritis; schizophrenia; scleroderma; Sjögren's syndrome; temporal arteritis (also known as “giant cell arteritis”); ulcerative colitis (one of two types of idiopathic inflammatory bowel disease “IBD”); vasculitis; vitiligo; and Wegener's granulomatosis. In some embodiments, the autoimmune disorder is not a condition selected from the group consisting of Crohns disease (or Crohn's disease), rheumatoid arthritis, lupus and multiple sclerosis.

In some embodiments, a compound described herein is utilized in combination with the administration of one or more other therapies that include, but are not limited to, chemotherapies, radiation therapies, hormonal therapies, and/or biological therapies (e.g. immunotherapies). An agent that can be used in combination with a compound described herein can include, but is not limited to, a proteinaceous molecule, including, but not limited to, peptide, polypeptide, protein, including post-translationally modified protein, antibody and the like; small molecule (less than 1000 daltons); inorganic or organic compounds; nucleic acid molecule, including, but not limited to, double-stranded or single-stranded DNA, or double-stranded or single-stranded RNA, and triple helix nucleic acid molecules. An agent used in combination with a compound described herein can be derived from any known organism (including, but not limited to, animals, plants, bacteria, fungi, and protista, or viruses) or from a library of synthetic molecules. An agent that may be utilized in combination with a compound described herein includes a protein kinase inhibitor (e.g., a receptor protein kinase inhibitor) and an angiogenesis inhibitor.

The amount of the compound, or an active salt or derivative thereof, required for use in treatment will vary not only with the particular salt selected but also with the route of administration, the nature of the condition being treated and the age and condition of the patient and will be ultimately at the discretion of the attendant physician or clinician. In general a suitable dose sometimes is in the range of from about 0.5 to about 100 mg/kg, e.g., from about 10 to about 75 mg/kg of body weight per day, such as 3 to about 50 mg per kilogram body weight of the recipient per day, and often is in the range of 6 to 90 mg/kg/day, or about 15 to 60 mg/kg/day. A compound may be conveniently administered in unit dosage form, and for example, contain 5 to 1000 mg, or 10 to 750 mg, or 50 to 500 mg of active ingredient per unit dosage form. An active ingredient can be administered to achieve peak plasma concentrations of an active compound of from about 0.01 to about 100 pM, about 0.5 to about 75 pM, about 1 to 50 pM, or about 2 to about 30 pM. Such concentrations may be achieved, for example, by the intravenous injection of a 0.05 to 5% solution of an active ingredient, optionally in saline, or orally administered as a bolus containing about 1-100 mg of an active ingredient. Desirable blood levels may be maintained by continuous infusion to provide about 0.01-5.0 mg/kg/hr or by intermittent infusions containing about 0.4-15 mg/kg of the active ingredient(s). A desired dose may conveniently be presented in a single dose or as divided doses administered at appropriate intervals, for example, as two, three, four or more sub-doses per day. A sub-dose itself may be further divided, e.g., into a number of discrete loosely spaced administrations; such as multiple inhalations from an insufflator or by application of a plurality of drops into the eye.

### EXAMPLE

The examples set forth below illustrate, and do not limit, the technology.

### Example 1

### Examples of Compound Synthesis

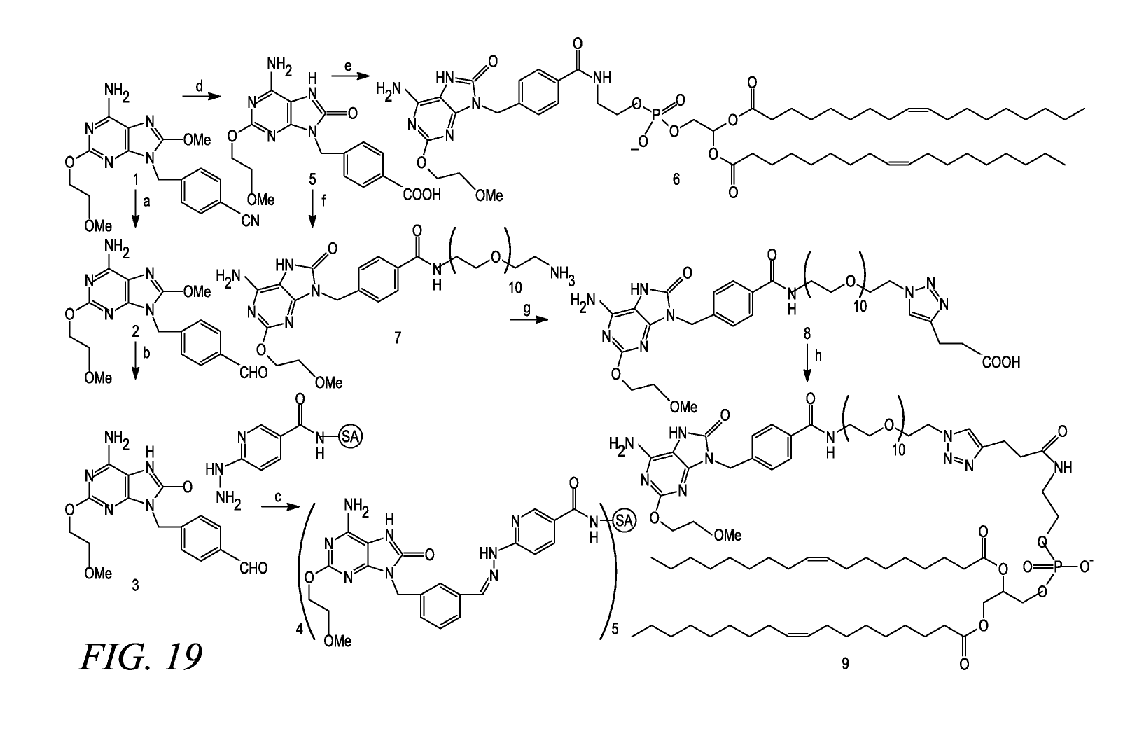

Compounds of Formula I, II and III can be synthesized using, for example, methods described in WO/2010/093436 published Aug. 19, 2010 (International Patent Application No. PCT/US2010/000369 filed Feb. 11, 2010), and examples are presented herein, and in FIG. 19. Certain examples of the compounds described herein, which are sometimes referred to herein as “TMX-X” compounds herein, are illustrated in Table 2 hereafter.

Chemical synthesis schemes described herein use numbers in parenthesis when referring to a compound in FIG. 19, and letters in parenthesis when referring to a reaction step (e.g., chemical(s) added and/or reaction conditions). For example, (a) refers to a reaction step that includes the addition of a reactant, which may result in the formation of compound (2), when combined and reacted with compound (1). The reaction conditions and compounds added for each reaction step are; (a) Lithium N,N′-methylethylenediaminoaluminum hydrides (Cha, J. et al., (2002) Selective conversion of aromatic nitriles to aldehydes by lithium N,N′-dimethylethylenediaminoaluminum hydride, Bull. Korean Chem. Soc. 23, 1697-1698), THF, 0° C.; (b) NaI, chlorotrimethylsilane, CH₃CN, r.t.; (c) PBS, r.t.; (d) NaOH:EtOH 1:1, reflux; (e) DOPE, HATU, triethylamine, DMF/DCM 1:1, r.t.; (f) O-(2-Aminoethyl)-O′-(2-azidoethyl)nonaethylene glycol, HATU, triethylamine, DMF, r.t.; (g) 4 pentynoic acid, sodium ascorbate, Cu (OAc)₂, t-BuOH/H₂O/THF 2:2:1, r.t.; and (h) DOPE, HATU, triethylamine, DMF/DCM 1:1, r.t.

Synthesis of 4-((6-amino-2-(2-methoxyethoxy)-8-oxo-7H-purin-9(8H)-yl)methyl)benzoic acid (see FIG. 19, compound 5). 20 mL of a 1:1 ethanol:water mixture was added to 0.10 g (0.28 mmol) of 4-((6-amino-8-methoxy-2-(2-methoxyethoxy)-9H-purin-9 yl)methyl)benzonitrile (see FIG. 19, compound 1), and the combination refluxed for 8 hours. The reaction mixture was allowed to cool and acidified to pH 2 with conc. HCl. The aqueous solution was further extracted with DCM (3×20 mL), dried over MgSO4 and evaporated in vacuo to yield a mixture of 8-oxo-9-benzoic acid (compound 5), 8-methoxy-9-benzoic acid and 8-oxo-9-ethyl benzoate. Once dried, the products were dissolved in CH₃CN (25 mL) and NaI (0.14 g, 0.96 mmol) was added (FIG. 19, reaction step (b)). To this solution was added 12 μL (0.96 mmol) of chlorotrimethylsilane, dropwise with stirring. The reaction mixture was heated at 40° C. for 4 hours then cooled, filtered and washed with water (20 mL) and then diethyl ether (20 mL) to obtain a white solid in 85% yield. Nuclear Magnetic Resonance (NMR) analysis was performed on the resultant product, with the following results, ¹H NMR (400 MHz, DMSO-d₆) δ (ppm): 10.33 (s, 1H), 7.89 (d, J=8 Hz, 2H), 7.37 (d, J=8 Hz, 2H), 6.65 (s, 2H), 4.92 (s, 2H), 4.24 (t, J=4 Hz, 2H), 3.56 (t, J=4 Hz, 2H), 3.25 (s, 3H). Retention time (Rt) on HPLC=14.3 min. ESI-MS (positive ion mode): calculated for C₁₆H₁₇N₆O₆ m/z [M+1] 360.34; found 360.24.

Synthesis of 2-(4-((6-amino-2-(2-methoxyethoxy)-8-oxo-7H-purin-9(8H)-yl)methyl)benzamido)ethyl 2,3-bis(oleoyloxy)propyl phosphate (see FIG. 19, compound 6). To a solution of 0.022 g (0.06 mmol) of compound 5 in 1 mL of anhydrous N,N-dimethylmethanamide (DMF) was added O-(7-azabenzotriazol-1-yl)-N,N,N′,N′-tetramethyluronium hexafluorophosphate (HATU) (0.026 g, 0.067 mmol) and anhydrous triethylamine (TEA) (17.0 μL, 0.12 mmol). A solution of 1,2-Dioleoyl-sn-glycero-3-phosphoethanolamine (0.05 g, 0.067 mmol) in anhydrous 1:1 dichloromethane (DCM):DMF (1 mL) was prepared and slowly added to the reaction mixture (FIG. 19 reaction step (e)). The reaction mixture was stirred at room temperature until completion and then evaporated in vacuo. The product was purified by flash chromatography using 15% methanol (MeOH) in DCM to give 0.038 g of white solid in 58% yield. NMR analysis was performed on the resultant product, with the following results, ¹H NMR (400 MHz, DMSO-d₆) δ (ppm): 9.7 (s, 1H), 7.87 (d, J=8.3 Hz, 2H), 7.32 (d, J=8.3 Hz, 2H), 6.61 (s, 2H), 5.30 (m, 4H), 5.05 (m, 1H), 4.88 (s, 2H), 4.26 (m, 4H), 4.06 (m, 1H), 3.77 (m, 4H), 3.57 (m, 2H), 3.35 (m, 2H), 3.26 (s, 3H), 2.23 (m, 4H), 1.95 (m, 8H), 1.46 (m, 4H), 1.22 (m, 40H), 0.83 (m, 6H). ESI-MS (negative ion mode): calculated for C₅₇H₉₂N₆O₁₂P m/z [M−1] 1083.35; found 1083.75.

Synthesis of 4-((6-amino-8-hydroxy-2-(2-methoxyethoxy)-9H-purin-9-yl)methyl)-N-(32-azido-3,6,9,12,15,18,21,24,27,30-decaoxadotriacontyl)benzamide (see FIG. 19, compound 7). To a solution of compound 5 (0.100 g, 0.278 mmol) in anhydrous DMF (5 mL) was added HATU (0.117 g, 0.306 mmol) and anhydrous TEA (77.014 μL, 0.556 mmol) (see FIG. 19, reaction step (f)). A solution of O-(2-aminoethyl)-O′-(2-azidoethyl)nonaethylene glycol (0.150 g, 0.306 mmol) in anhydrous DMF (1 mL) was prepared and slowly added to the reaction mixture. The reaction mixture was stirred at room temperature until completion and then evaporated in vacuo. The product was purified by flash chromatography using 5% MeOH in DCM to give 0.224 g of an opaque oil in 93% yield. Retention time on HPLC=12 min. NMR analysis was performed on the resultant product, with the following results, ¹H NMR (400 MHz, DMSO-d₆) δ (ppm): 10.01 (s, 1H), 8.45 (t, J=5.6 Hz, 1H), 7.78 (d, J=8.3 Hz, 2H), 7.35 (d, J=8.3 Hz, 2H), 6.49 (s, 2H), 4.90 (s, 2H), 4.25 (t, J=4 Hz, 2H), 3.57 (m, 4H), 3.5 (m, 36H), 3.4 (M, 6H), 3.26 (s, 3H). ESI-MS (positive ion mode): calculated for C₃₈H₆₁N₉O₁₄ m/z [M+1] 868.94; found 868.59.

Synthesis of 3-(1-(1-(4-((6-amino-8-hydroxy-2-(2-methoxyethoxy)-9H-purin-9-yl)methyl)phenyl)-1-oxo-5,8,11,14,17,20,23,26,29,32-decaoxa-2-azatetratriacontan-34-yl)-1H-1,2,3-triazol-4-yl)propanoic acid (see FIG. 19, compound 8). Compound 7 (0.218 g, 0.251 mmol) and 4-pentynoic acid (0.074 g, 0.753 mmol) were dissolved in 1:1 t-butanol:H2O (3 mL) (see FIG. 19, reaction step (g)). Sodium ascorbate (0.02 g, 100 mmol) and Cu(OAc)₂ (0.009 g, 50 mmol) in 1:1 t-butanol:H2O (1 mL) was slowly added to the reaction mixture and stirred at room temperature until compound 7 was fully reacted by TLC. The product was extracted with DCM (10 mL) and H2O (10 mL) and the organic layer was dried over MgSO₄ to give 0.230 g of an opaque oil in 95% yield. Retention time on HPLC=11.5 min. NMR analysis was performed on the resultant product, with the following results, ¹H NMR (400 MHz, DMSO-d₆) δ (ppm): 13.48 (s, 1H), 7.76 (d, J=8.29 Hz, 2H), 7.75 (s, 1H), 7.23 (d, J=8.29, 2H), 4.88 (s, 2H), 4.41 (t, J=5.12 Hz, 2H), 4.23 (t, J=4 Hz, 2H), 3.74 (t, J=5.12 Hz, 2H), 3.57 (t, J=4 Hz, 2H), 3.51 (m, 8H), 3.42 (m, 36H), 3.26 (s, 3H), 2.79 (t, J=7.56 Hz, 2H), 2.24 (t, J=7.56 Hz, 2H). ESI-MS (positive ion mode): calculated for C₄₃H₆₇N₉O₁₆ m/z [M+1] 966.04; found 966.67.

Synthesis of 2-(3-(1-(1-(4-((6-amino-8-hydroxy-2-(2-methoxyethoxy)-9H-purin-9-yl)methyl)phenyl)-1-oxo-5,8,11,14,17,20,23,26,29,32-decaoxa-2-azatetratriacontan-34-yl)-1H-1,2,3-triazole-4-yl)propanamido)ethyl 2,3-bis(oleoyloxy)propyl phosphate (see FIG. 19, compound 9). To a solution of compound 8 (96 mg, 0.1 mmol), HATU (42 mg, 0.11 mmol) in anhydrous DMF (1 mL) was added anhydrous TEA (2.7 μL, 0.2 mmol). A solution of DOPE (81.4 mg, 0.11 mmol) in 1:1 DCM:DMF (1 mL) was added dropwise to the reaction mixture and stirred at room temperature until completion (see FIG. 19, reaction step (h)). Upon completion the product was isolated by evaporation in vacuo followed by flash chromatography using 15% MeOH in DCM to give 155 mg of opaque oil in 92% yield. NMR analysis was performed on the resultant product, with the following results, ¹H NMR (400 MHz, DMSO-d₆) δ (ppm): 8.5 (s, 2H), 8.39 (s, 1H), 7.79 (m, 3H), 7.33 (d, J=6.23 Hz, 2H), 6.91 (s, 2H), 5.31 (m, 4H), 5.05 (m, 1H), 4.89 (s, 2H), 4.46 (m, 2H), 4.23 (m, 4H), 4.08 (t, J=8 Hz, 2H), 3.76 (m, 4H), 3.63 (t, J=8 Hz, 2H), 3.56 (t, J=8 Hz, 2H), 3.48 (m, 36H), 3.26 (m, 5H), 3.17 (m, 2H), 2.82 (t, J=8 Hz, 2H), 2.39 (t, J=8 Hz, 2H), 2.24 (m, 4H), 1.96 (m, 8H), 1.48 (m, 4H), 1.23 (m, 40H), 0.84 (m, 6H). ESI-MS (positive ion mode): calculated for C84H142N10O23P m/z [M+1] 1691.05; found 1692.82.

The synthesis of compound (4) from compound (1) yielded a consistent conjugation ratio of 5:1 UC1V150 to MSA protein (Wu, C. C. et al., “Immunotherapeutic activity of a conjugate of a Toll-like receptor 7 ligand”, Proc Natl Acad Sci USA 104, 3990-5 (2007)). Basic hydrolysis (FIG. 19, reaction step (d)) of the 9-benzylnitrile of compound (1) provided a versatile benzoic acid functional group (compound (5)) and allows for the assembly of conjugates (6), (8), and (9). The benzoic acid was coupled with DOPE by activation with HATU in the presence of TEA in anhydrous DMF (FIG. 19, reaction step (e)) to give compound 6 in 58% yield.

Due to the difficulty in dissolution of compound (6) in suitable solvents for testing, a PEG spacer was coupled to provide improved solubility. A readily available amine/azide bifunctional PEG was coupled to the benzoic acid by activation with HATU in the presence of TEA in anhydrous DMF (see FIG. 19, reaction step (f), which results in compound (7)). The formation of a 1,2,3-triazole through a copper(I)-catalyzed azide-alkyne cycloaddition with 4-pentynoic acid (FIG. 19, reaction step (g)) gave compound (8) in 95% yield. Finally, compound (9) was prepared by HATU activated amide formation with DOPE (FIG. 19, reaction step (h)) and compound (8).

TABLE 2

| Compound | | | Chain | | |
|---|---|---|---|---|---|
| # | Structure | MW | Length | Chain MW | m/Z |
| 1 | 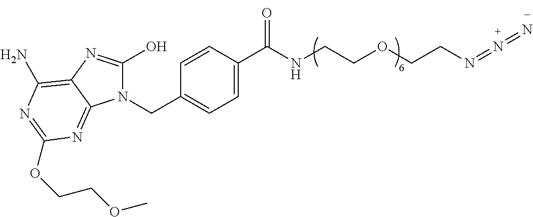 | 691.33 | 6 | 350.42 | 692.6 |
| 2 | 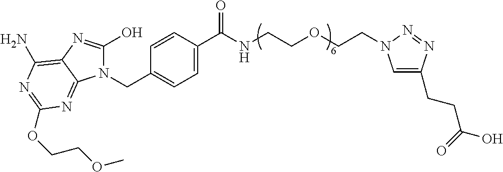 | 789.83 | 6 | 350.42 | 790.6 |
| 3 | 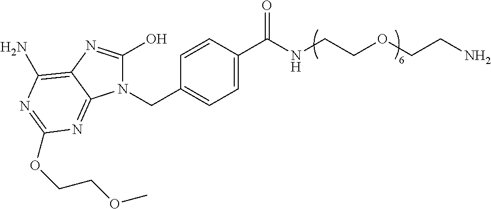 | 665.74 | 6 | 350.42 | 666.6 |
| 4 | 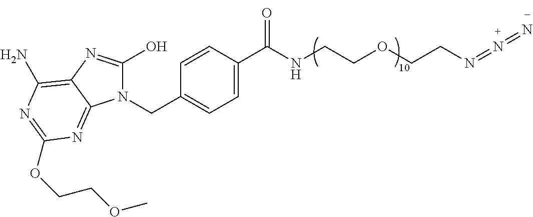 | 867.43 | 10 | 526.62 | 868.5 |
| 5 | 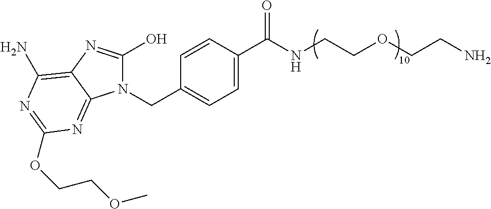 | 841.95 | 10 | 526.62 | 842.6 |
| 6 | 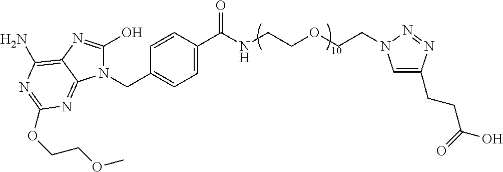 | 966.04 | 10 | 526.62 | 966.8 |
| 7 | 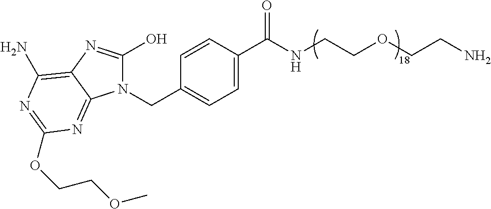 | 1238.42 | 18 | 897.1 | 1238.7 |
| 8 | 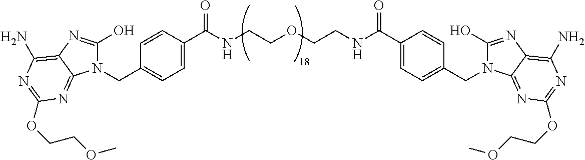 | 1579.74 | 18 | 897.1 | 1579.1 |
| 9 | 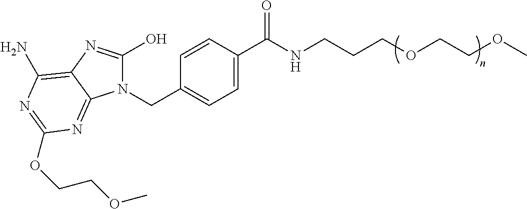 | 2525.13ₐ | 47ₐ | 2182 | b |
| 10 | 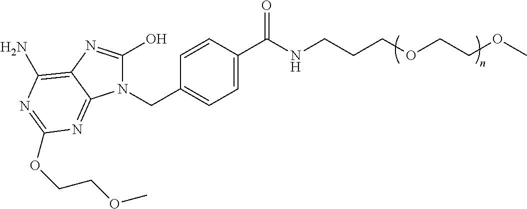 | 5572.13ₐ | 114ₐ | 5229 | b |
| 11 | 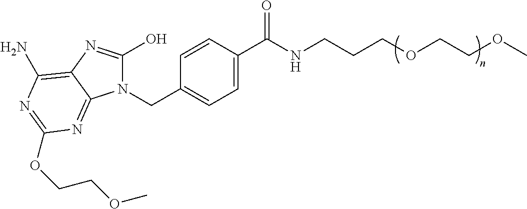 | 12609.13ₐ | 271ₐ | 12266 | b |
| 12 | 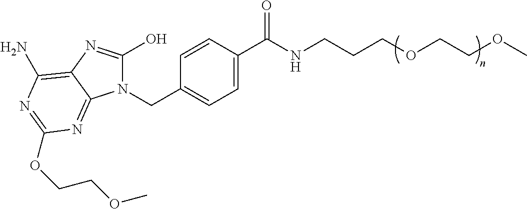 | 21701.13ₐ | 472ₐ | 21358 | b |

ₐApproximate molecular weight due to heterogeneity of PEG polymer.
b. Characterized by NMR only.

### Example 2

### Induction of Macrophage Tolerance

Mechanisms of Macrophage Tolerance

The general objective of these studies is to assess whether and to what extent certain compounds described herein, deemed to elicit tolerance, in fact activate an M2 program, with M1/M2 skewing, wherein M1 macrophage activation is reduced, and M2 macrophage activation is increased. These studies may pave the way to the identification of end points of the action of these compounds to be used as readouts.

Effects of Compound 2 of Table 2 on Macrophage Activation (In Vitro):

Compound 2 of Table 2 was tested as a selected test candidate of the compounds described herein. These compounds are TLR7-specific partial agonists that show anti-inflammatory properties in in vivo experiments.

Peritoneal macrophages (PEC) isolated from C57 black mice are used to test the tolerogenic effects of compound 2 of Table 2. The effects of this TLR7 ligand are analyzed in terms of gene expression (Real-Time PCR) of both pro-inflammatory (e.g. TNF-alpha, IL-1, IL-6) and anti-inflammatory (e.g. IL-10, TGF-beta) cytokines. Further, additional markers of M1 (e.g. iNOS, IL-12, IFN-beta, CXCL9, CXCL10) versus M2 (e.g. arginase I, Ym1, Fizz1, CCL17, CCL22, dectin 1, MGL) macrophage polarization is determined, in order to evaluate the polarizing activities of this compound. This analysis is instrumental to the understanding of mechanisms driving the tolerogenic action of compound 2 of Table 2, as an M2 polarized response is considered to be an alternative anti-inflammatory and “tolerogenic” macrophage program.

With respect to macrophage tolerance, cells are treated with two doses of compound 2 of Table 2 (1 microM and 10 microM) up to 20 hours and then re-challenged with agonists/ligands of different TLRs (TLR2, TLR4, TLR5, TLR9, TLR7, etc.), including the TLR7 unconjugated ligand 1V209 (free pharmacophore) at a concentration of 10 microM.

In addition, tolerance is tested also in response to primary cytokines IL-1-beta and TNFalpha. This approach clarifies whether the TLR7 partial agonist compound 2 of Table 2 promotes cross-tolerance of different members of the TLR family, as well as of the IL-1 and TNF receptor systems, which has been regularly reported.

TMX-mediated tolerance is promoted by the following scheme. TMX=Compound 2.

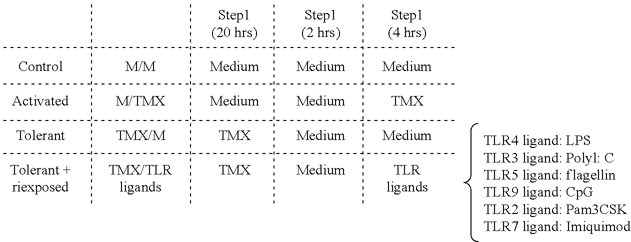

Based on the results of the gene expression analysis, performed by Real-Time PCR, selected gene products are validated at protein level, either by cytofluorimetry (surface molecules) or ELISA (secreted products).

As mentioned above, cells are treated with various TLR ligands (including TLR7 unconjugated ligands) after step 2, to determine both the specific- and the cross-tolerance activity of Compound 2.

Based on this analysis, and on an observation that LPS-tolerance in macrophages is associated with their M2 polarization (Porta C. et al PNAS 2009), activation of selected transcription factors involved in M1 (e.g. STAT1, STAT4) and M2 (e.g. STAT3, STAT6, p50 NF-kappaB) macrophage polarization, is analyzed.

The results provide a detailed characterization of the activation, tolerogenic and polarizing activity of compound 2 of Table 2 in macrophages, along with preliminary evidence on their therapeutic potential in chronic inflammation and autoimmune diseases.

Results

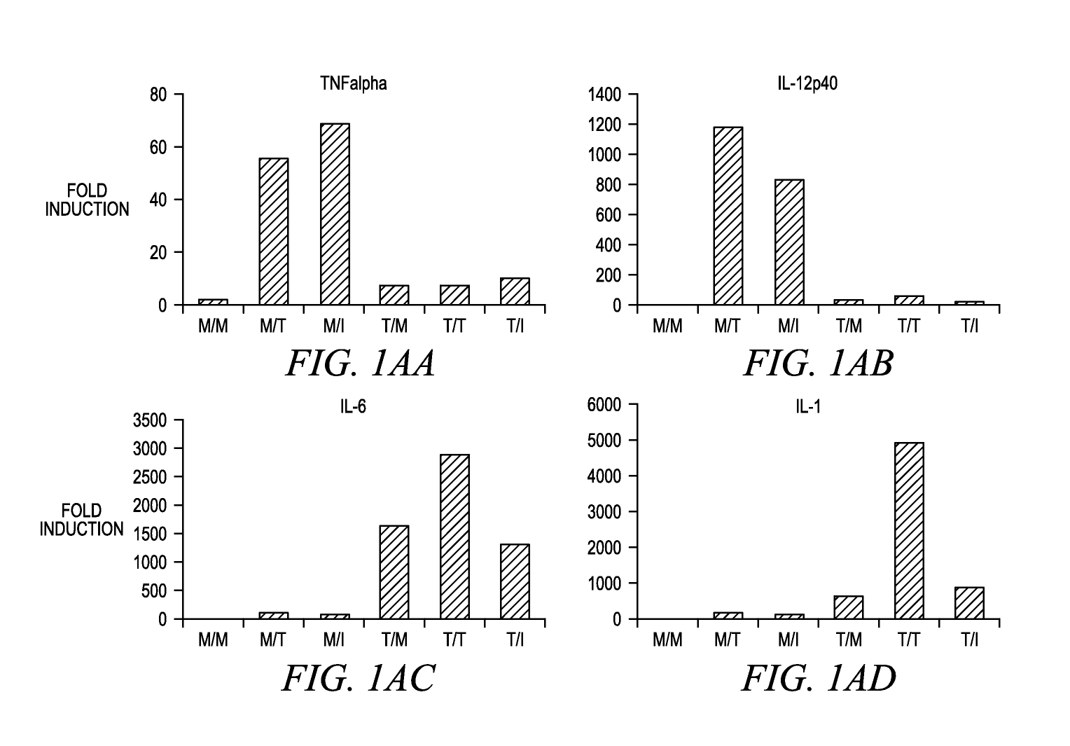

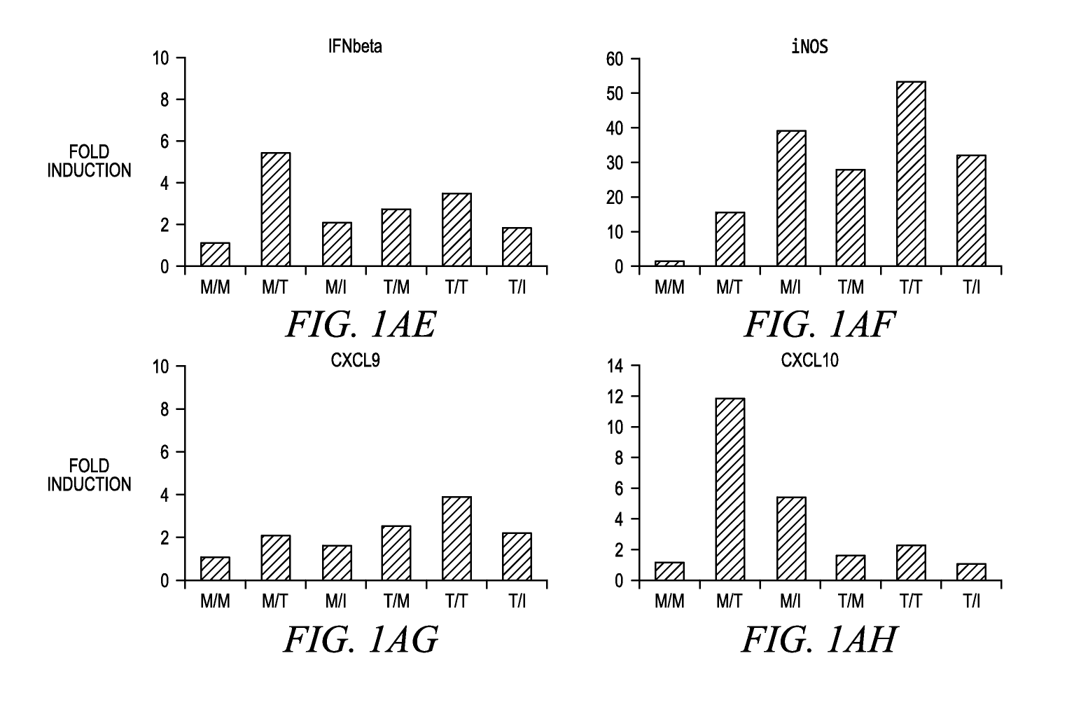

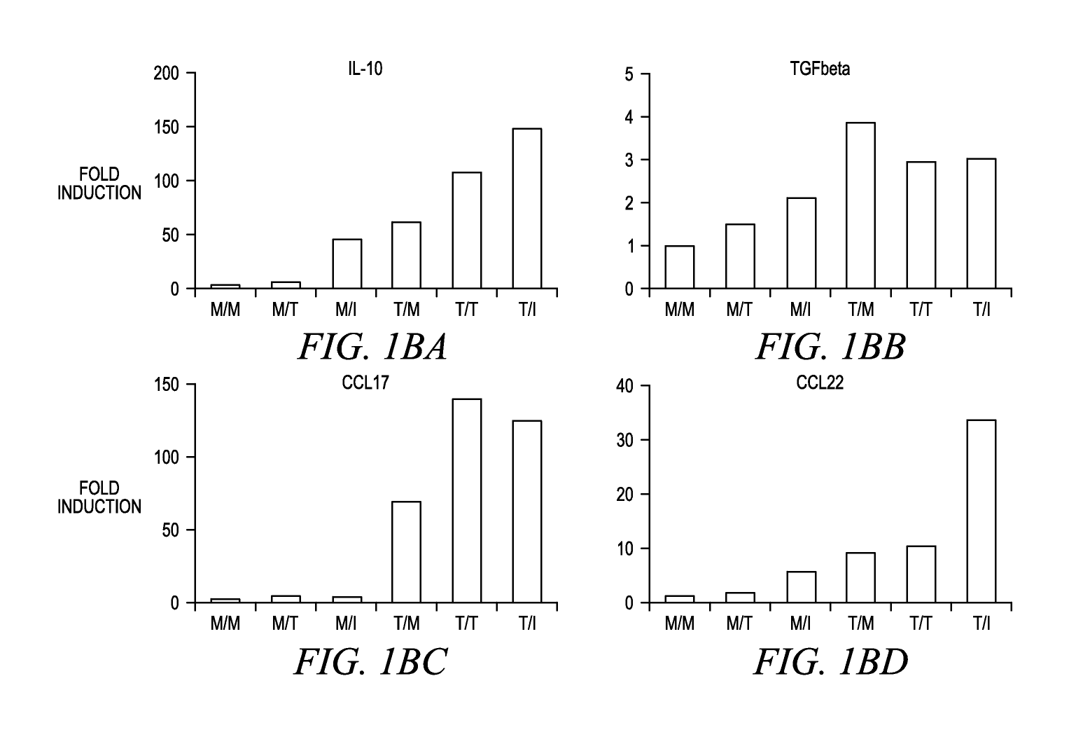

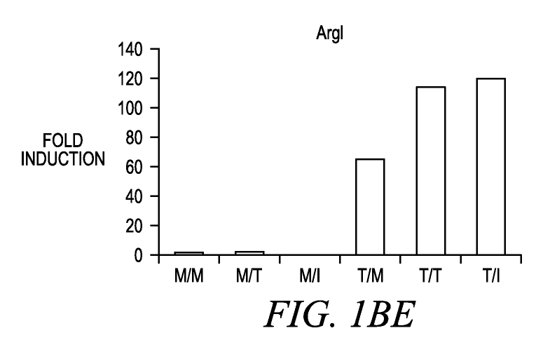

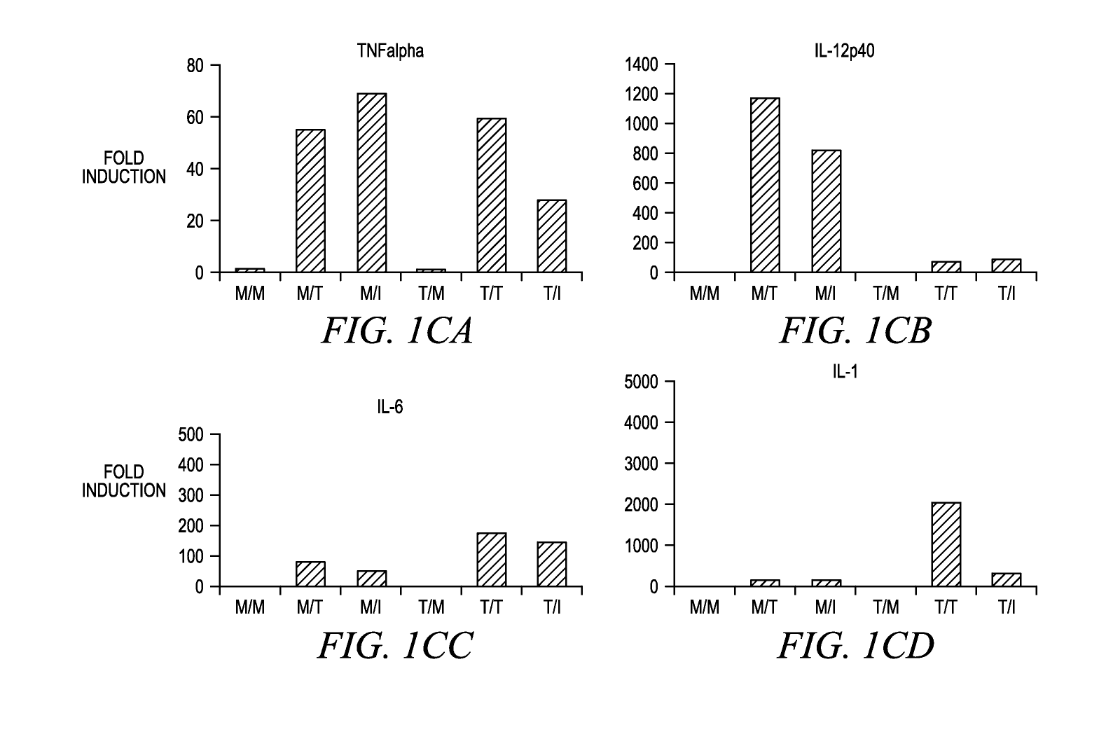

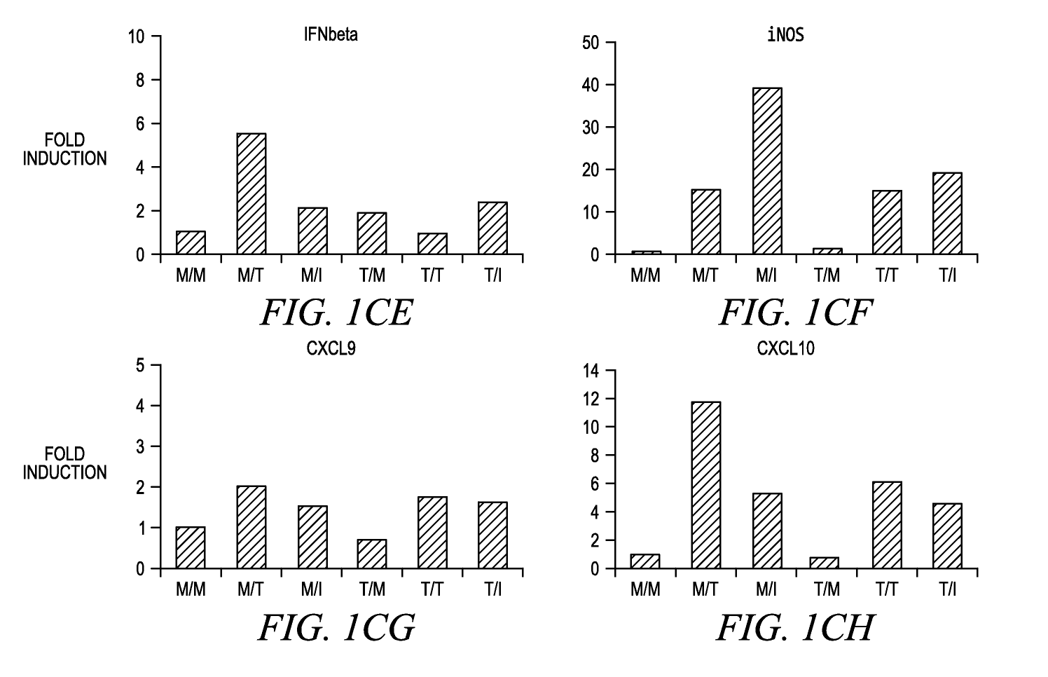

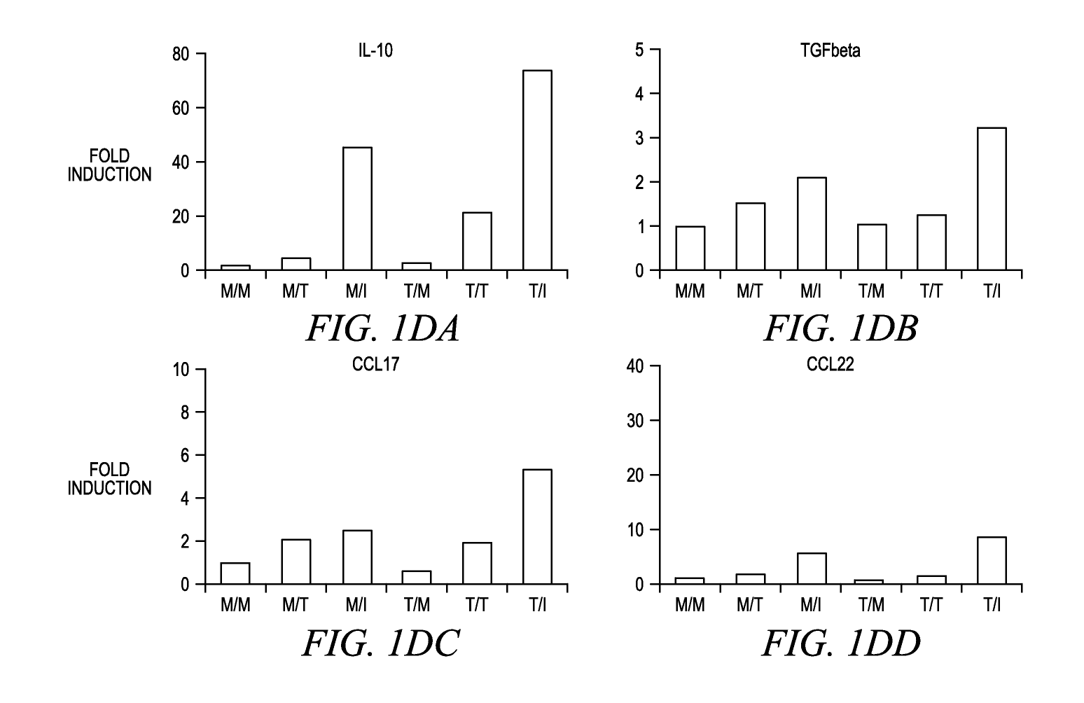

In a first run of experiments, the tolerogenic and polarizing ability of compound 2 of Table 2, which also is referred to hereafter as “TMX,” was evaluated. To induce tolerance, PEC were pre-treated for 20 hours with 10 μM or 1 μM TMX. Next, total RNA from control (M/M), TMX activated (M/T), 1V209 (free pharmacophore)-activated (M/I), tolerant (T/M) and tolerant PEC re-challenged with TMX (T/T) or with 1V209 (free pharmacophore) (T/I), were analyzed by RT PCR for the expression of representative M1 and M2 genes. The results showed that 10 μM of TMX induced a state of tolerance characterized by low M1 (TNF-alpha, IL-12, IFN-beta, CXCL10) gene expression along with high levels of selected M2 genes (IL-10, TGFβ, Arg I, CCL17, CCL22) (FIG. 1A, B). In contrast, pre-treatment with 1 μM of TMX, except for IL-12p40, was unable to induce tolerance. However, most of the M1 genes transcripts were still induced (FIG. 1C, D). Except for IL-10, 1 μM of TMX pre-treatment did not enhance M2 gene expression (FIG. 1D). No significant modulation of the M2 markers Ym1, Fizz1, MGL2 gene transcripts was observed. As compared to TMX, 1V209 agonist showed a similar potency in terms of induction of both M1 and M2 gene expression.

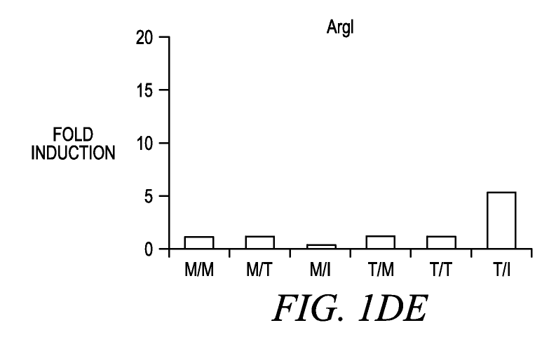

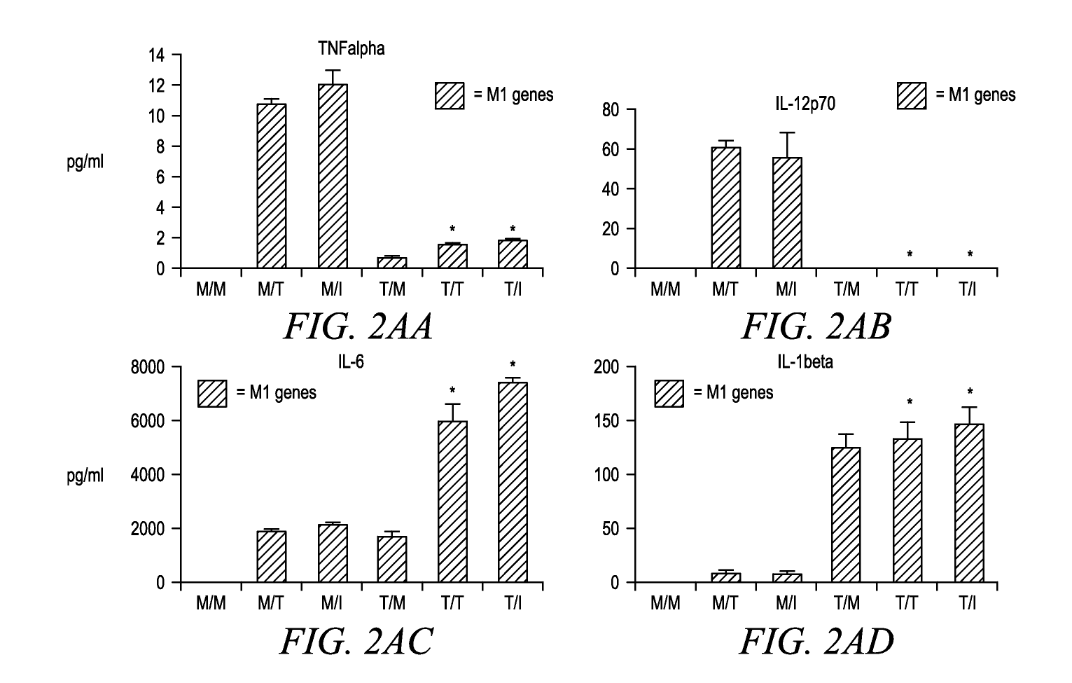

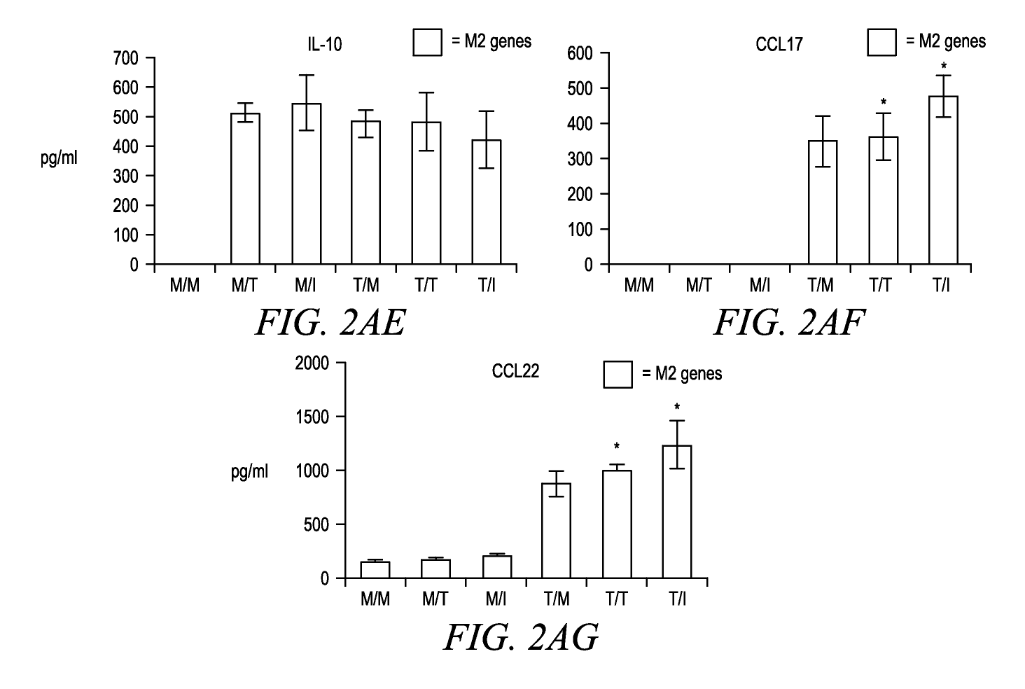

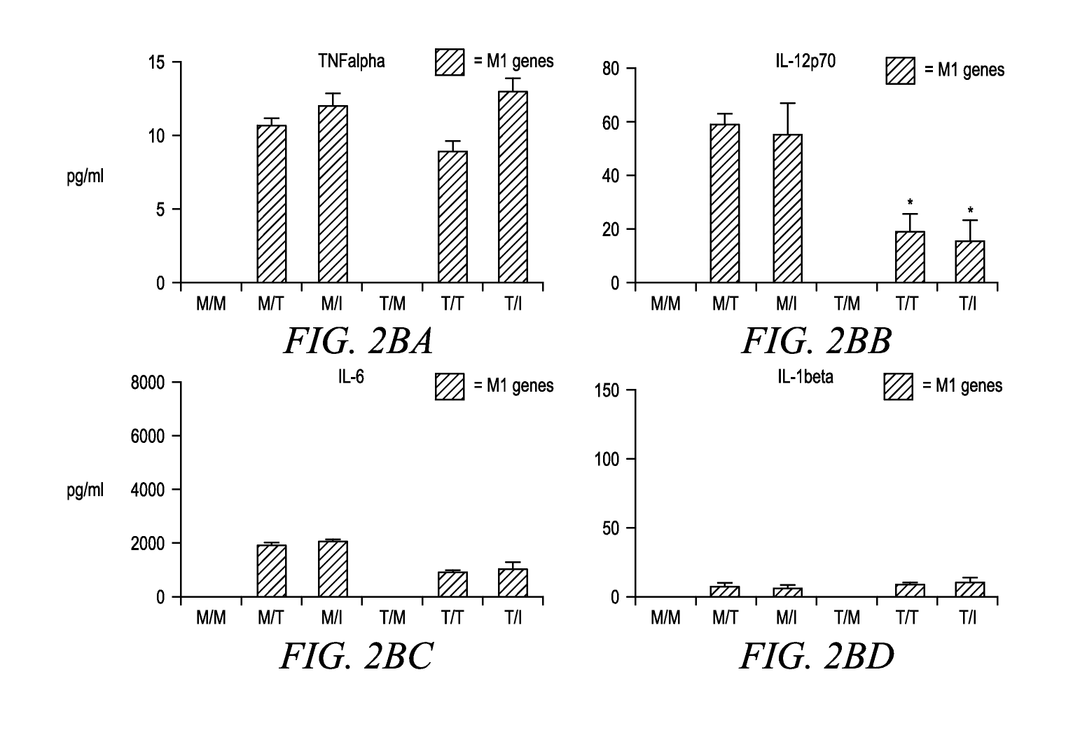

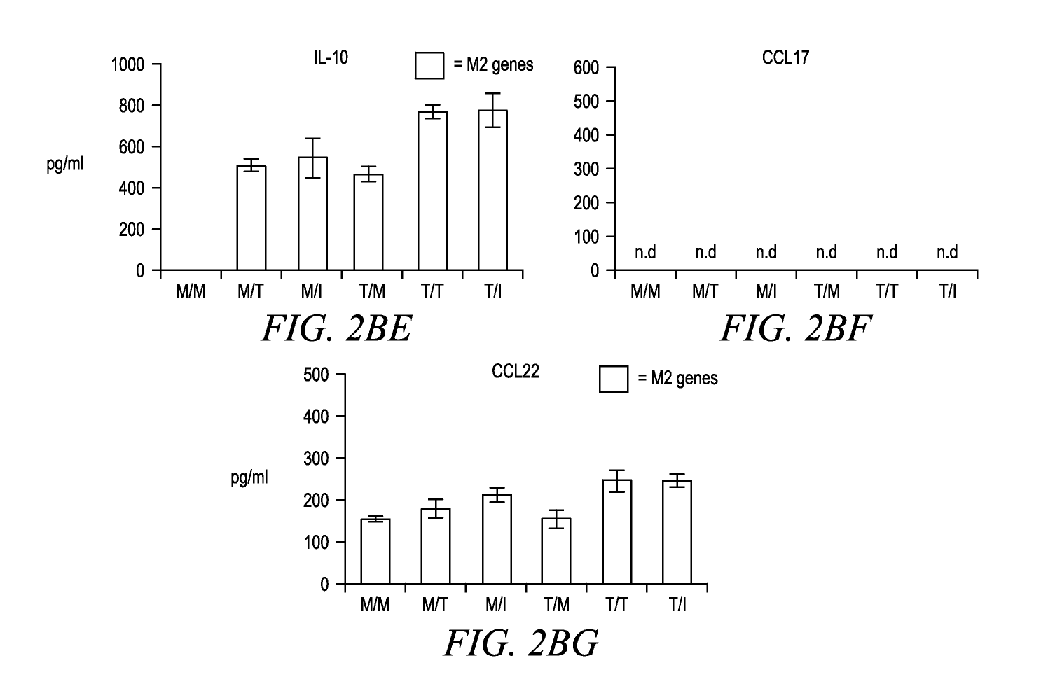

The secretion of selected M1 and M2 gene products by TMX-tolerant murine peritoneal macrophages (PEC) was evaluated. According to transcript levels, the results confirmed that only 10 microM TMX was able to induce a state of tolerance characterized by low M1 and high M2 cytokines production (FIG. 2). Strikingly, the increased levels of both IL-1p and IL-6 in the supernatant of TMX-tolerant macrophages was confirmed.

### Example 3

### Promotion of Cross-Tolerance by TMX Compounds

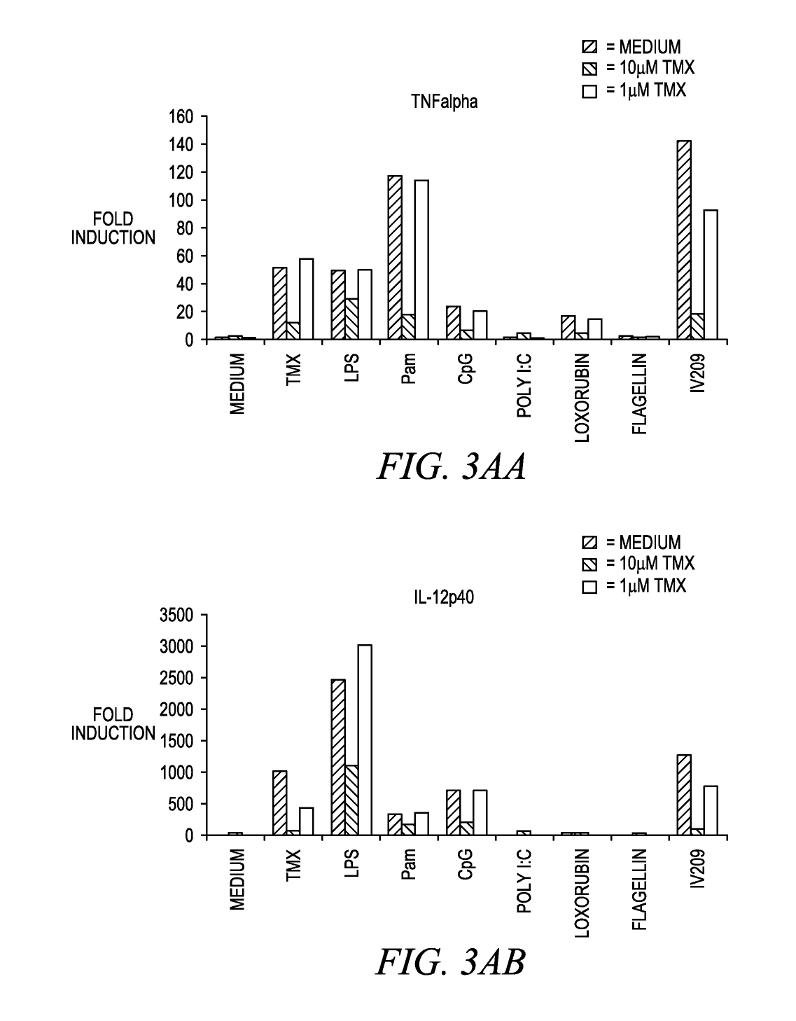

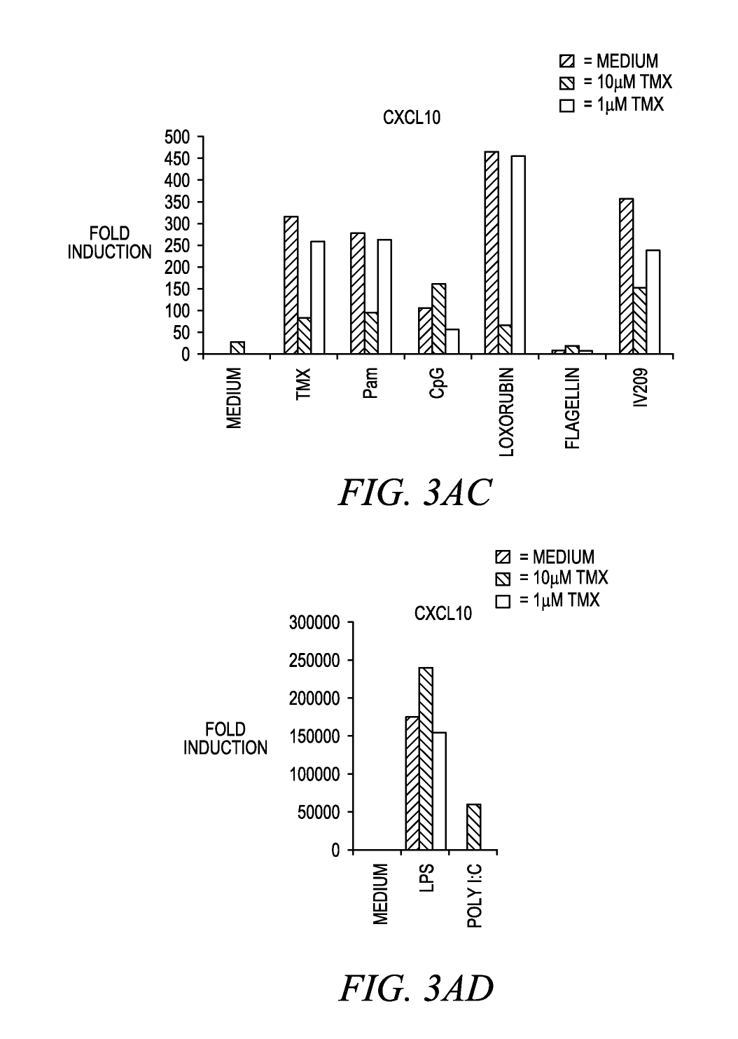

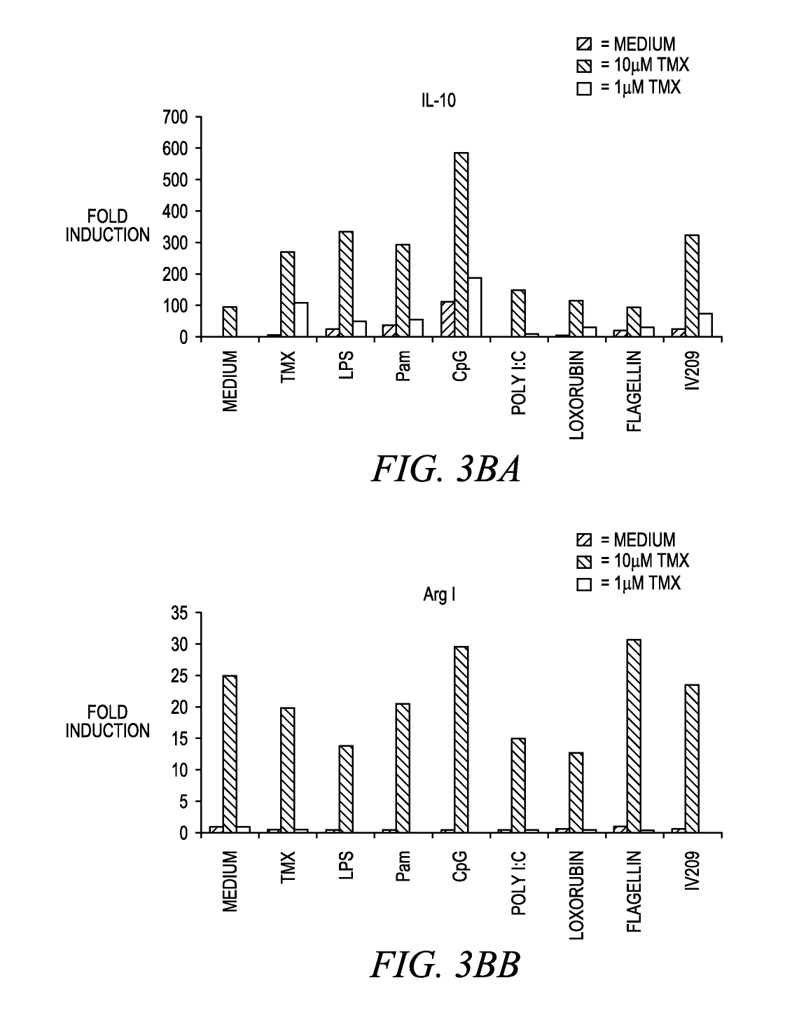

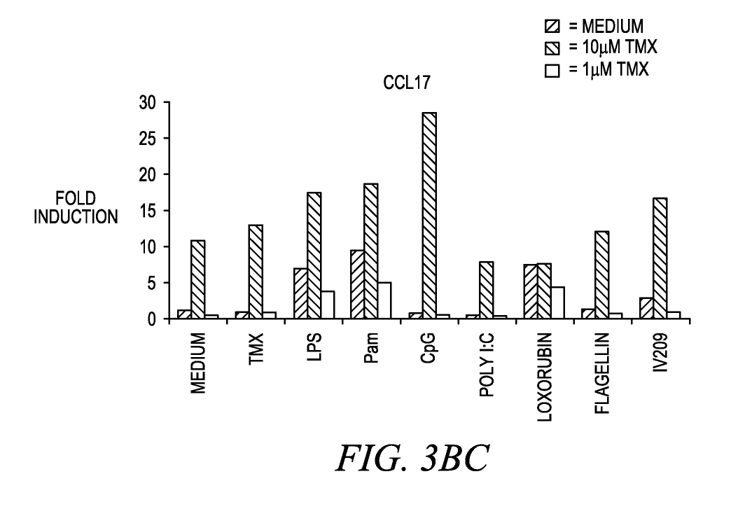

A second group of experiments was performed to evaluate the capacity of TMX to promote cross tolerance toward several TLR ligands and pro-inflammatory cytokines. PECs maintained in medium or pre-treated for 20 hours with 10 μM or 1 μM of TMX and re-challenged with several TLR agonists or pro-inflammatory cytokines were analyzed for the expression of selected M1 and M2 genes. The results show that only PECs tolerized by 20 hours pre-treatment with 10 μM TMX maintain the inability to up-regulate M1 genes expression in response to TLR ligands (FIG. 3A). In contrast different TLR agonists triggered the expression of M2 genes (FIG. 3B).

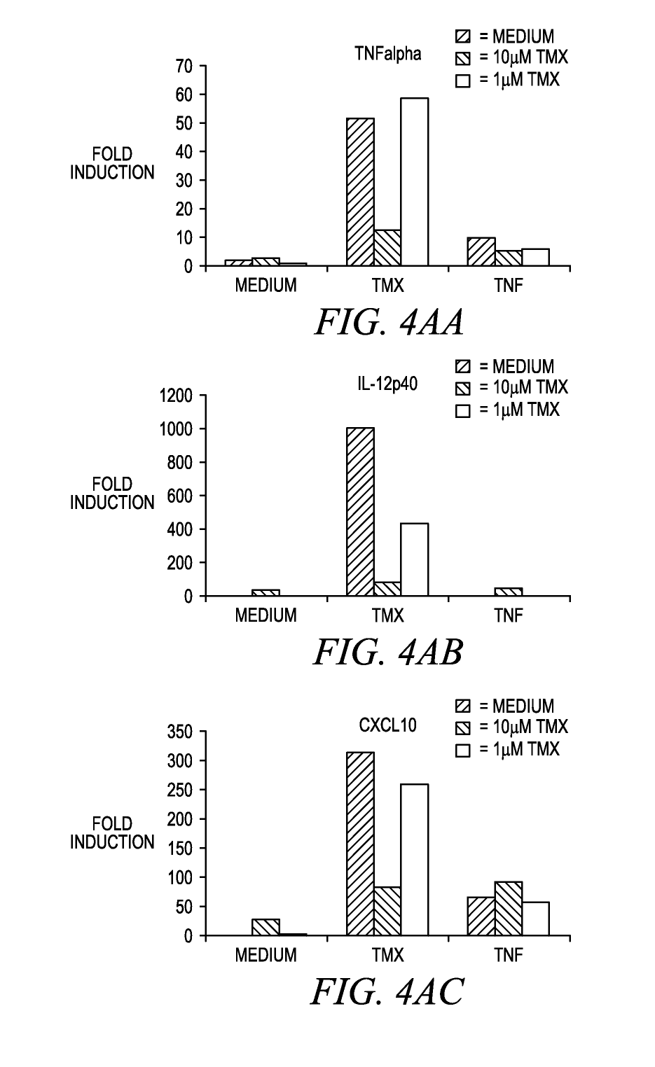

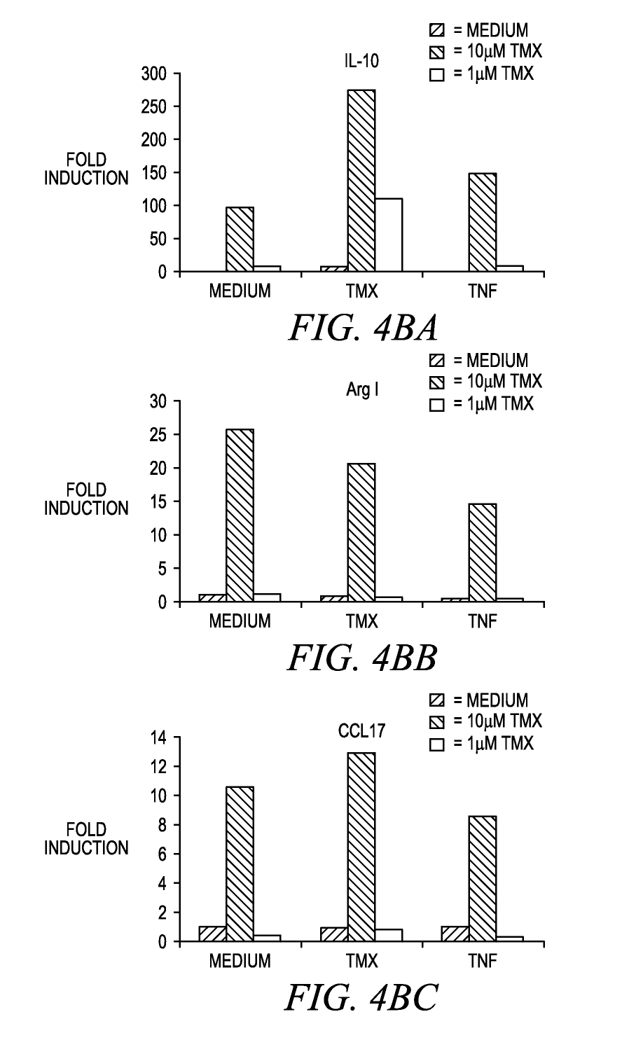

As compared to TLR ligands, induction of both M1 and M2 genes by TNF was weak (FIG. 4). Cross-tolerance in response to TMX 10 microM was observed. The only exception was the expression of CXCL10 following re-challenge with CpG, poly I:C, LPS. Cytokines (e.g. TNF) induced low levels of expression for the genes tested here. Among the M2 genes, only IL-10 seemed to be still induced following re-challenge of TMX-tolerant macrophages with TNF.

### Example 4

### Dose-Response Studies

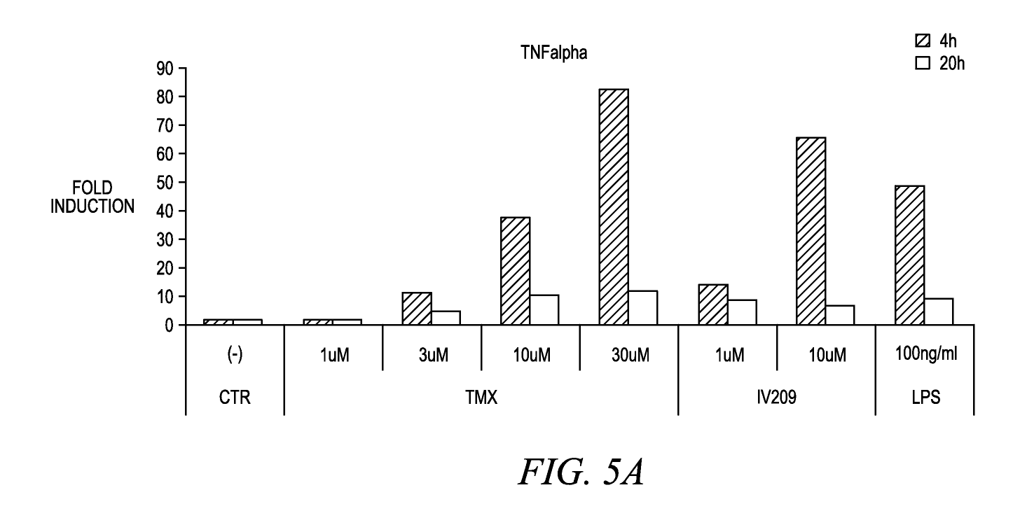

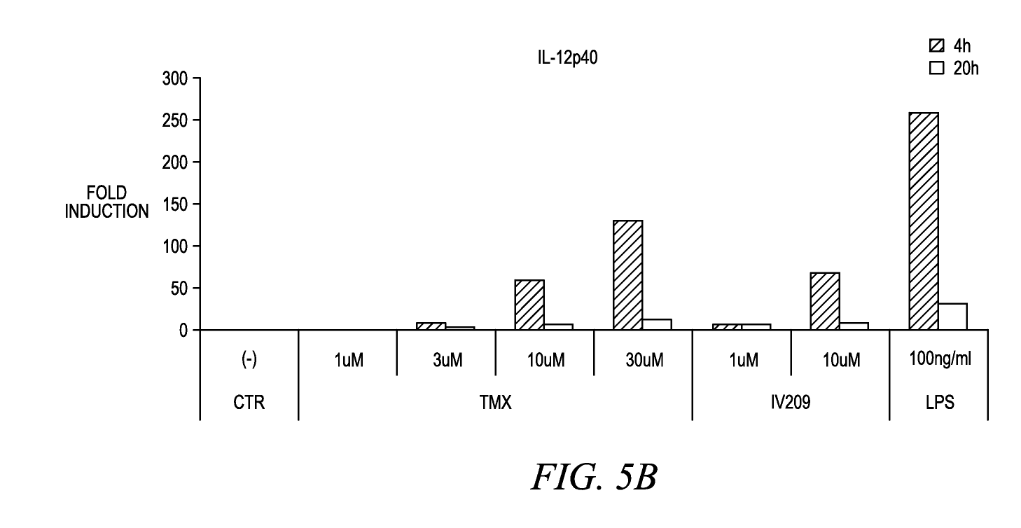

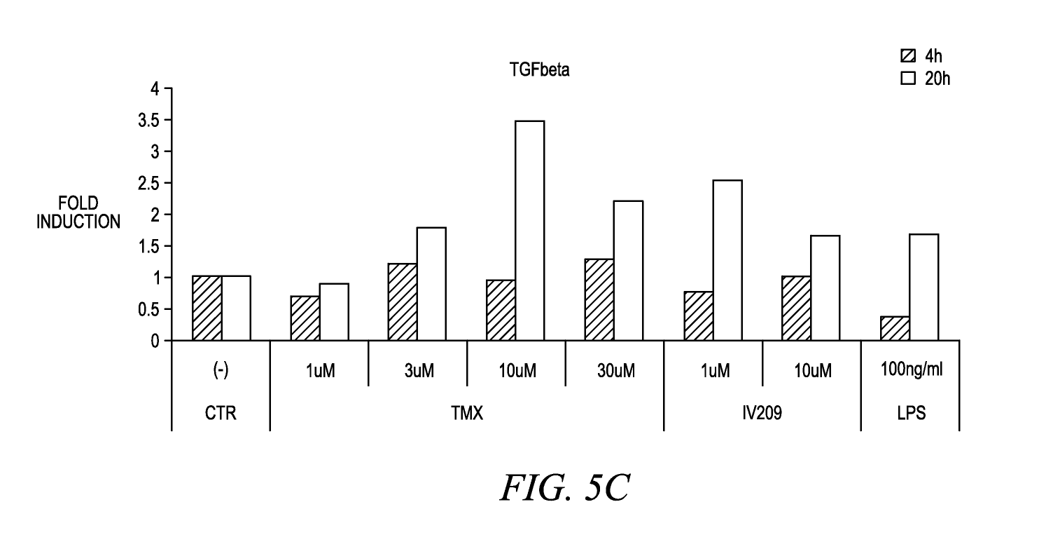

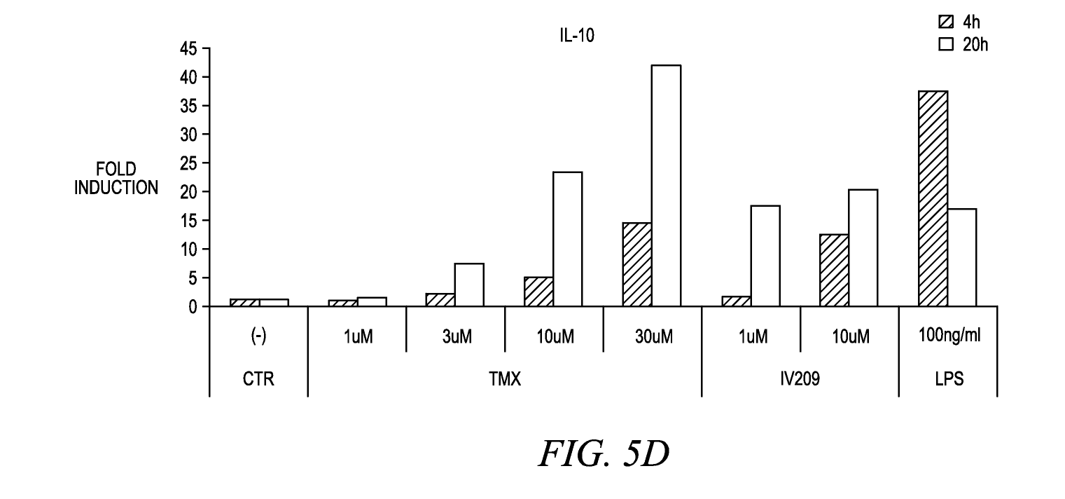

A third group of experiments was performed to investigate the dose-response effects of TMX, its potency as compared to classical inflammatory signals (e.g. LPS) and the activation of selected transcription factors playing an established role in tolerance, as well as in the induction of M1/M2 polarized inflammatory programs. PECs treated for 4 hours or 20 hours with 100 ng/mlof LPS or increased concentrations of TMX or 1V209 were analyzed by RT PCR for the expression of representative M1 (TNFα, IL-12p40) and M2 (IL-10, TGFβ) genes. The results showed a dose-dependent induction of both M1 and M2 gene expression (FIG. 5). In this experiment, as compared to 1V209, TMX is a lower inducer of M1 genes expression. In contrast, similar levels of M2 transcripts are triggered by both TLR7 agonists.

### Example 5

### NF-kappaB and Stats Activity Upon TMX-Mediated Activation and Tolerance

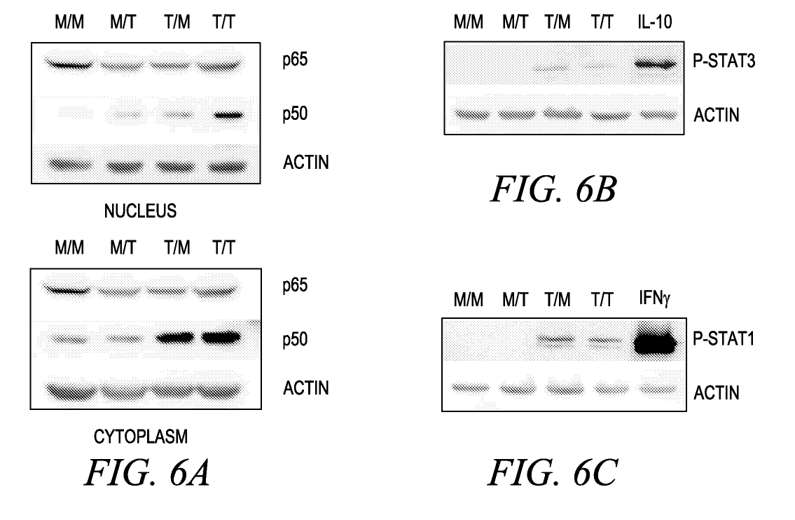

To investigate NF-κB and STATs activity upon TMX-mediated activation and tolerance, p65 and p50 NE-KB nuclear translocation and the levels of phospho-STATs proteins, respectively was analyzed. PEC treated 15 min with IFN-γ (500 U/mL) or with IL-10 (20 ng/ml) were used as positive controls for phospho-STAT1 and phospho-STAT3, respectively. In tolerance TMX 10 microM induces nuclear accumulation of p50 NF-kappaB and phosphorylation of STAT-3 (FIG. 6). In contrast, LPS induces nuclear accumulation of p50, without STAT-1 phosphorylation (FIG. 6). No detectable activation of STAT-6 in any condition was observed.

A dose-dependent induction of gene expression was observed. 30 microM was the most potent dose tested in these experiments, for the induction of both M1 and M2 genes. The capacity to promote tolerance against various M1 genes, along with the induction of both M2 and IFN-dependent genes (eg. iNOS, IFN-beta, CXCL10, CXCL9) and the activation of STAT1 is reminiscent of the TAM phenotype (Biswas, S. K., et al. (2006) Blood 1:2112-2122).

Materials and Methods:

Cell Culture and treatments. Peritoneal exudate cells (PEC) were harvested from mice injected with 1 ml of 3% (w/v) thioglycollate medium (Difco, Detroit, Mich.) 4 days prior to isolation. PECs were incubated in RPMI 1640 medium, containing 10% fetal bovine serum, 2 mM glutamine and 100 U/ml of penicillin-streptomycin. The concentration for the different treatments were as follows: LPS (100 ng/ml) (Lipopolysaccharide from Salmonella Abortus Equi S-form, Alexis), CpG (1 μg/ml), Pam3CSK (2 μg/ml), Poly I:C (10 μg/ml), Loxorubine (100 μM), flagellin (100 ng/ml) IL-1β (20 ng/ml), TNF (20 ng/ml), IL-10 (20 ng/ml) (Peprotech), IFNγ (200 U/ml) (Peprotech). To induce TMX tolerance, cells were incubated in the presence of TMX (10 μM or 1 μM) for 20 hrs, washed and maintained in RPMI medium for 2 hrs and then re-challenged with TMX (10 μM) for 4 hrs. To induce TMX-tolerization (T/M) cells were treated with TMX for 20 hrs, washed and then maintained in medium for additional 6 hours, without TMX re-challenge. To induce M1-activation, cells were incubated in medium for 20 hrs, washed, left in medium for 2 hrs and finally stimulated with TMX (10 μM) for 4 hrs. Control cells (M/M) were cultured in medium for the entire period of the experiment. For gene expression analysis, total RNA was extracted from TMX treated and untreated cells with trizol (Invitrogen), according to the manufacturer's instructions. To analyze cytokines and chemokines production supernatants were collected 24 hrs after the second challenge with TMX.

Real-Time PCR. Total RNA was purified as previously described. Briefly, reverse transcription from 1 μg of RNA was performed using cDNA Archive kit (Applied Biosystem, NJ, USA). Real time PCR was performed using Power Syber Green PCR Master Mix (Applied Biosystem, NJ, USA) and detected by 7900HT Fast Real-Time System (Applied Biosystem, NJ, USA). Data were processed using the SDS2.2.2 software (Applied Biosystem, NJ, USA). Results were normalized to the expression of the housekeeping gene β-actin and then expressed as folds of upregulation, with respect to the control cell population.

ELISA. Cell-free supernatants were tested by ELISA for the indicated cytokines/chemokines. Murine TNFα, IL-12p70, IL-6, IL-1β, IL-10, CCL17 and CCL22 ELISA kits were purchased from R&D Systems (Minneapolis, Minn.).

Western blot analysis. For the NF-κB proteins, nuclear and cytosolic extracts were analyzed by SDS/PAGE (10% acrylamide) as described. Immunoblotting was performed with rabbit anti-p50 (#1157), anti-p65 (#1226) antisera. For phospho-STATs analysis, protein extracts were prepared and processed as previously described. Briefly, cells were lysed with buffer containing 50 mM Tris-HCl, pH 8, 150 mM NaCl, 5 mM EGTA, 1.5 mM MgCl₂, 10% glycerol (v/v), 1% Triton X-100 (v/v), 10 mM Na₃VO₄, 10 mM Na₄P₂O₇, 50 mM NaF, 1 mM PMSF, 1× cocktail of protease inhibitors (Roche) for 30 min at 4° C. The lysates were centrifuged at 16,000 g at 4° C. for 30 min and the supernatants were run on a 7.5% (w/v) SDS-PAGE (35 μg protein/lane). Proteins were next transferred onto a nitrocellulose membrane (1 h at 100V) and immunoblotted for anti-phospho-STAT-1 or anti-phospho-STAT-3 (Cell Signaling Technologies Inc, MA) or anti-actin antibody (Santa Cruz Biotechnologies, CA). HRP conjugated anti-rabbit secondary antibody (Amersham) was used at 1:2000 dilution for 1 h at room temperature. Blots were visualized using Immun-Star™ HRP Chemiluminescent kit (Bio-Rad, USA) by ChemiDoc XRS instrument (Invitrogen). Then, optical density was determined by “Quantity One” software.

### REFERENCES

Porta C, Rimoldi M, Raes G, Brys L, Ghezzi P, Di Liberto D, Dieli F, Ghisletti S, Natoli G, De Baetselier P, Mantovani A, Sica A. Tolerance and M2 (alternative) macrophage polarization are related processes orchestrated by p50 NF-kappaB. PNAS, 2009; 106(35):14978-83
Medvedev A E, Sabroe I, Hasday J D, Vogel S N. Tolerance to microbial TLR ligands: molecular mechanisms and relevance to disease. J Endotoxin Res. 2006; 12(3):133-50.
Mantovani A, Sica A, Locati M. Macrophage polarization comes of age. Immunity. 2005; 23:344-346.

### Example 6

### Use of Compounds for Alleviating and Treating Diseases and Conditions

Certain partial agonist compounds described herein seem to mimic the effect of repeated exposure to low doses of TLR agonists in inducing “tolerance” and thus reducing inflammation in vivo (Hayashi T. et al. (2009) PNAS 106:2764-9). These compounds show promising properties as anti-inflammatory compounds in various experimental animal models of autoimmune and inflammatory diseases, as well as of fibrosis. Due to their weak (partial agonistic) activity, these compounds may not cause side effects up to very high doses in animal models and, therefore, may be safely applied systemically.

Certain compounds described herein are composed of a TLR7-specific small molecule ligand conjugated with short, single polyethylene glycol (PEG) chains. The compounds are TLR7 “partial agonists” with anti-inflammatory properties in vivo.

Potential indications for the development of certain compounds described herein are inflammatory diseases in general, fibrosis, autoimmune conditions, and tissue repair.

### Example 7

### Drug Substance and Drug Products

Drug Substance

Certain compounds described herein are purine-like molecules conjugated with PEG chains of different lengths (6, 10, 18, 47 carbon atoms) with different terminal functional groups. The synthesis process consists of 7 steps for the preparation of the pharmacophore (1V209, free pharmacophore linked to a carboxyl group) plus an additional 2 steps for the conjugation. The yield after conjugation is, for example, around 50% with a purity of about 96% (HPLC area).

Solubility Profile in Organic and Inorganic Solvents of the API

The solubility of Compound 6, Compound 2, and Compound 3 was measured in the following conditions: distilled water, PBS buffer pH 7.4, HCl 0.1 M, ethanol, ethylacetate, acetic acid, methanol, DMSO, acetone, glycerol, propylene glycol, and polyethylenglycol 200.

The solubility for all three compounds was higher than 2 mg/ml in distilled water, PBS pH 7.4, HCl 0.1 M, DMSO, acetic acid and about 2 mg/ml (or slightly less) in methanol and ethanol. The solubility was between 0.5 to 1.6 mg/ml for the remaining solvents, except for ethylacetate, in which the solubility was significantly reduced (<0.3 mg/ml).

Stability of the API

Compound 2 powder underwent preliminary stability testing at 25° C. for 6 months and 40° C. for three months. In both conditions Compound 2 showed excellent stability properties with no degradation or impurity profile changes. Aqueous solutions of Compound 2 (2 mg/ml) are stable up to 4 weeks at 37° C.

Formulation and Drug Product

Compound 2 was formulated as an aqueous solution for ocular instillation and topical gel for skin application. In both 10% Compound 2 formulations, a small quantity of a thickening agent, cellulose (hydroxypropyl cellulose) was used. The solution for ocular instillation can be sterilized by filtration thought 0.1 μm sterilizing filters.

Physical/Metabolic Stability, PK and Toxicity Studies

Certain compounds described herein show good physical and metabolic stability, with low metabolism in mouse fresh hepatocytes. Intravenous acute toxicity experiments in mice showed that Compound 2 did not produce toxic clinical signs in treated animals up to 1000 mg/kg i.v. (intravenous) PK analysis after i.v. administration in mice indicated that Compound 6 and Compound 2 showed lower Cmax and AUC, higher clearance and longer half life in comparison with Compound 3. All three compounds showed very low BBB penetration in mice. Permeability in intestinal membranes (Caco-2 in vitro model) appears to be low/medium for all three compounds.

### Example 8

### Effects of Compound 2 on Experimental Lung Fibrosis

The efficacy of Compound 2 on mouse models of idiopathic lung fibrosis was studied as follows. Lung fibrosis was induced in 6-8 week old female C57BU6 mice. 0.8 U/kg of bleomycin (Hospira, Inc., Lake Forest, Ill.) was administered by intratracheal (i.t.) instillation on day 0. Bleomycin treated C57BU6 mice were divided into four groups (n 7 per group). Groups 2 and 4 were subcutaneously treated with Compound 2 at daily dose of 500 nmol/animal. Groups 1 and 3 were treated with saline control. Groups 1 and 2 were sacrificed on day 7 and groups 3 and 4 were sacrificed on day 21.

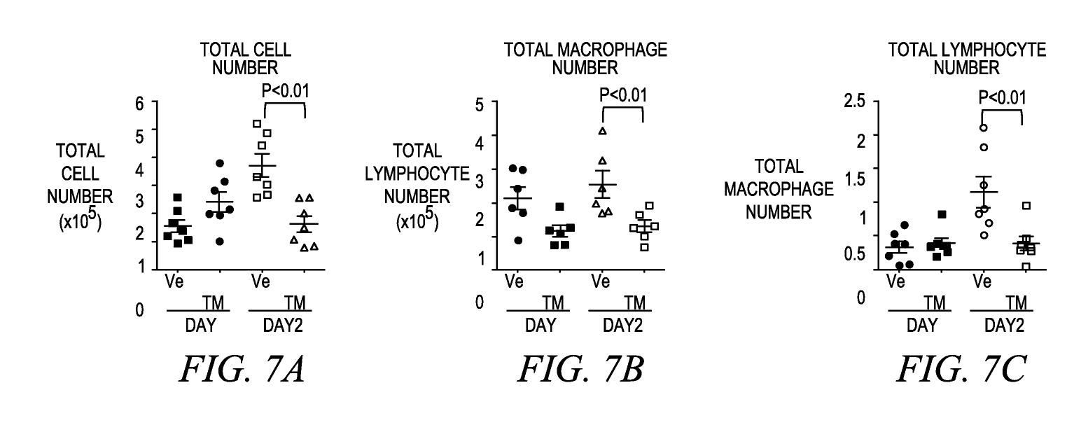

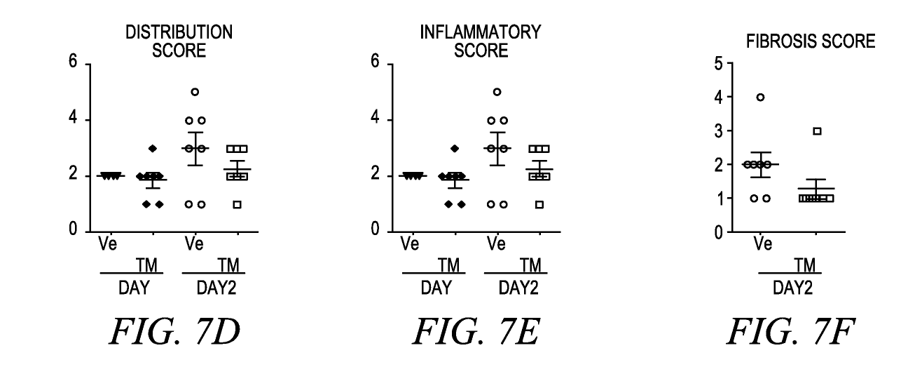

Measurements: Lung inflammation and fibrosis were evaluated by the following parameters: 1) cell infiltration to bronchial alveolar lavage (BAL); BAL were collected at the time of sacrifice and cell number was counted by hematocytometer. The infiltrated cells were differentiated by Wright-Giemsa staining. 2) BAL cytokines (IL-6, and IL-1b) by ELISA). 3) Lung histology by H&E staining and Sirius red staining (fibrosis). Histological samples were evaluated blindly. Results are shown in FIG. 7 and in Table 3.

TABLE 3

| | | | Induction (Day 0) | | | | | |
|---|---|---|---|---|---|---|---|---|
| Grp # | Group | n | TX | Route | Volume | TX | Route | Volume |
| | | | | | | Treatment (Day 0-6) | | |
| 1 | C57Bl/6 | 7 | 0.8 U/kg | Intratracheal | 50 ul | Saline | SC | 100 ul |
| | | | Bleomycin | | | | | |
| 2 | C57Bl/6 | 7 | 0.8 U/kg | Intratracheal | 50 ul | Cpd 2 | SC | 100 ul |
| | | | Bleomycin | | | 500 nmol | | |
| | | | | | | Treatment (Day 0-20) | | |
| 3 | C57Bl/6 | 7 | 0.8 U/kg | Intratracheal | 50 ul | Saline | SC | 100 ul |
| | | | Bleomycin | | | | | |
| 4 | C57Bl/6 | 7 | 0.8 U/kg | Intratracheal | 50 ul | Cpd 2 | SC | 100 ul |
| | | | Bleomycin | | | 500 nmol | | |

### Example 9

### Collagen-Induced Arthritis in Rats: Dose-Dependent Reduction of Paw Thickness Upon Treatment with Compound 2

### Summary

The effect of different concentrations of Compound 2 on arthritis development in the rat collagen-induced arthritis (CIA) model was evaluated. In this model, arthritis is induced by immunization with bovine type II collagen and signs of arthritis start to develop approximately 10 days after immunization.

The aim of this study was to determine the effect of different concentrations of Compound 2, a partial TLR-7 agonist, on the development of collagen-induced arthritis (CIA) in the rat.

Collagen-induced arthritis was successfully induced in this study as judged by the 100% disease incidence in the vehicle group in conjunction with increasing total clinical score over time, reaching a maximal value of 8.1 a.u. Therapeutic treatment with dexamethasone (intraperitoneal) resulted in a significant suppression of the clinical symptoms of arthritis, reaching a reduction in cumulative arthritis score of approximately 97%.

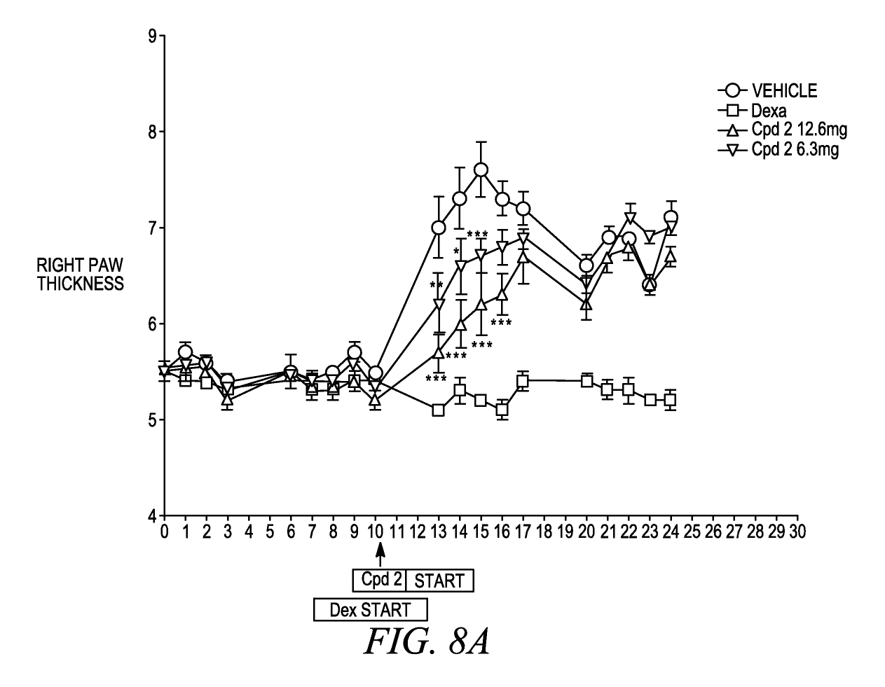

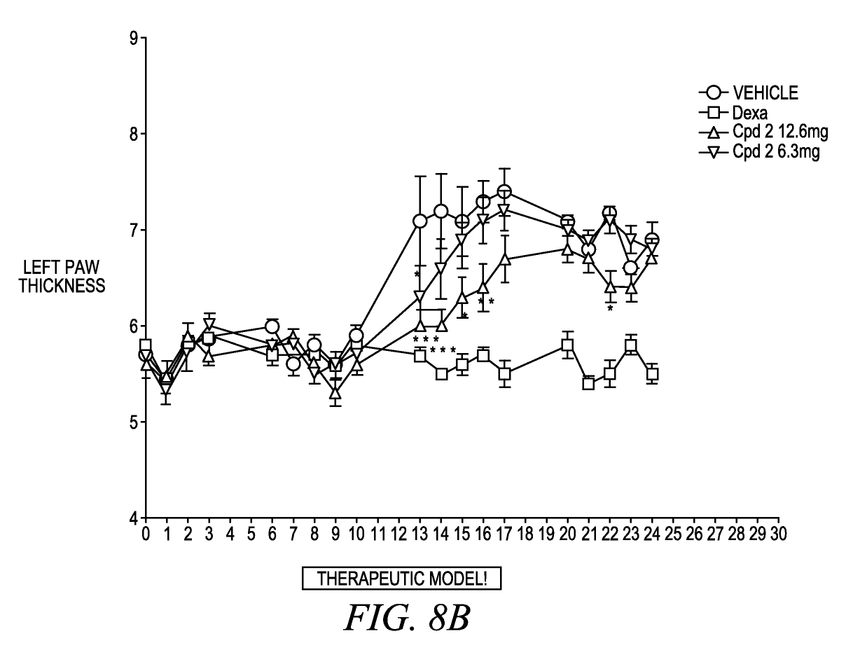

Therapeutic treatment with Compound 2 at a dose level of 12.6, 6.3, 3.2 and 1.6 mg/kg was applied in this study. The severity of arthritis was assessed by a semi-quantitative scoring of all four paws, by measuring the increase in hind paw thickness by a laser caliper and histopathological analysis on the left ankle joints. Dexamethasone treatment at a dose level of 0.5 mg/kg was included as a positive control to show that the model is sensitive to treatment. Compound 2 and dexamethasone treatment started from the day the first animals showed signs of arthritis (day 10). The development of arthritis was reflected by swelling of the hind paws. Swelling of the hind paws started around day 10, reaching a maximum on days 15-17. The mean cumulative hind paw swelling reached a value of 13.6±2.6 and 14.8±2.6 a.u. (mean±SD) for the left and right hind paw, respectively. Thickness of the hind paws was measured employing a laser scan micrometer. The paw thickness was measured 5 times per week. Each point represents the group mean (n=8). Results are shown in FIG. 8

Treatment with Compound 2 at a dose level of 6.3, 3.2 or 1.6 mg/kg did not significantly reduce arthritis development compared to the vehicle group as judged from the different clinical arthritis and histopathological parameters. The highest dose group (12.6 mg/kg), on the other hand, showed significantly reduced swelling of the hind paws, although this could not be substantiated by the cumulative arthritis score. However, in the initial phase of the disease the total clinical arthritis score was suppressed at this dose level of Compound 2, reaching significant differences on day 13 and 16 compared to the vehicle group. This suppression disappeared in a later stage of disease and clinical arthritis scores even statistically exceeded that from the vehicle group from day 22 onward, which may explain why no effect on cumulative arthritis score was observed compared the vehicle group. In addition, histopathological analysis revealed also a significantly diminished loss of proteoglycans in the 12.6 mg/kg Compound 2 group. Altogether, the data suggest a limited effect of the highest Compound 2 dose on the development of arthritis in this rat CIA model.

### Experimental Design

Collagen-induced arthritis was induced in 9 weeks old female Lewis rats using a two-steps immunization protocol. On day 0, rats were immunized by intradermal injection of approximately 1 ml of 1 mg/ml bovine type II collagen (Chondrex) emulsified in Incomplete Freund's adjuvant (Difco Laboratories) at several sites at the back. Arthritis development was accelerated by an intradermal booster immunization in the back and tail-base with 100 □g bovine type II collagen (Chondrex) in IFA (Difco Laboratories) at day 7. For immunization, rats were anesthetized by inhalation of 3-4% isoflurane in a mixture of oxygen and nitrous oxide.

At day 10 (day that first animals showed signs of arthritis, onward, the rats were injected once daily. The study groups included intraperitoneal injections as follows, for each group, n=8. (1) Vehicle (PBS); (2) Dexamethasone (dexamethasone 21-acetate, Sigma), 0.5 mg/kg; (3) Compound 2, 12.6 mg/kg; (4) Compound 2, 6.3 mg/kg; (5) Compound 2, 3.2 mg/kg; (6) Compound 2, 1.6 mg/kg. Compound 2 was prepared in PBS.

### Clinical Arthritis Score

Rats were evaluated 6 times per week (once during weekends) for arthritis severity using a macroscopic scoring system of 0-4 for each paw as detailed below:

0=no signs of arthritis
0.5=unloading of the paw and/or light redness of ankle joint
1=redness and mild swelling of the ankle joint
2=redness and swelling of paw
3=severe redness and swelling of entire paw including digits
4=maximally swollen paw, often involvement of multiple joints and extending toward knee joint.

The total clinical score of an individual rat is defined as the sum of the clinical scores of all four paws for each day. At the end of the study, the cumulative arthritis score was calculated for each rat. This cumulative arthritis score is defined as the sum of the total clinical scores obtained from day 0 till day 24.

### Day of Disease Onset

The day of disease is defined as the first day of three consecutive days on which a total clinical arthritis score of more than 0 was observed. If rats did not develop disease during the experimental period, the day of disease onset was arbitrarily set to day 24.

### Hind Paw Swelling

The swelling of the hind paws was measured during weekdays with a laser scan micrometer (Mitutoyo, LSM-503S/6200). At the end of the study, the cumulative paw swelling was calculated for each rat as follows: a baseline value was determined by averaging the paw thickness values of day 0-9 when no signs of arthritis were visible in the vehicle-treated group. Next, increase in paw thickness was calculated by subtracting the baseline value from the paw thickness values obtained on day 10-24 (delta value). Cumulative paw swelling is defined as the sum of the delta paw thickness values from day 10 till 24.

### Histology

Left ankle joints were decalcified in a solution of 10% EDTA in distilled water, pH 7.4 for at least two months. After a wash in running tap water for 24 hours, the joints were embedded in paraffin and cut into 5 μm sections.

Joint inflammation was scored on Haematoxylin Phloxine Saffron (HPS) stained sections. The inflammation score is based on the amount of inflammatory cells infiltrated into the joint on a scale of 0-3:

0=no inflammation
1=inflammatory cells in muscle layer (surrounding tissue)
2=infiltration of inflammatory cells in the joint
3=severe infiltration of inflammatory cells in the joint

Cartilage erosion was also scored on HPS stained sections. Erosion was scored in three compartments between tarsal bones and the tibia bone as the disappearance of cartilage or chondrocyte death on a scale of 0-3:

0=no erosion
1=erosion in 1 joint compartment
2=erosion in 2 joint compartments
3=erosion in 3 joint compartments

Proteoglycan (PG) loss was scored on Saffranin 0/Fast green-stained sections on a scale of 0-3, indicating increasing loss of staining from the cartilage tissue:

0=no PG loss
1=1-33% loss of PG staining
2=34-66% loss of PG staining
3=67%-complete loss of PG staining

Bone erosion was scored on HPS stained sections. Erosion was scored at four different locations in two compartments as the number of osteoclasts per field (average value is given in Appendix 8):

0=0 osteoclasts
0.5=1-10 osteoclasts
1=11-20 osteoclasts
2=21-30 osteoclasts
3=>30 osteoclasts

### Results

Therapeutic treatment (intraperitoneal, starting on day 10) with Compound 2 at a dose of 12.6, 6.3, 3.2 and 1.6 mg/kg resulted in limited suppression of arthritis development at the highest dose level (12.6 mg/kg). This is based on the following observations:
As for the vehicle group, first clinical signs of arthritis for all Compound 2 groups were observed around day 10. Days of definite disease onset were 11.5±2.0, 10.4±0.5, 10.4±1.1 and 10.3±0.5 days (mean±SD) for the 12.6, 6.3, 3.2 and 1.6 mg/kg groups, respectively. This was not significantly different from the vehicle group.
A disease incidence of 100% was reached for all the Compound 2 groups. For both the 1.6 and 6.3 mg/kg Compound 2 group on day 11, and on day 13 and 15 for the 3.2 mg/kg 12.6 mg/kg group, respectively. Disease incidence remained 100% till the end of the study for all the Compound 2 groups.
Development of clinical signs of arthritis over time in the 6.3, 3.2 and 1.6 mg/kg Compound 2 groups was comparable to the vehicle group. For the highest dose (12.6 mg/kg) group the clinical signs of arthritis seem to be initially suppressed compared to the vehicle group, reaching significant difference for the total clinical arthritis score on day 13 and 16 (p=0.018 and p=0.011, respectively, versus vehicle. This suppression, however, was no longer observed in a later stage of disease and the total clinical arthritis score even statistically exceeded that of the vehicle group at the end of the study (p=0.036, p=0.017 and p=0.044 on day 22, 23 and 24, respectively. As a consequence, the cumulative arthritis scores for all Compound 2 groups were not significantly different from the vehicle group (77.6±19.9, 81.1±14.6, 83.3±12.2 and 77.3±11.9 for the 12.6, 6.3, 3.2 and 1.6 mg/kg groups, respectively, versus 84.1±11.9 a.u. (mean±SD) for the vehicle group.
A reduction of the cumulative hind paw swelling was observed in the highest dose (12.6 mg/kg) group throughout the study. This reduction was significantly different compared to the vehicle group (p=0.003 and p=0.004 for the left and right hind paw, respectively). For the other Compound 2 dose groups no significant differences for hind paw swelling were observed.
Consistent with these findings, the four parameters scored in the histopathological analysis, demonstrated that the left ankles joints in the 6.3, 3.2 and 1.6 mg/kg Compound 2-treated rats were arthritic. At the highest dose (12.6 mg/kg) group only a significant suppression on proteoglycan loss was observed (p=0.026 versus vehicle). Mean values±SD for joint inflammation: 2.41±0.42, 2.69±0.32, 2.66±0.33 and 2.69±0.37 for 12.6, 6.3, 3.2 and 1.6 mg/kg groups, respectively. Mean values±SD for cartilage erosion: 1.81±0.91, 2.06±0.65, 2.34±0.68 and 2.09±0.65 for 12.6, 6.3, 3.2 and 1.6 mg/kg groups, respectively. Mean values±SD for proteoglycan loss: 1.97±0.59, 2.47±0.34, 2.53±0.21 and 2.41±0.40 for 12.6, 6.3, 3.2 and 1.6 mg/kg groups, respectively. Mean values±SD for bone erosion: 0.56±0.14, 0.69±0.13, 0.62±0.09 and 0.63±0.17 for 12.6, 6.3, 3.2 and 1.6 mg/kg groups, respectively.
Body weight of the rats treated with the different concentrations Compound 2 decreased gradually after disease onset, which was similar to the vehicle group.

### Conclusion

All vehicle-treated rats in this Example developed severe arthritis, indicating a successful induction of arthritis. Dexamethasone (0.5 mg/kg i.p.) treatment in a therapeutic setting resulted in a suppression of the cumulative arthritis score of approximately 97%. In addition, hind paw swelling was significantly diminished. These observations demonstrate that the model was sensitive to treatment.

Therapeutic treatment with different concentrations Compound 2 (12.6, 6.3, 3.2 and 1.6 mg/kg), had no significant effect on the cumulative arthritis scores compared to the vehicle control group. However, hind paw swelling, measured by a laser scan micrometer, was significantly reduced in the highest dose group (12.6 mg/kg). Furthermore, in the initial phase of the disease a suppressive effect on the total clinical score was observed in this group, reaching a significant difference on day 13 and 16 compared to the vehicle group. However, this effect disappeared in the later stage of the disease and even statistically exceeded the total clinical score of the vehicle group from day 22 onward, explaining the lack of effect on cumulative arthritis score. Moreover, loss of proteoglycans in the ankle joint was significantly diminished in the 12.6 mg/kg group. At lower dose levels of Compound 2 no significant effects on these parameters were observed.

In summary, a limited effect of the highest dose level of Compound 2 on arthritis development was demonstrated in the study with significant reduction of the total clinical arthritis score on day 13 and 16, hind paw swelling and proteoglycan loss in the ankle joint. Other arthritis parameters were not significantly affected by the treatment with Compound 2.

### Example 10

### Human M1/M2 Macrophage Skewing after Incubation with TLR7 Agonists

To extend and validate the tolerogenic and polarizing action of Compound 2 on human cells, the effects of Compound 2 have been analyzed in monocytes, peripheral blood mononuclear cells, and monocyte-derived macrophages, obtained from at least four different healthy donors.

Experimental Plan:

To evaluate the effects of TMX on human cell activation, monocytes were isolated from buffy coats obtained from four different healthy donors. With respect to macrophage tolerance, cells were treated with Compound 2 (10 microM) up to 20 hours and then re-challenged with agonists/ligands of different TLRs (TLR2, TLR4, TLR5, TLR9, TLR7, etc. . . . ), including the TLR7 unconjugated ligand 1V209 (free pharmacophore) at a concentration of 10 microM.

TMX-mediated tolerance was promoted by the following scheme (M=medium)

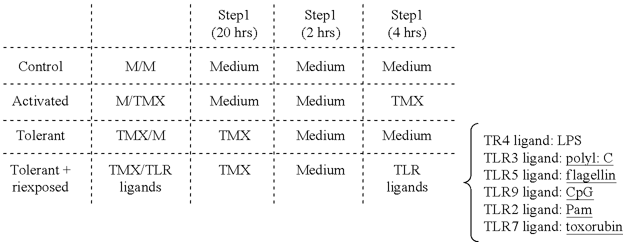

Materials and Methods:

Cell Culture and treatments. Monocytes (Mo) were obtained from buffycoats collected from healthy donors as previously described (Porta C et al, PNAS 2009). Cells were incubated in RPMI 1640 medium, containing 10% fetal bovine serum, 2 mM glutamine and 100 U/ml of penicillin-streptomycin. The concentrations for the different treatments were as follows: LPS (100 ng/ml) (Lipopolysaccharide from Salmonella Abortus Equi S-form, Alexis), CpG (1 microg/ml), Pam3CSK (2 microg/ml), Poly I:C (10 microg/ml), Loxorubine (100 microM), flagellin (100 ng/ml). To induce TMX tolerance, cells were incubated in the presence of TMX (10 microM) for 20 hrs, washed and maintained in RPMI medium for 2 hrs and then re-challenged with TMX (10 microM) for 4 hrs. To induce TMX-tolerization (T/M) cells were treated with TMX for 20 hrs, washed and then maintained in medium for additional 6 hours, without TMX re-challenge. To induce M1-activation cells were incubated in medium for 20 hrs, washed, left in medium for 2 hrs and finally stimulated with TMX (10 microM) for 4 hrs. Control cells (M/M) were cultured in medium for the entire period of the experiment. For gene expression analysis, total RNA was extracted from TMX treated and untreated cells with trizol (Invitrogen), according to the manufacturer's instructions. To analyze cytokines and chemokines production supernatants were collected 24 hrs after the second challenge with TMX.

Real-Time PCR. Total RNA was purified as previously described. Briefly, reverse transcription from 1 microgram of RNA was performed using a cDNA Archive kit (Applied Biosystem, NJ, USA). Real time PCR was performed using Power Syber Green PCR Master Mix (Applied Biosystem, NJ, USA) and detected by 7900HT Fast Real-Time System (Applied Biosystem, NJ, USA). Data were processed using the SDS2.2.2 software (Applied Biosystem, NJ, USA). Results were normalized to the expression of the housekeeping gene 8-actin and then expressed as folds of upregulation, with respect to the control cell population.

ELISA. Cell-free supernatants were tested by ELISA for the indicated cytokines/chemokines. Human TNF-alpha, IL-6, IL-1beta, IL-10, CCL17 and CCL22 ELISA kits were purchased from R&D Systems (Minneapolis, Minn.).

In Vitro Effects of Compound 2 on Human Monocyte Activation:

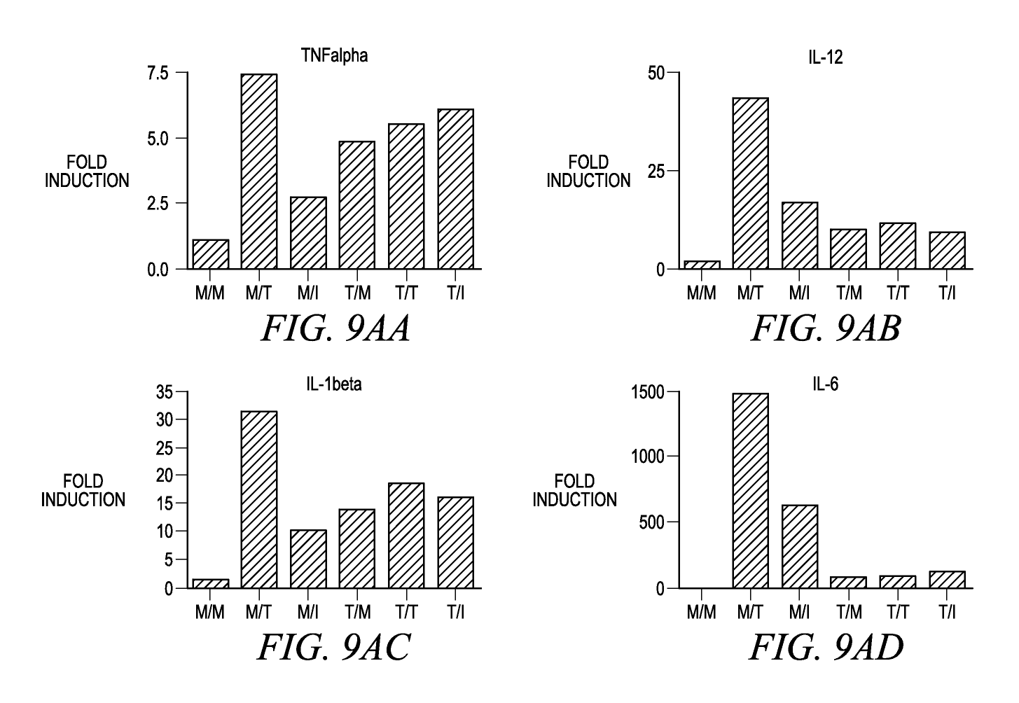

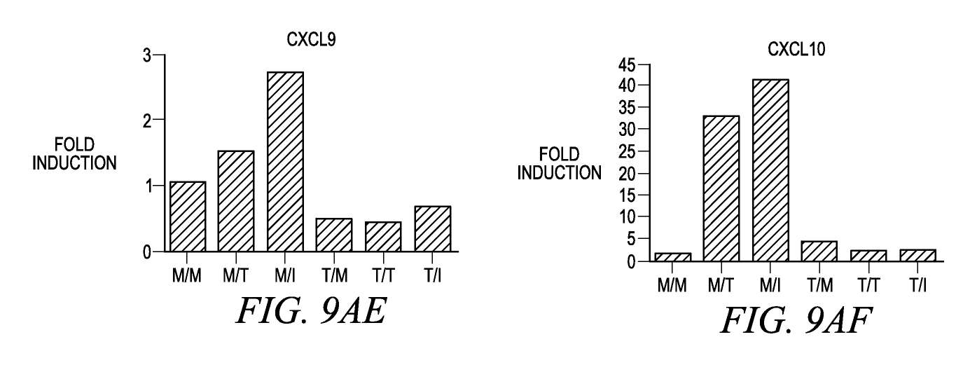

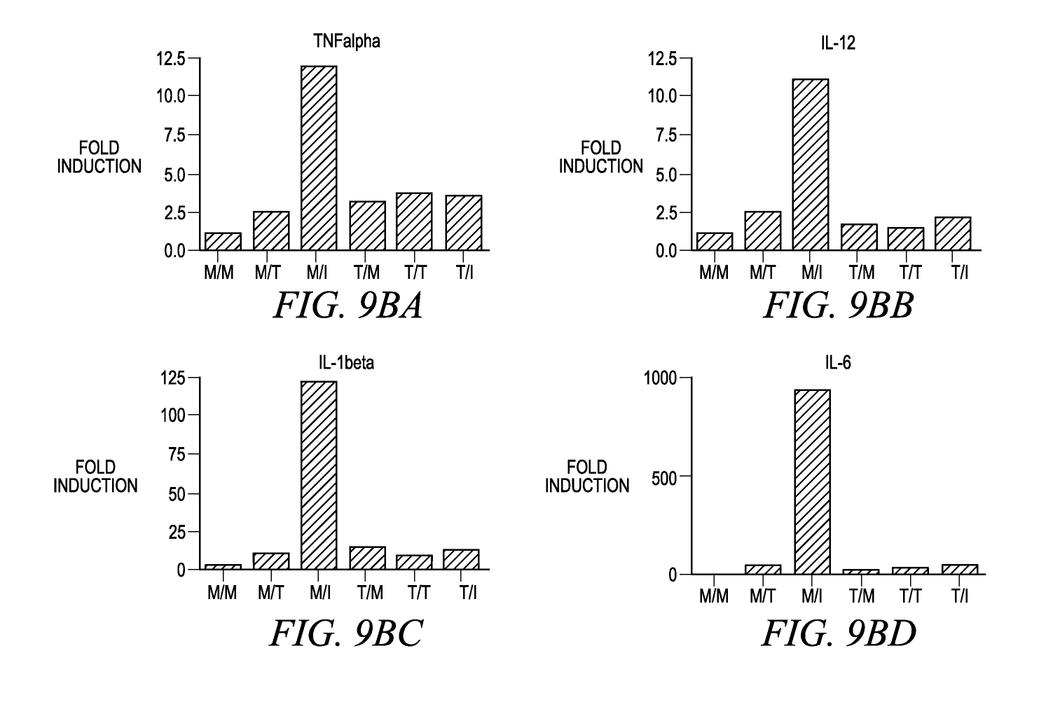

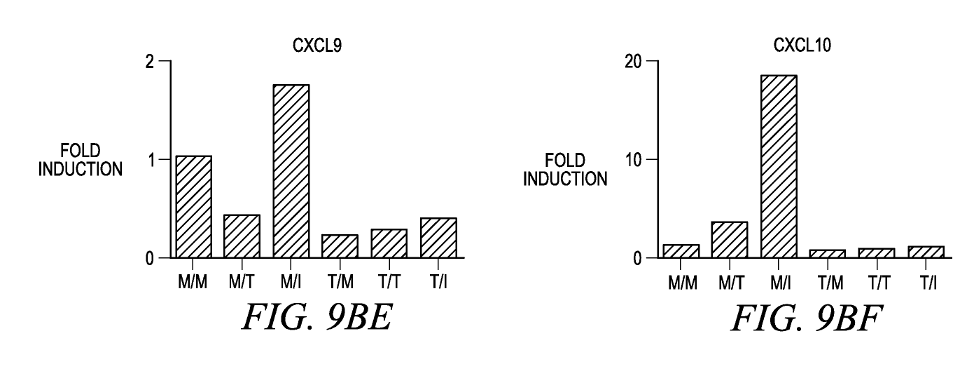

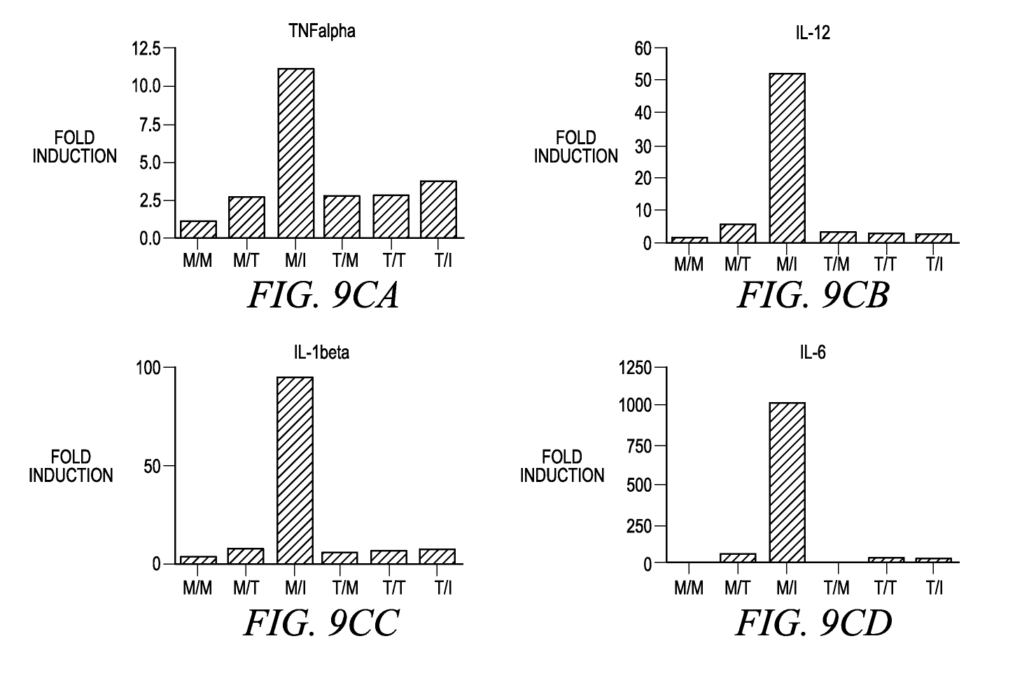

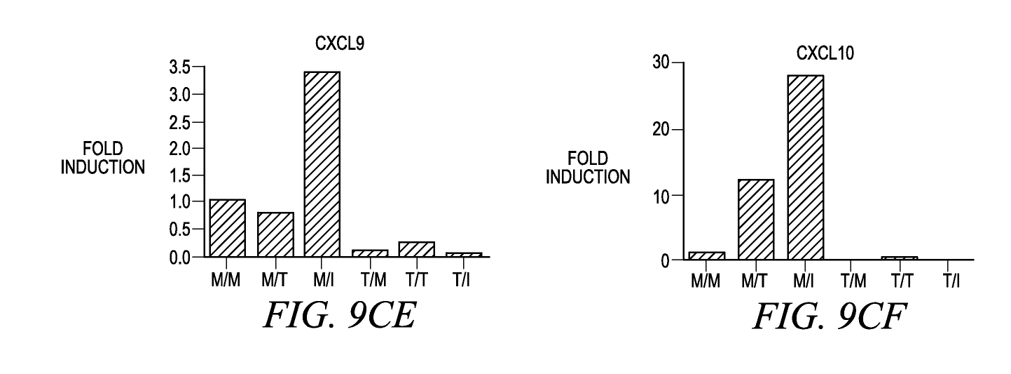

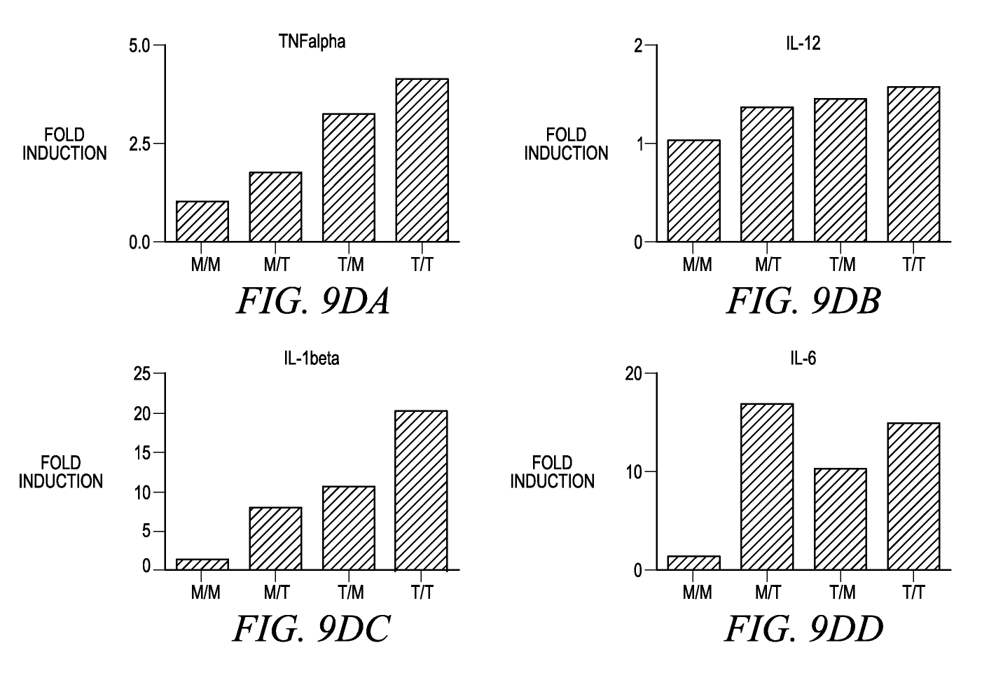

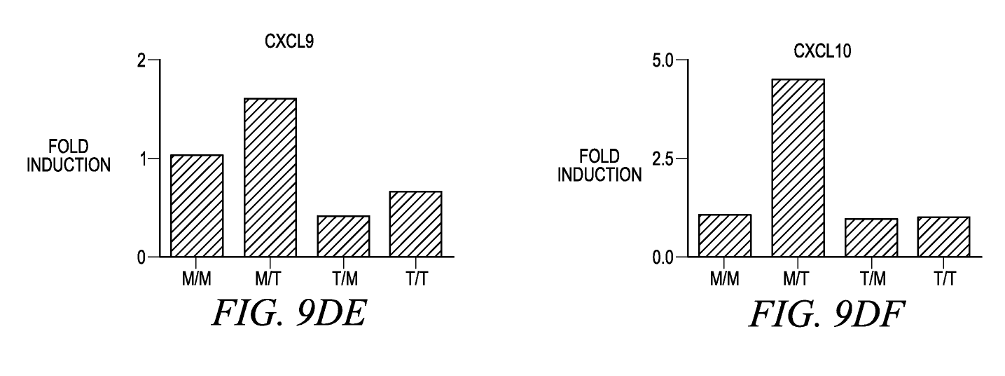

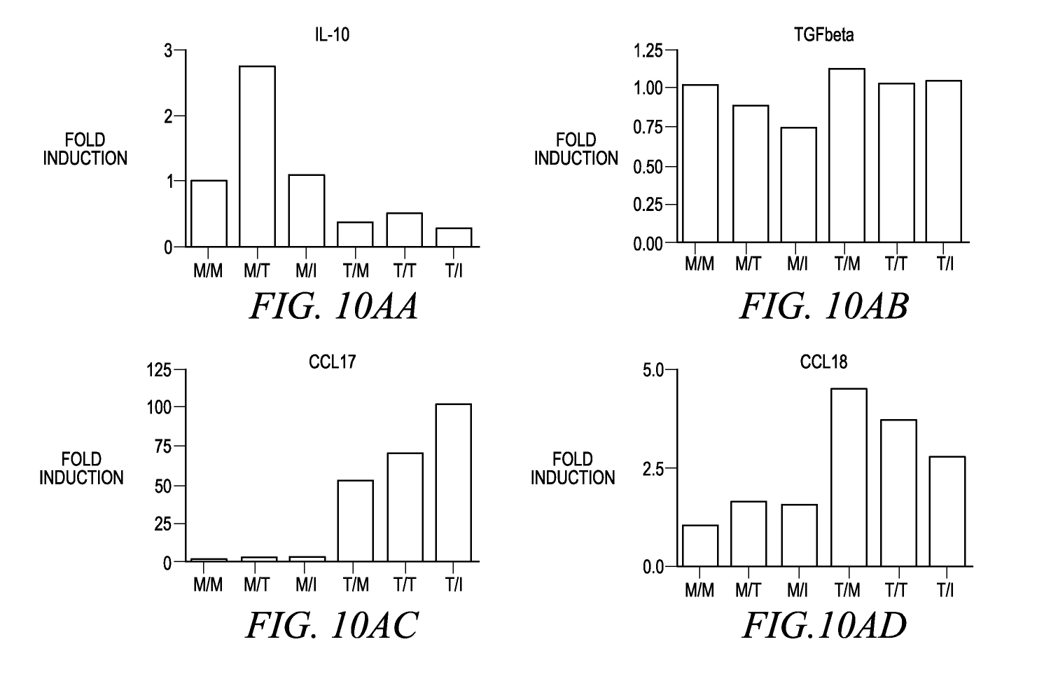

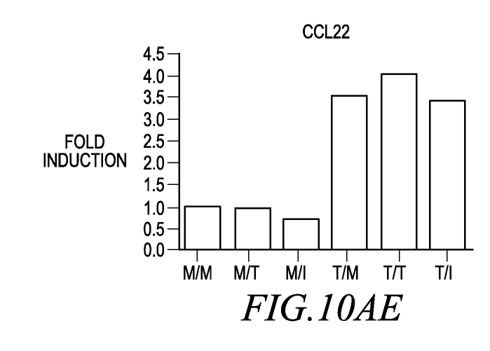

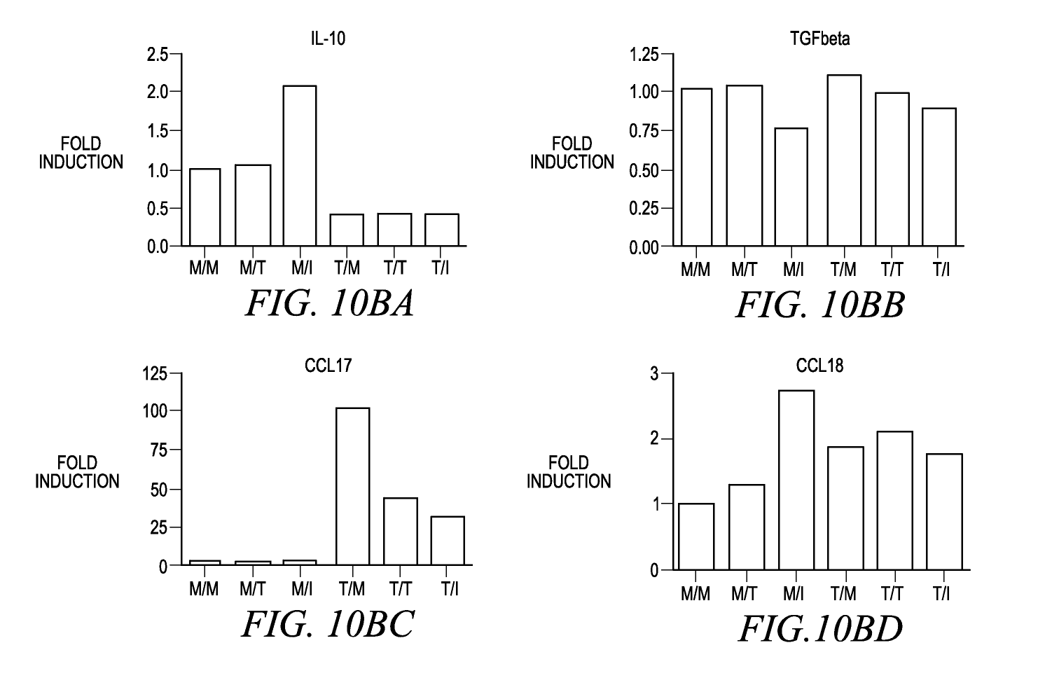

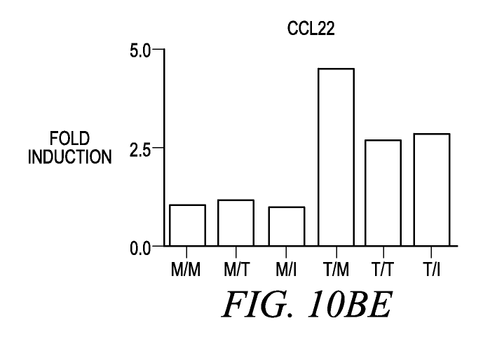

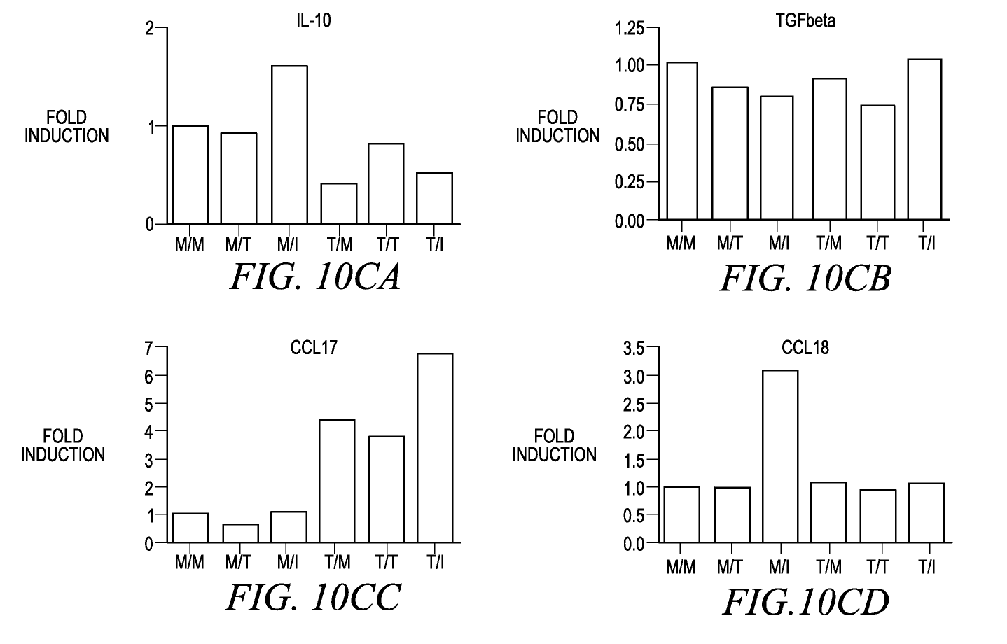

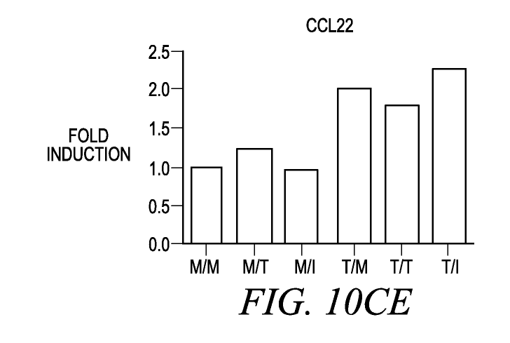

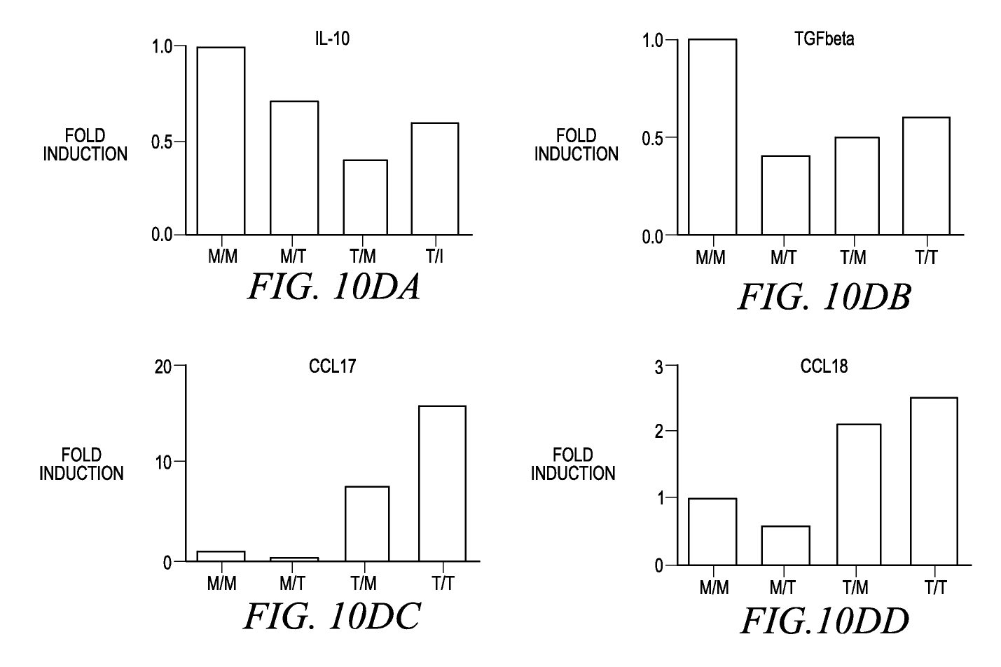

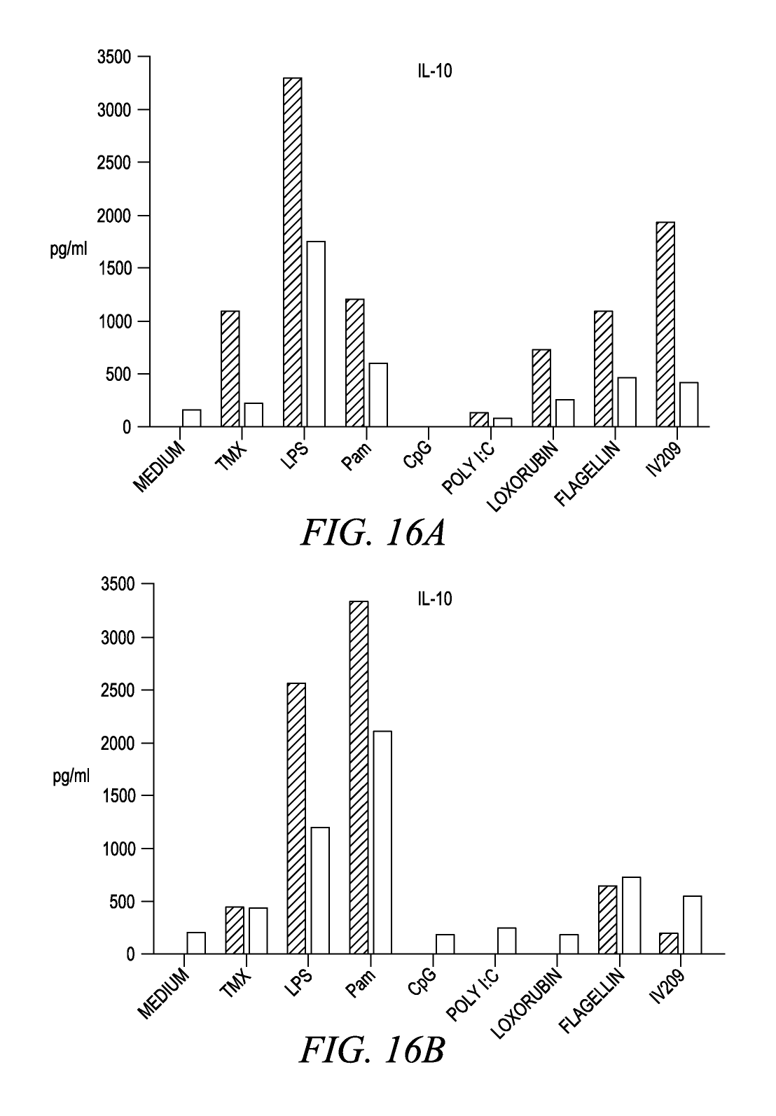

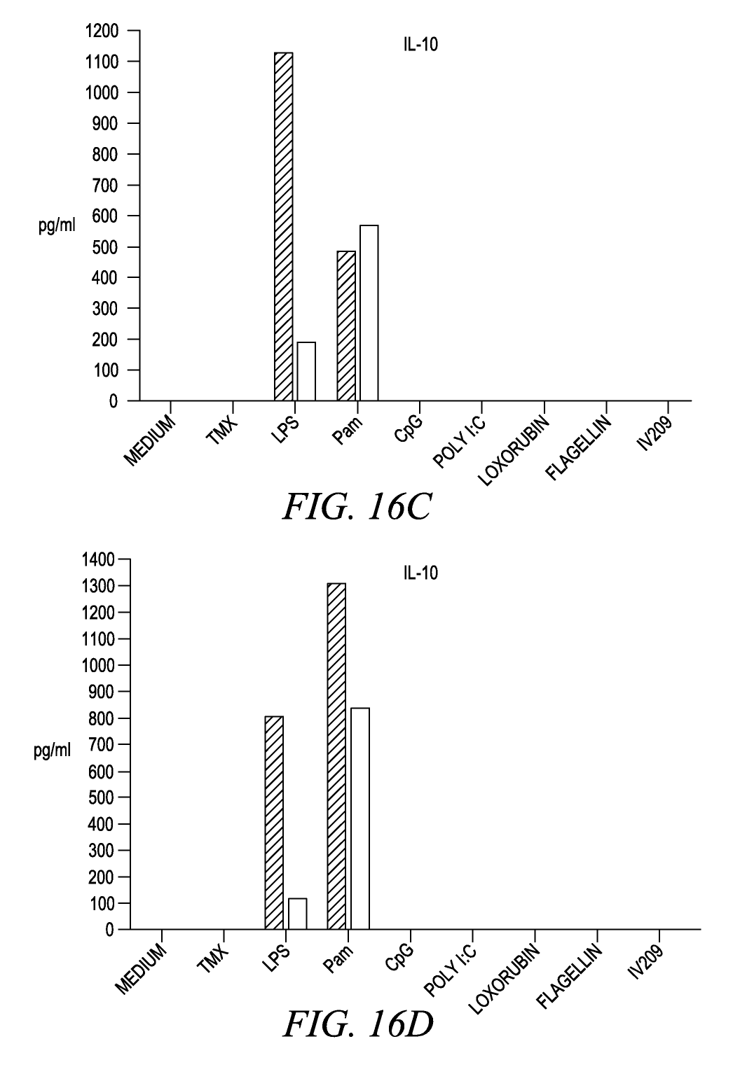

RNA extracted from control (M/M), Compound 2 activated (M/T), 1V209 activated (M/I), tolerant (T/M) and tolerant Mo, re-challenged with Compound 2 (T/T) or with 1V209 (T/I), were analyzed by RT PCR for the expression of representative M1 and M2 genes. The results showed that TMX induced a state of tolerance characterized by low M1 (TNF-alpha, IL-1-beta IL-6, IL-12, CXCL10) gene expression in Mo obtained from 3 out of 4 different healthy donors (FIG. 9). Further, with a few exceptions shown in panels C(CCL18) and D (CCL22), the M2 genes CCL17, CCL18, CCL22 are further induced in tolerant (TfT and T/I) than in activated (M/T and M/I) Mo (FIG. 10). IL-10 expression is inhibited in tolerant Mo as compared to activated Mo (FIG. 10) No significant modulation of the M1 markers CXCL9 (FIG. 9, IFNbeta, iNOS and the M2 cytokine TGFbeta (FIG. 16), was observed. Excluding the Mo from one healthy donor (FIG. 9A, 1V209 agonist showed an higher potency as compared to Compound 2 in terms of induction of both M1 and M2 gene expression.

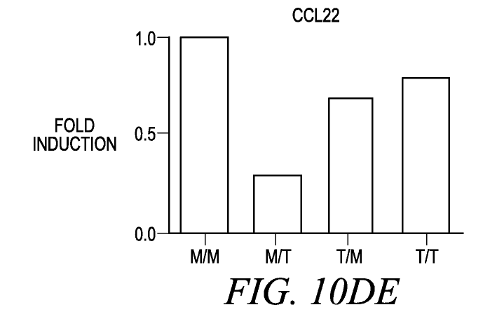

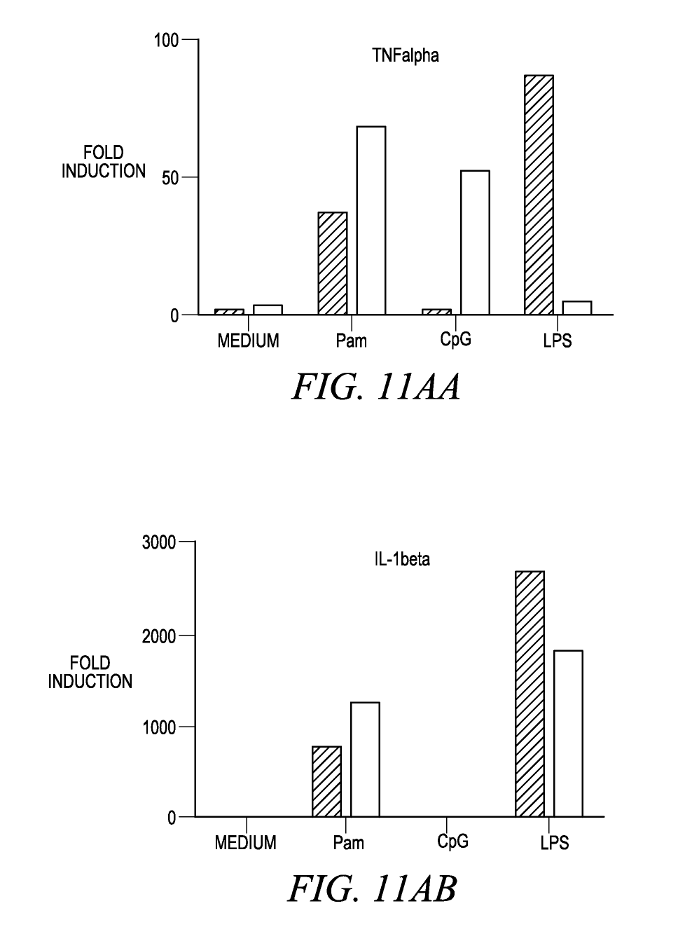

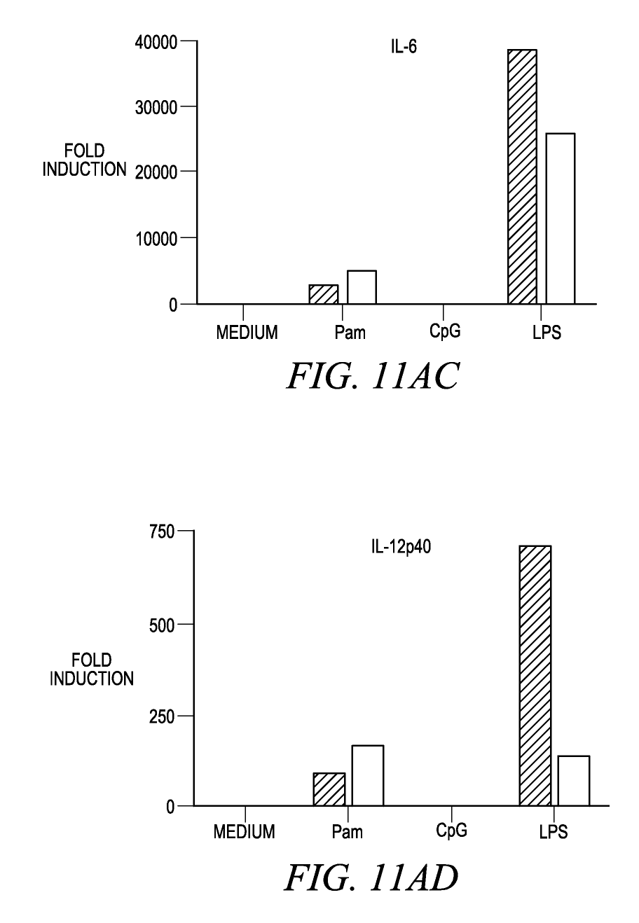

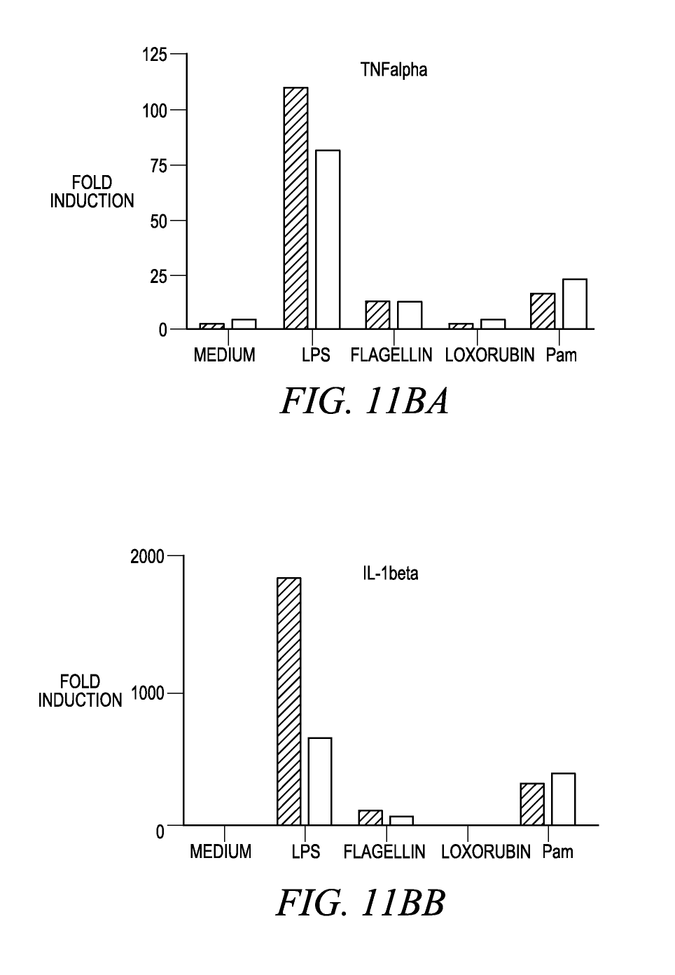

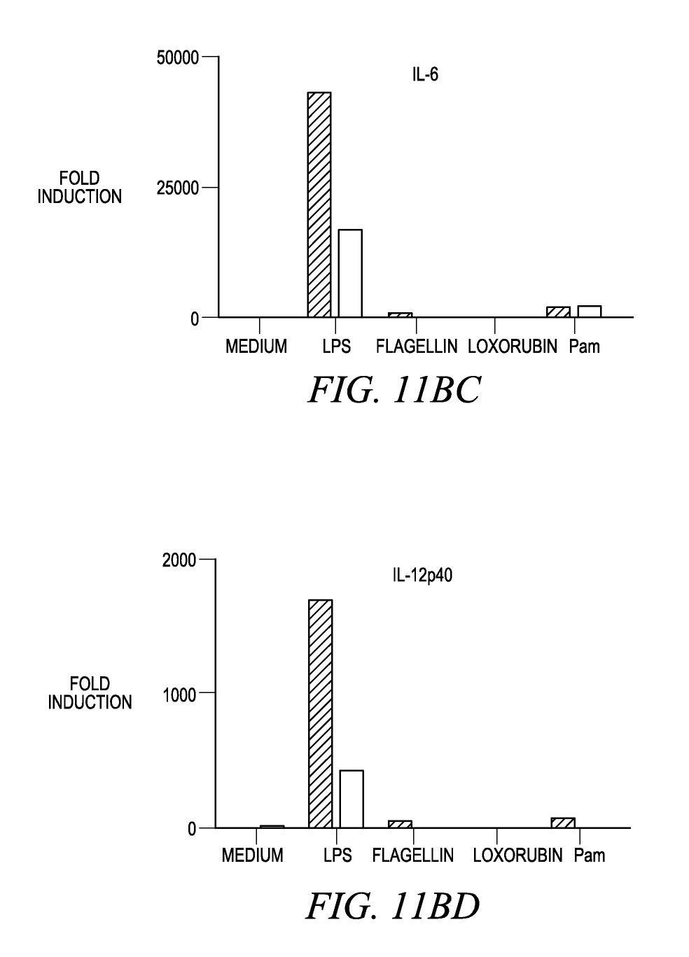

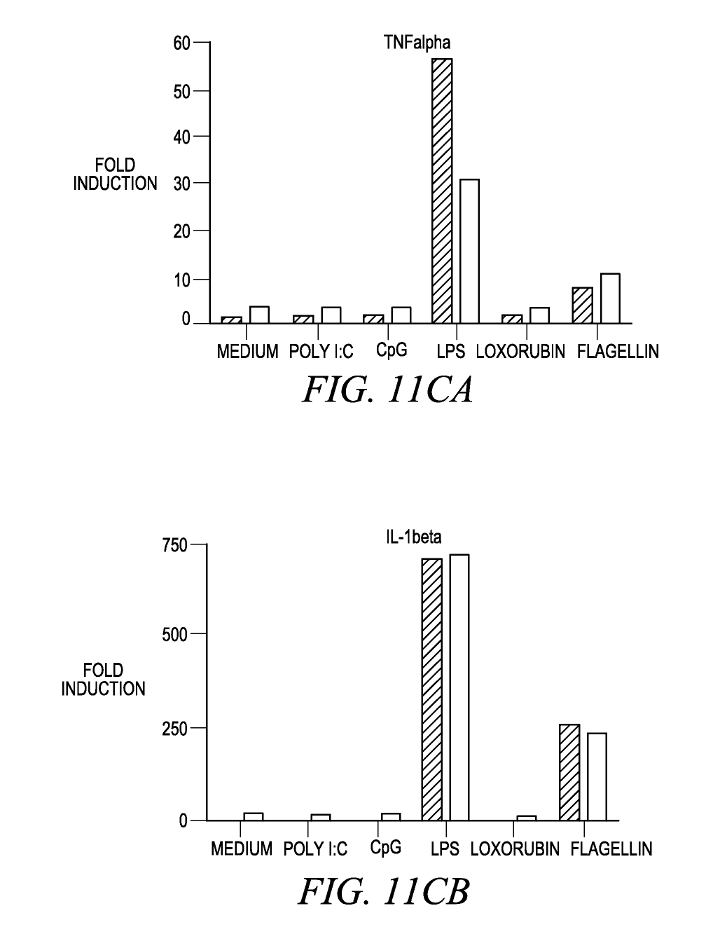

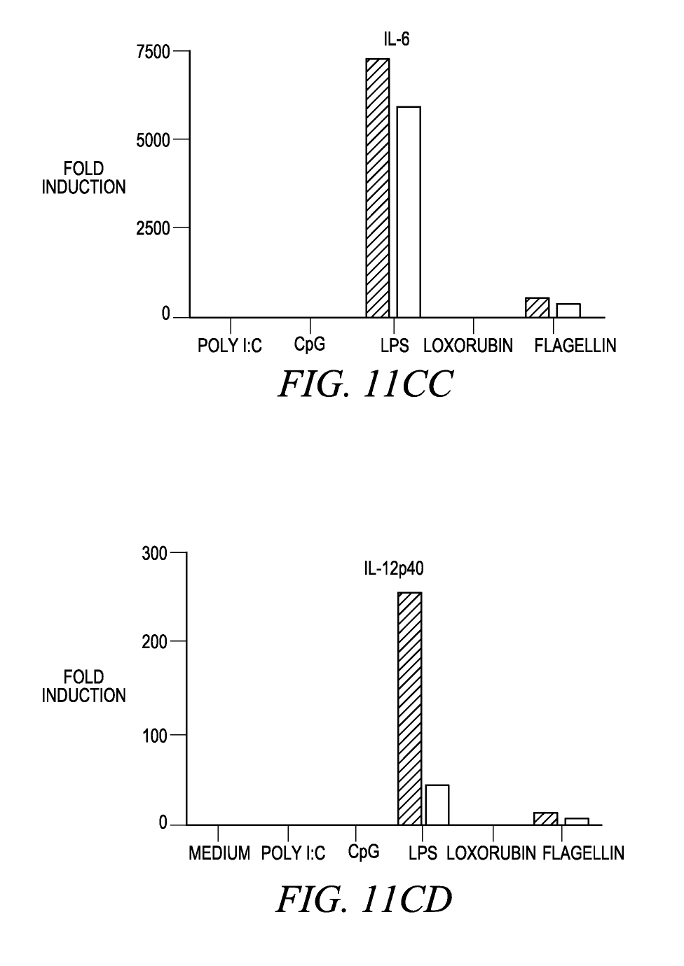

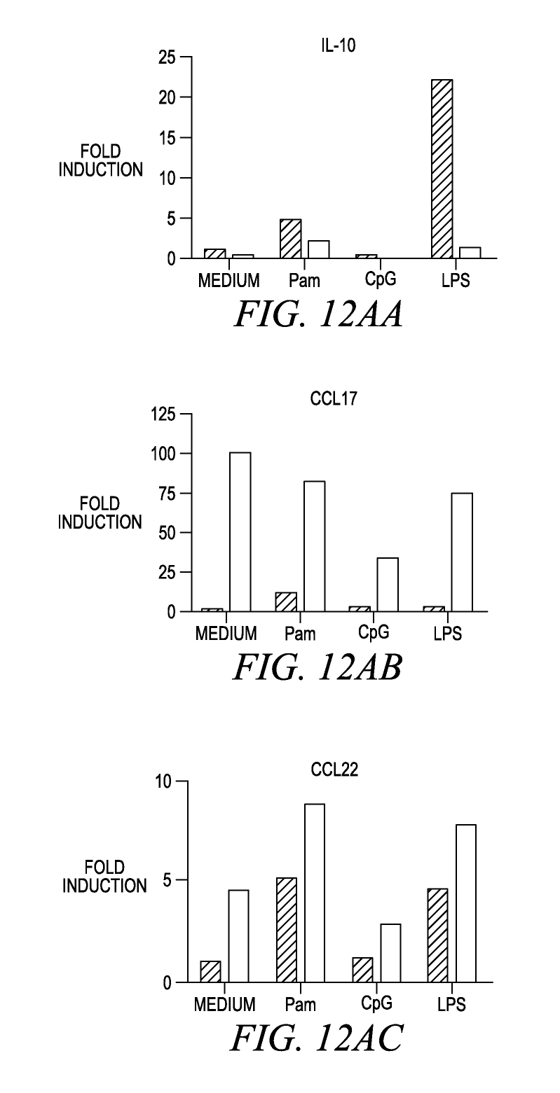

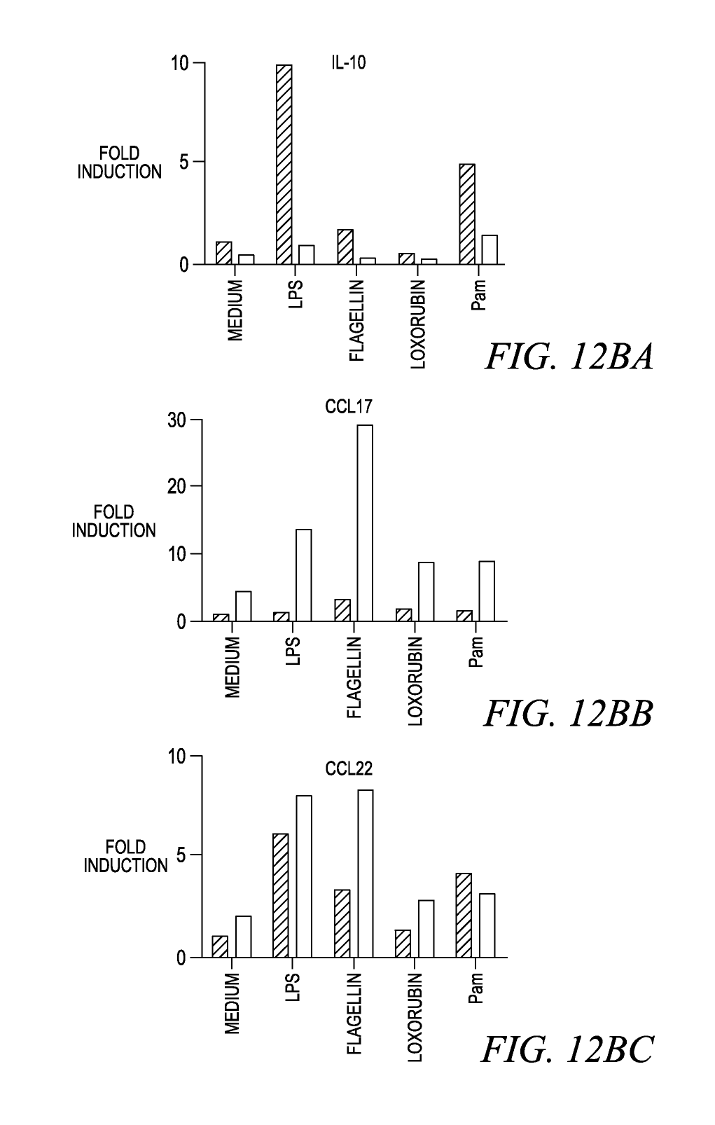

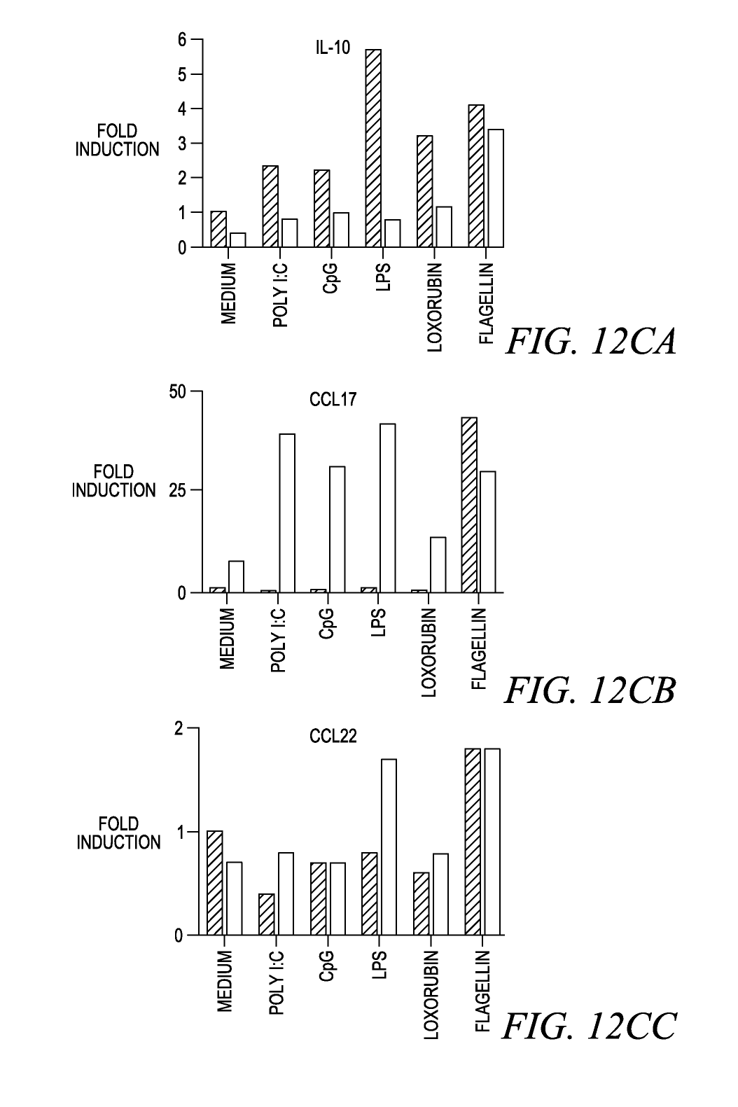

The capacity of Compound 2 to promote cross tolerance toward several TLR ligands was also evaluated. The results showed that, excluding Pam, Mo tolerized by 20 hours pre-treatment with Compound 2 (white bars) maintained the inability to up-regulate TNFalpha, IL-1 beta, IL-6 and IL-12 (FIG. 11) gene expression in response to TLR ligands. In contrast all the different TLR engagements trigger the expression of Th2-recruiting chemokines genes (CCL17, CCL22) but not IL-10 (FIG. 12). There are some exceptions for CpG (FIG. 11A) and flagellin (11C; 12C), in Mo from one donor.

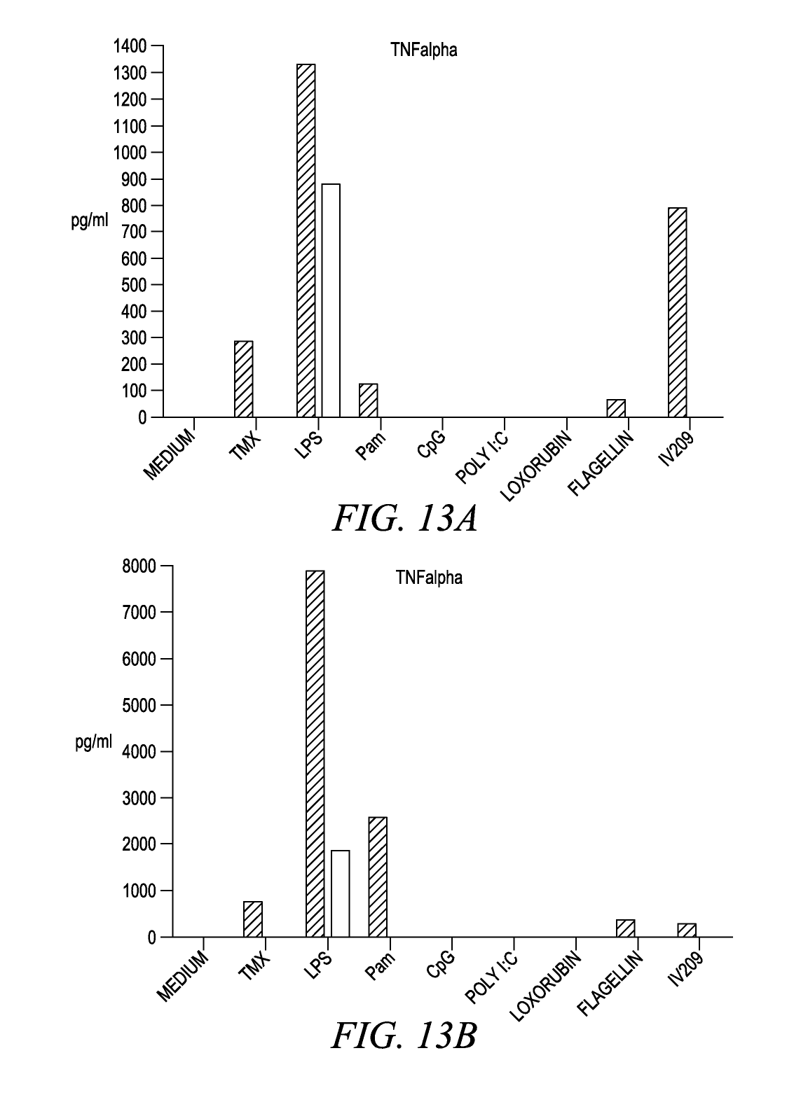

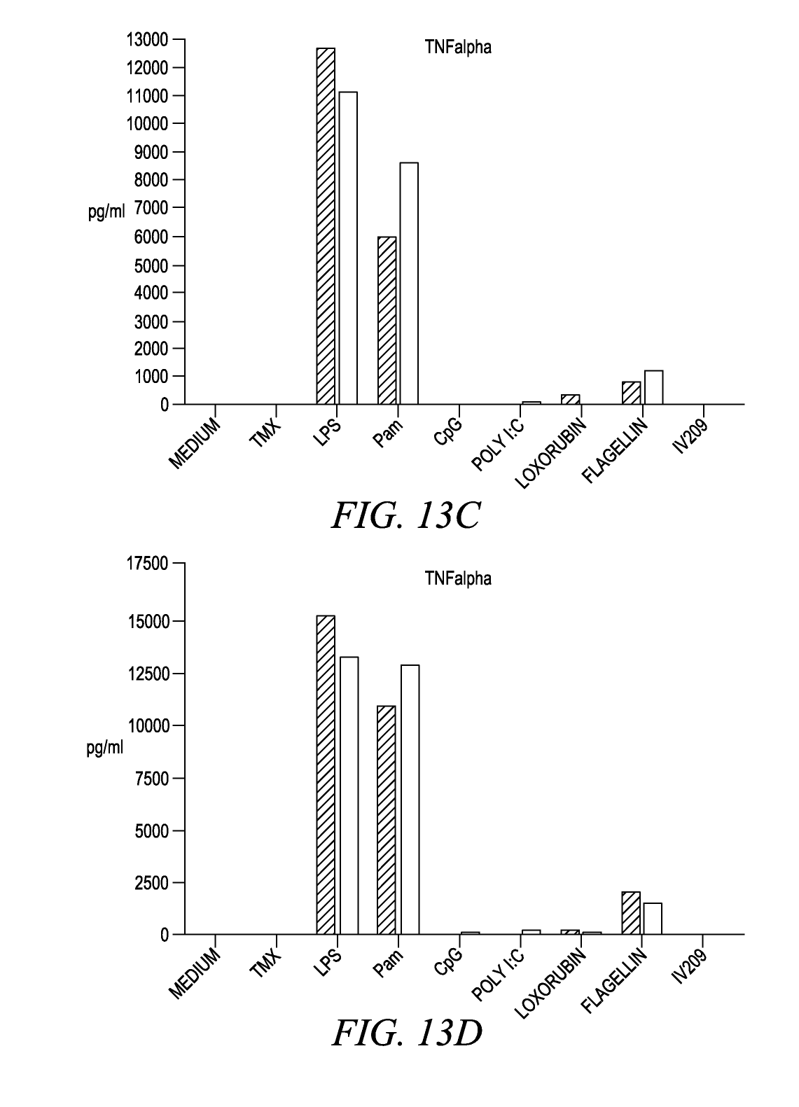

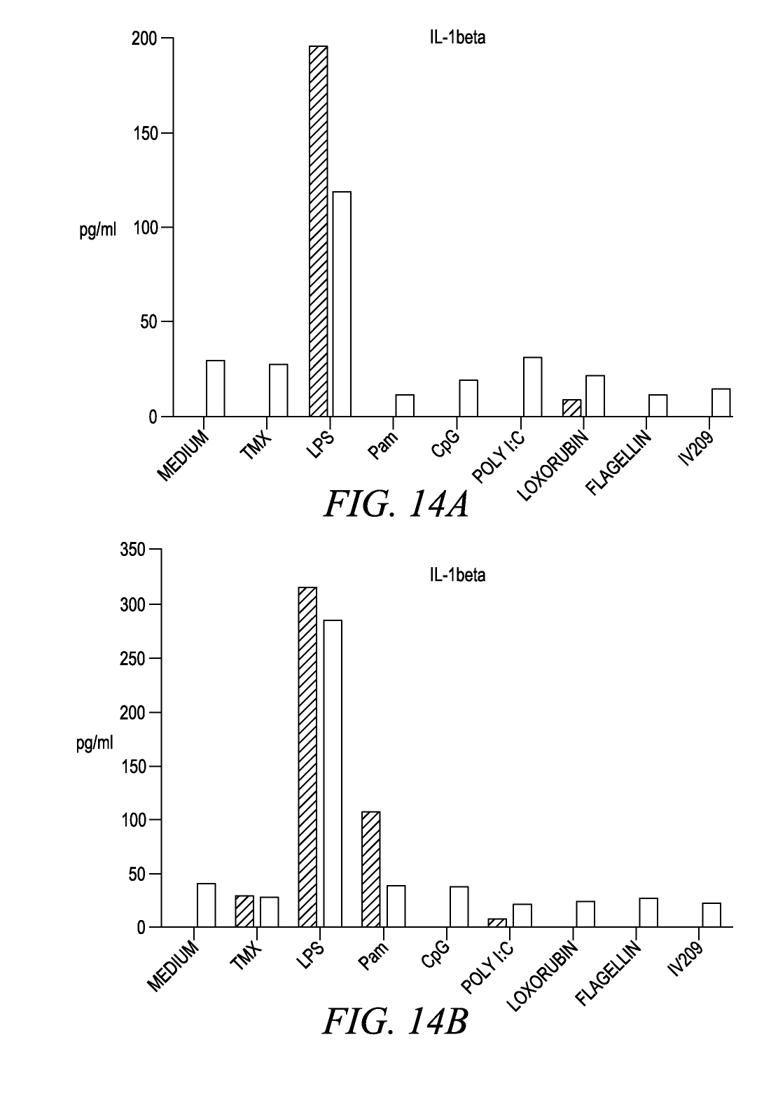

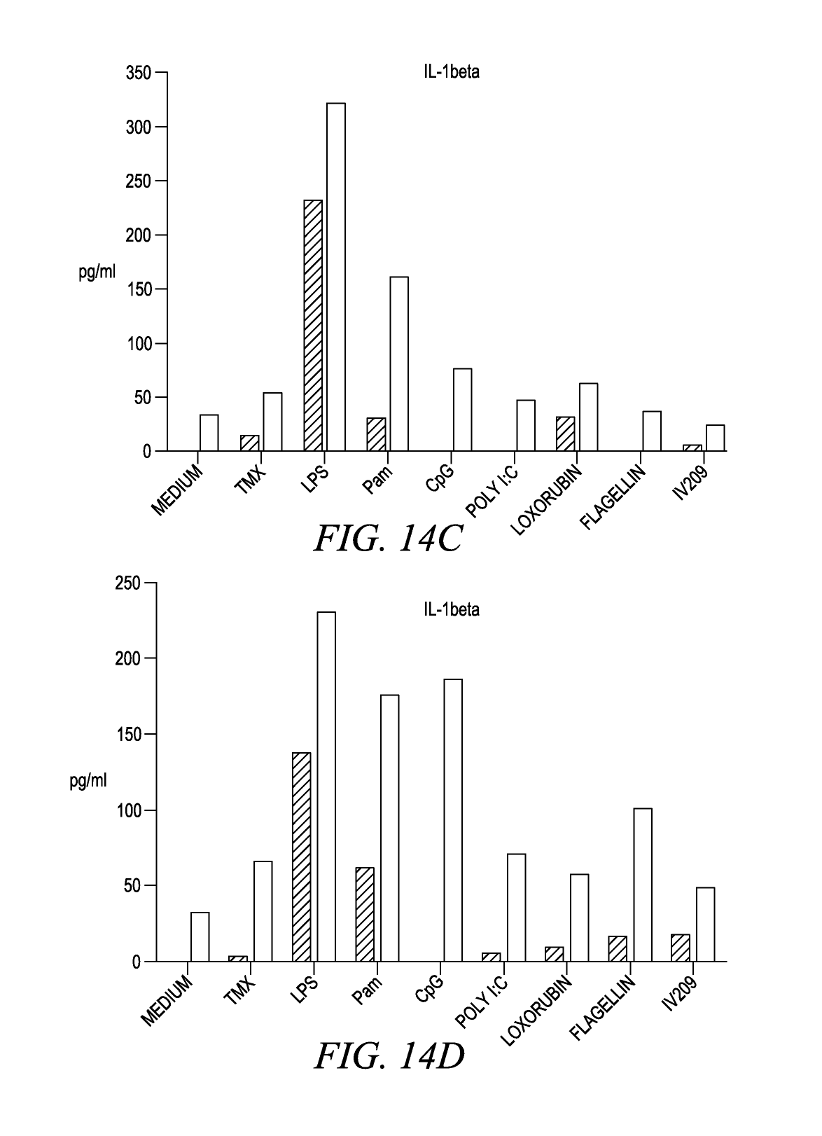

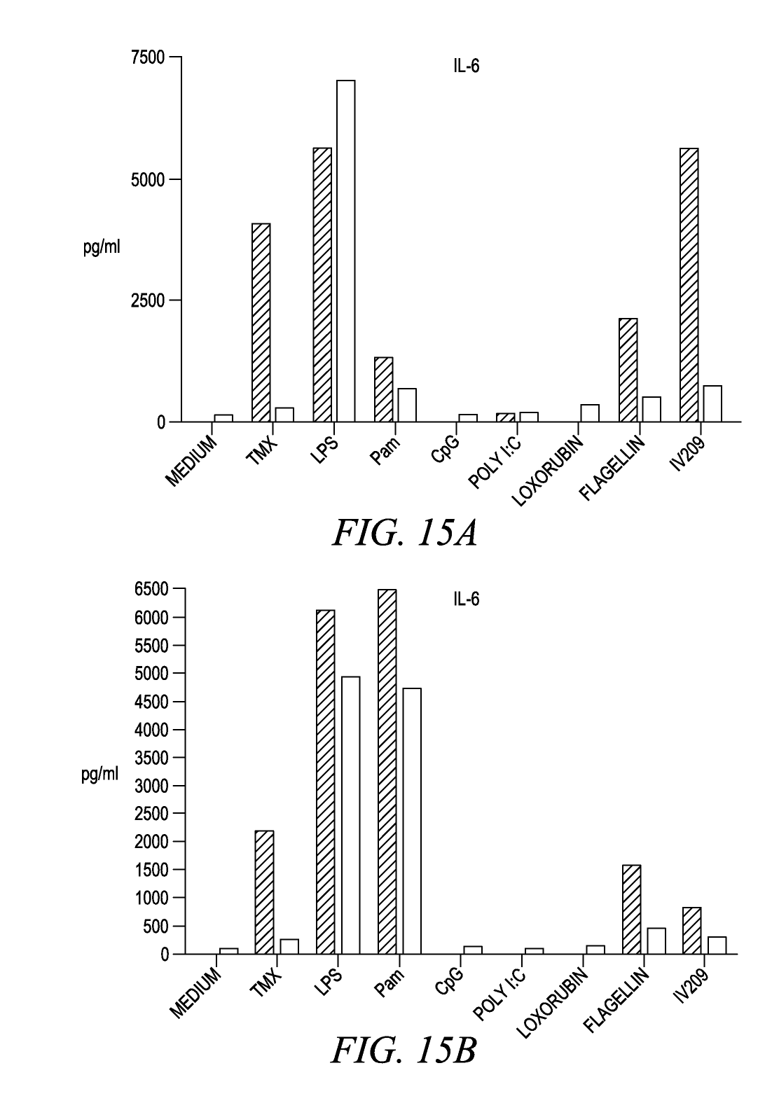

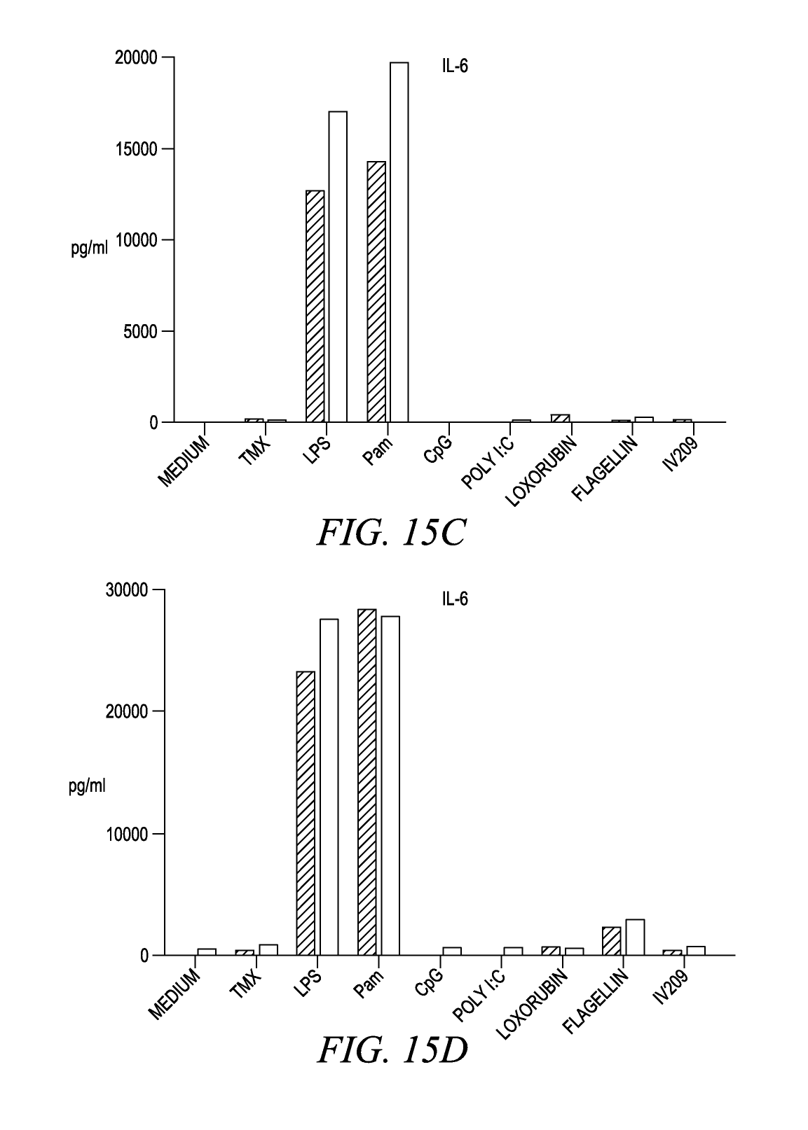

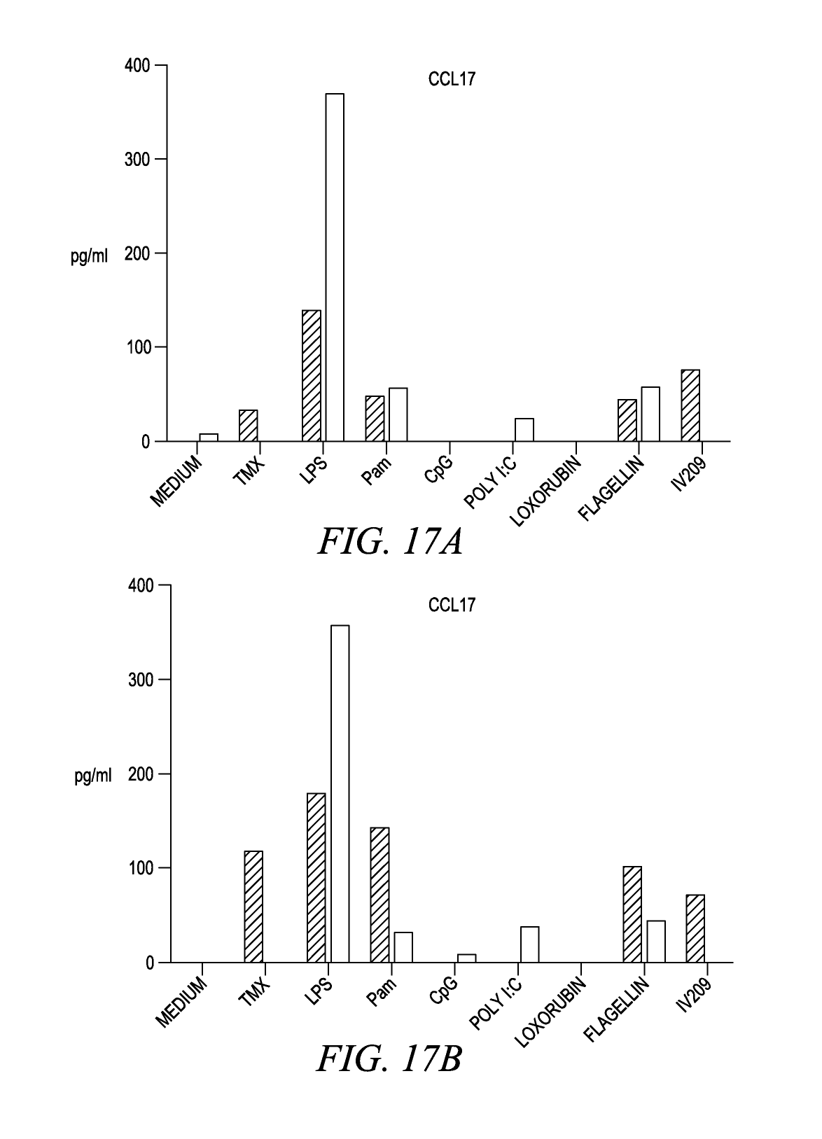

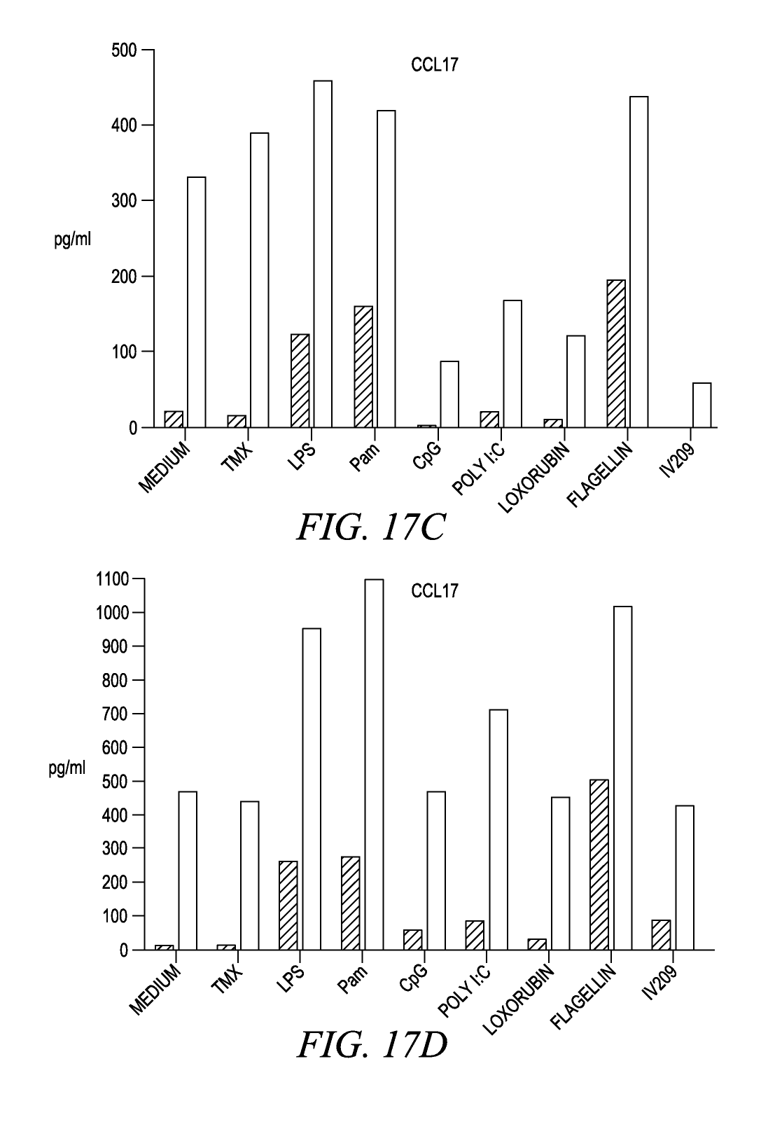

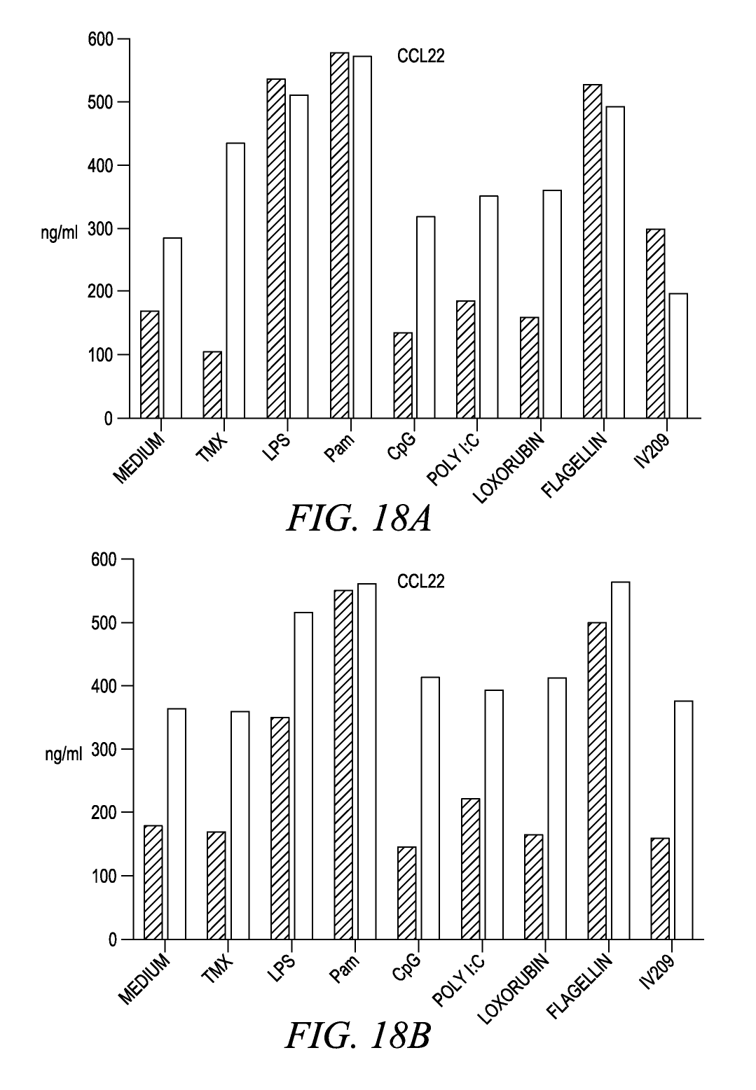

To further investigate the results obtained by gene expression analysis, the secretion of selected M1 and M2 gene products by TMX-tolerant monocytes was analyzed. According to transcript levels, the results confirmed the inhibition of TNFalpha (FIG. 13) and IL-6 (FIG. 15) secretion and the induction of CCL17 (FIG. 17) and CCL22 (FIG. 18) in TMX-tolerized cells (white bars) re-challenged with TMX or 1V209. Concerning the capacity of TLR ligands to induce pro-inflammatory cytokines production in TMX-tolerized cells, the capacity of Pam to induce M1 cytokines was confirmed (FIG. 13, 14, 15) and it was observed that all TLR agonists promote IL-1beta production (FIG. 14). Except for LPS, in agreement with gene expression analysis, both TNF and IL-6 secretion are decreased in cells tolerized with TMX and re-challenged with TLR agonists (FIG. 13, 15). The secretion of selected M2 markers (CCL17 and CCL22) was significantly augmented in TMX-tolerized Mo from 2 out of 4 different healthy donors (FIG. 17, 18). Levels of IL-10 in the supernatant of TMX-tolerant monocytes were decreased (white bars) (FIG. 16).

The M1 markers TNF-alpha, IL12-p40 IL-1 IL6 CXCL10 and IFNbeta were down-regulated in the presence of TMX pretreatment (FIG. 9). M2 signature chemokines CCL17, CCL18 and CCL22 were increased in the presence of TMX compound (FIG. 10). The tolerant effect induced by TLR7 partial agonist pretreatment was also efficacious when stimulating through TLRs other than TLR7. The M1 marker IL-12p40 was considerably down-regulated—approx by 10 fold-by TMX pretreatment (FIG. 11). Further, the M2 gene induction by TMX treatment was strongly enhanced, CCL17 in particular (see FIG. 12, middle panel).

The main difference observed in humans with respect to the mouse data is the effect of Compound 2 treatment on IL-10 production: Compound 2-conditioned cells produce an increased 11-10 amount. The plasticity and differentiation of macrophages into M1 and M2 functional phenotypes represent extremes of a continual spectrum of differential pathways. Various subtypes of M2 macrophages were described, showing peculiar phenotypic functions. IL-10 is differentially expressed in these subpopulations, being up-regulated in some but not all of them. Thus the differences in M2 marker genes observed between various sources of mouse macrophages and human monocytes may be ascribed to the different cellular sources and in vitro conditions used, but the M2 skewing signature determined by Compound 2 is confirmed.

Overall the results suggest that Compound 2 has a partial tolerizing and polarizing activity on human cells as compared to murine cells. 20 hours of pre-treatment with Compound 2 is able to inhibit M1 (TNFalpha, IL-6, IL-12, CXCL9) gene expression and pro-inflammatory cytokines (TNFalpha, IL-6) production in monocytes.

Whereas several TLR ligands are unable to induce IL-12 genes expression in TMX-tolerized cells, they trigger a higher IL-1beta production in monocytes pre-treated with TMX for 20 hours as compared to cells maintained in medium. Further, in monocytes, LPS and Pam seem to be able to break the tolerance in terms of TNFalpha and IL-6 production.

The Th2-recruiting chemokines (CCL17 and CCL22) are highly expressed and produced by TMX-tolerized cells and further induced by different TLR ligands, but decreased level of the anti-inflammatory cytokines IL-10 was observed in cells pre-treated with Compound 2 for 20 hours and re-challenged with TLR-agonists.

### REFERENCES

Porta C, Rimoldi M, Raes G, Brys L, Ghezzi P, Di Liberto D, Dieli F, Ghisletti S, Natoli G, De Baetselier P, Mantovani A, Sica PNAS, 2009; 106(35):14978-83
Medvedev A E, Sabroe I, Hasday J D, Vogel S N. J Endotoxin Res. 2006; 12(3):133-50.
Mantovani A, Sica A, Locati M. Immunity. 2005; 23:344-346.
Cros J, Geissmann F. Immunity. 2010 33(3):375-86)
Miettinen M et al. J Leukoc Biol. 2008 84(4):1092-100
Gantier M P et al. J. Immunol. 2008 Feb. 15; 180(4):2117-24
Ricardo S D, et al. 2008. J Clin Invest. 118:3522-3530
Martinez, F. O., Sica, A., Mantovani, A., and Locati, M. 2008. Front. Biosci. 13:453-461.
Mantovani, A., et al. 2004. Trends Immunol. 25:677-686.

The entirety of each patent, patent application, publication and document referenced herein hereby is incorporated by reference. Citation of the above patents, patent applications, publications and documents is not an admission that any of the foregoing is pertinent prior art, nor does it constitute any admission as to the contents or date of these publications or documents.

Modifications may be made to the foregoing without departing from the basic aspects of the technology. Although the technology has been described in substantial detail with reference to one or more specific embodiments, those of ordinary skill in the art will recognize that changes may be made to the embodiments specifically disclosed in this application, yet these modifications and improvements are within the scope and spirit of the technology.

The technology illustratively described herein suitably may be practiced in the absence of any element(s) not specifically disclosed herein. Thus, for example, in each instance herein any of the terms “comprising,” “consisting essentially of,” and “consisting of” may be replaced with either of the other two terms. The terms and expressions which have been employed are used as terms of description and not of limitation, and use of such terms and expressions do not exclude any equivalents of the features shown and described or portions thereof, and various modifications are possible within the scope of the technology claimed. The term “a” or “an” can refer to one of or a plurality of the elements it modifies (e.g., “a reagent” can mean one or more reagents) unless it is contextually clear either one of the elements or more than one of the elements is described. The term “about” as used herein refers to a value within 10% of the underlying parameter (i.e., plus or minus 10%), and use of the term “about” at the beginning of a string of values modifies each of the values (i.e., “about 1, 2 and 3” is about 1, about 2 and about 3). For example, a weight of “about 100 grams” can include weights between 90 grams and 110 grams. Thus, it should be understood that although the present technology has been specifically disclosed by representative embodiments and optional features, modification and variation of the concepts herein disclosed may be resorted to by those skilled in the art, and such modifications and variations are considered within the scope of this technology.

Certain embodiments of the technology are set forth in the claim(s) that follow:

1. A method for treating a condition in a subject, comprising administering to the subject a compound having a structure according to Formula I: 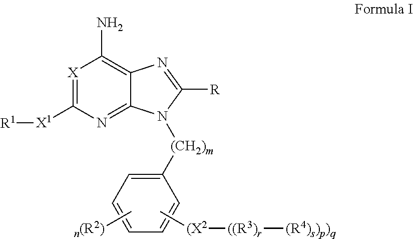
or a pharmaceutically acceptable salt thereof, including a hydrate thereof, wherein:
X is N or CR²;
R is —OR¹, —SR¹, or —NR^{a}R^{b},
X¹ is a bond or is —O—, —S—, or —NR^{c}—;
R^{c} is hydrogen, C1-C10 alkyl, or substituted C1-C10 alkyl, or R^{c} and R¹ taken together with the nitrogen atom can form a heterocyclic ring or a substituted heterocyclic ring;
R¹ is hydrogen, C1-C10 alkyl, substituted C1-C10 alkyl, C1-C10 alkoxy, substituted C1-C10 alkoxy, C1-C10 alkyl C1-C10 alkoxy, substituted C1-C10 alkyl C1-C10 alkoxy, C5-C10 aryl, substituted C5-C10 aryl, C5-C9 heterocyclic, substituted C5-C9 heterocyclic, C3-C9 carbocyclic or substituted C3-C9 carbocyclic;
each R² independently is —OH, C1-C6 alkyl, substituted C1-C6 alkyl, C1-C6 alkoxy, substituted C1-C6 alkoxy, —C(O)—C1-C6 alkyl (alkanoyl), substituted —C(O)—C1-C6 alkyl, —C(O)—C6-C10 aryl (aroyl), substituted —C(O)—C6-C10 aryl, —C(O)OH (carboxyl), —C(O)O—C1-C6 alkyl (alkoxycarbonyl), substituted —C(O)O—C1-C6 alkyl, —NR^{a}R^{b}, —C(O)NR^{a}R^{b} (carbamoyl), substituted C(O)NR^{a}R^{b}, halo, nitro, or cyano;
the substituents on the alkyl, aryl or heterocyclic groups are hydroxy, C1-C6 alkyl, hydroxy C1-C6 alkylene, C1-C6 alkoxy, C3-C6 cycloalkyl, C1-C6 alkoxy C1-C6 alkylene, amino, cyano, halogen, or aryl;
each R^{a} and R^{b} is independently hydrogen, C1-C6 alkyl, C3-C8 cycloalkyl, C1-C6 alkoxy, halo C1-C6 alkyl, C3-C8 cycloalkyl C1-C6 alkyl, C1-C6 alkanoyl, hydroxy C1-C6 alkyl, aryl, aryl C1-C6 alkyl, Het, Het C1-C6 alkyl, or C1-C6 alkoxycarbonyl;
each X² independently is a bond or a linking group;
each R³ independently is a polyethylene glycol (PEG) moiety;
each R⁴ independently is H, —C1-C6 alkyl, —C1-C6 alkoxy, —NR^{a}R^{b}, —OH, —CN, —COOH, —COOR¹, —C1-C6 alkyl-NR^{a}R^{b}, —C1-C6 alkyl-OH, —C1-C6 alkyl-CN, —C1-C6 alkyl-COOH, —C1-C6 alkyl-COOR¹, —R—CS—NR′R—, -optionally substituted 5-6 membered ring, or —C₁-C₆ alkyl-optionally substituted 5-6 membered ring;
m is 1, 2, 3, 4, 5, 6, 7, 8, 9 or 10;
n is 0, 1, 2, 3 or 4;
p is 1 to 100;
q is 1, 2, 3, 4 or 5
r is 1 to 1,000;
s is 1 to 1,000; and
the sum of n and q equals 5
or a pharmaceutically acceptable salt, tautomer, or hydrate thereof, wherein the condition is organ fibrosis.

2. The method of claim 1, wherein the compound has a structure according to Formula II: 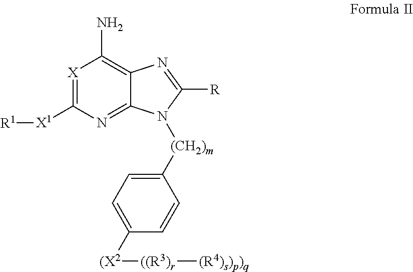
or a pharmaceutically acceptable salt thereof, or a hydrate thereof, where X, X¹, X², R, R¹, R², R³, R⁴, m, n, p, q, r and s embodiments are described above for Formula I.

3. (canceled)

4. The method of claim 1, wherein X is N, X¹ is O, and R is OH.

5. The method of claim 1, wherein m is 1, n is 0, p is 1, and q is 1, and X² is a linking group.

6. The method of claim 1, wherein R³ is PEG.

7. The method of claim 1, wherein r is 6 to 10.

8. The method of claim 1, wherein s is 3 and each R⁴ is selected from the group consisting of C1 to C6 alkyl, an optionally substituted 5 or 6-membered ring, and C1 to C6 alkyl COOH.

9. The method of claim 1, wherein X² is C(O)NH.

10. The method of claim 1, wherein the compound is a compound of Table 2.

11. The method of claim 1, wherein the compound is Compound 2.

12. The method of claim 1, wherein the compound is Compound 6.

13. The method of claim 1, wherein the condition is selected from the group consisting of lung fibrosis, endomyocardial fibrosis, renal fibrosis, keloid, mediastinal fibrosis, myelofibrosis, nephrogenic systemic fibrosis, progressive massive fibrosis, pulmonary and idiopathic pulmonary fibrosis and retroperitoneal fibrosis.

14. The method of claim 1, wherein the condition is lung fibrosis.

15. The method of claim 1, wherein the condition is renal fibrosis.

16-19. (canceled)

20. The method of claim 1, wherein the subject is human.

21. The method of claim 1, further comprising administering an anti-inflammatory compound.

22. A method for preventing, inhibiting or treating organ fibrosis in a subject, which comprises administering a compound having the following structure: 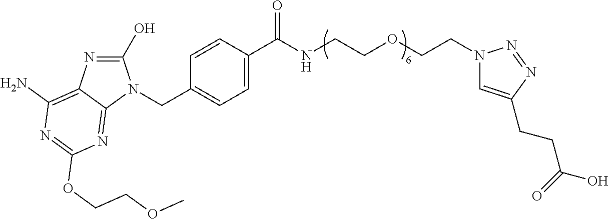
or a pharmaceutically acceptable salt thereof or hydrate thereof.

23. A method for treating a condition in a subject, comprising administering to the subject a compound having a structure according to Formula I: 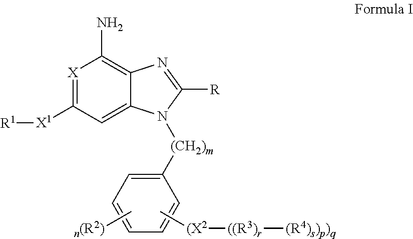
or a pharmaceutically acceptable salt thereof, including a hydrate thereof, wherein:
X is N or CR²;
R is —OR¹, —SR¹, or —NR^{a}R^{b},
X¹ is a bond or is —O—, —S—, or —NR^{c}—;
R^{c} is hydrogen, C1-C10 alkyl, or substituted C1-C10 alkyl, or R^{c} and R¹ taken together with the nitrogen atom can form a heterocyclic ring or a substituted heterocyclic ring;
R′ is hydrogen, C1-C10 alkyl, substituted C1-C10 alkyl, C1-C10 alkoxy, substituted C1-C10 alkoxy, C1-C10 alkyl C1-C10 alkoxy, substituted C1-C10 alkyl C1-C10 alkoxy, C5-C10 aryl, substituted C5-C10 aryl, C5-C9 heterocyclic, substituted C5-C9 heterocyclic, C3-C9 carbocyclic or substituted C3-C9 carbocyclic;
each R² independently is —OH, C1-C6 alkyl, substituted C1-C6 alkyl, C1-C6 alkoxy, substituted C1-C6 alkoxy, —C(O)—C1-C6 alkyl (alkanoyl), substituted —C(O)—C1-C6 alkyl, —C(O)—C6-C10 aryl (aroyl), substituted —C(O)—C6-C10 aryl, —C(O)OH (carboxyl), —C(O)O—C1-C6 alkyl (alkoxycarbonyl), substituted —C(O)O—C1-C6 alkyl, —NR^{a}R^{b}, —C(O)NR^{a}R^{b} (carbamoyl), substituted C(O)NR^{a}R^{b}, halo, nitro, or cyano;
the substituents on the alkyl, aryl or heterocyclic groups are hydroxy, C1-C6 alkyl, hydroxy C1-C6 alkylene, C1-C6 alkoxy, C3-C6 cycloalkyl, C1-C6 alkoxy C1-C6 alkylene, amino, cyano, halogen, or aryl;
each R^{a} and R^{b} is independently hydrogen, C1-C6 alkyl, C3-C8 cycloalkyl, C1-C6 alkoxy, halo C1-C6 alkyl, C3-C8 cycloalkyl C1-C6 alkyl, C1-C6 alkanoyl, hydroxy C1-C6 alkyl, aryl, aryl C1-C6 alkyl, Het, Het C1-C6 alkyl, or C1-C6 alkoxycarbonyl;
each X² independently is a bond or a linking group;
each R³ independently is a polyethylene glycol (PEG) moiety;
each R⁴ independently is H, —C1-C6 alkyl, —C1-C6 alkoxy, —NR^{a}R^{b}, —OH, —CN, —COOH, —COOR¹, —C1-C6 alkyl-NR^{a}R^{b}, —C1-C6 alkyl-OH, —C1-C6 alkyl-CN, —C1-C6 alkyl-COOH, —C1-C6 alkyl-COOR¹, —R—CS—NR′R—, -optionally substituted 5-6 membered ring, or —C₁-C₆ alkyl-optionally substituted 5-6 membered ring;
m is 1, 2, 3, 4, 5, 6, 7, 8, 9 or 10;
n is 0, 1, 2, 3 or 4;
p is 1 to 100;
q is 1, 2, 3, 4 or 5
r is 1 to 1,000;
s is 1 to 1,000; and
the sum of n and q equals 5
or a pharmaceutically acceptable salt, tautomer, or hydrate thereof, wherein the condition is kidney failure or kidney disease.

24. The method of claim 22, wherein the condition is renal fibrosis.
